(19) United States
(12) Patent Application Publication
(10) Pub. No.: US 2018/0354931 A1
(43) Pub. Date: Dec. 13, 2018

(54) SPIROCYCLIC DERIVATIVE, AND POLYMER, MIXTURE, FORMULATION AND ORGANIC ELECTRONIC DEVICE COMPRISING THE SAME

(71) Applicant: GUANGZHOU CHINARAY OPTOELECTRONIC MATERIALS LTD., Guangzhou, Guangdong (CN)

(72) Inventors: Ruifeng HE, Guangzhou, Guangdong (CN); Peng SHU, Guangzhou, Guangdong (CN); Jun WANG, Guangzhou, Guangdong (CN); Junyou PAN, Guangzhou, Guangdong (CN)

(73) Assignee: GUANGZHOU CHINARAY OPTOELECTRONIC MATERIALS LTD., Guangzhou, Guangdong (CN)

(21) Appl. No.: 15/781,377

(22) PCT Filed: Sep. 12, 2016
(86) PCT No.: PCT/CN2016/098739

§ 371 Date: Jun. 4, 2018

(30) Foreign Application Priority Data
Dec. 4, 2015 (CN) 201510889328.8

(51) Int. Cl. C07D 401/14; C07D 401/10; C07D 401/04; C07D 407/10; C07D 409/10; C07D 209/82; C07D 307/91; H01L 51/00; H01L 51/50

(52) U.S. Cl. CPC C07D 401/14; H01L 51/5056; C07D 401/04; C07D 407/10; C07D 409/10; C07D 209/82; C07D 307/91; H01L 51/0058; H01L 51/0072; H01L 51/0073; H01L 51/0074; H01L 51/0067; H01L 51/006; H01L 51/5016; C07D 401/10

(57) ABSTRACT

Provided are a spirocyclic derivative, and a high polymer, a mixture, a composition and an organic electronic device containing same, wherein in the spirocyclic derivative, two spirocyclic units are directly or indirectly connected by a sp3 hybridized carbon atom, thus effectively adjusting the energy level of the compound, and being beneficial for improving the photoelectric performance of the compound and the stability of the device. An effective solution is provided for effectively reducing the manufacturing cost and improving the efficiency and lifetime of a light-emitting device.

### TECHNICAL FIELD

The present disclosure relates to the field of novel organic opto-electronic material, particularly to a spirocyclic derivative, and a polymer, a mixture, a formulation and an organic electronic device comprising the same.

### BACKGROUND

With the characteristics of structural diversity, relatively low manufacturing cost, superior opto-electronic property, etc., organic semiconductor materials show great potential for a use in optoelectronic devices such as organic light-emitting diode (OLED), such as flat panel displays and lighting.

In order to improve the luminescence properties of the organic light-emitting diodes and promote the large-scale industrialization process of the organic light-emitting diodes, a variety of new structural material systems with organic opto-electronic properties have been widely developed. However, the properties of OLED, especially the lifetime of OLED has yet to be improved. For example, in phosphorescent OLED, the stability of host material determines the lifetime of the devices. For another example, a new generation OLED material, i.e. thermally activated delayed fluorescent emitter (TADF), has quite a high efficiency, but very low lifetime, mainly due to no suitable host material. For still another example, in the printed OLED, there is an urgent need for material, especially host material, having good performance, solubility, film-forming property, and thermal stability. Hence, it is urgent need for development of a novel high-performance host material.

Among various materials, a spirocyclic derivative, such as spirofluorene and the like have been widely used in optoelectronic devices due to their excellent opto-electronic response and carrier transmission performance. However, the spirocyclic derivative reported currently still has certain limitation in the aspect of opto-electronic performance. New spirocyclic derivative structure should be developed for further exploring the opto-electronic property of such material.

### SUMMARY OF THE INVENTION

In view of this, it is necessary to provide a spirocyclic derivative having better opto-electronic performance, a polymer, a mixture, a formulation and an organic electronic device comprising the same.

A spirocyclic derivative comprises the following general formula (I)

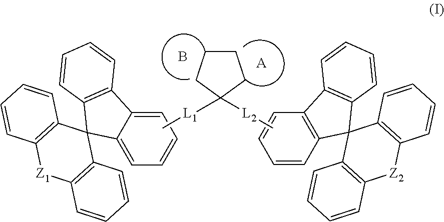

wherein, L₁ or L₂ is a single bond, an aromatic group containing 6 to 40 carbon atoms, or a heteroaromatic group containing 3 to 40 carbon atoms.

A or B is an aromatic group containing 6 to 20 carbon atoms or a heteroaromatic group containing 3 to 20 carbon atoms.

Z₁ or Z₂ is selected from a single bond, N(R), B(R), C(R)₂, Si(R)₂, O, S, C═N(R), C═(R)₂, P(R), P(═O)R, S═O, or SO₂, or is absent.

The hydrogen atoms on L₁, L₂, A, B and the spirocyclic derivative can be substituted by R;

R is selected from an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms, and one or more positions of R may be substituted by H, D, F, CN, alkyl, aralkyl, alkenyl, alkynyl, nitrile group, amino, nitro, acyl, alkoxy, carbonyl, sulfonyl, cycloalkyl or hydroxy.

A polymer comprises a repeating unit including the spirocyclic derivative described above.

A mixture comprises the above spirocyclic derivative or the above polymer;

The mixture further includes an organic functional material.

A formulation comprises the above spirocyclic derivative, the above polymer or the above mixture;

The mixture further comprises an organic solvent.

An organic electronic device comprises the above spirocyclic derivative or the above polymer.

Such spirocyclic derivative, when is applied in OLED, especially used as a material for light-emitting layer, can provide high light-emission stability and lifetime of device. Such spirocyclic derivative has relatively suitable ground state and excited state level, and excellent carrier transport property, high fluorescence characteristics and structural stability, and better opto-electronic performance compared with the traditional materials.

### DETAILED DESCRIPTION OF THE EMBODIMENTS

In order to make the above objects, features, and advantages of the present disclosure to be understood more clearly, the specific embodiments of the present disclosure will be described in detail below with reference to the accompanying drawings and specific examples. In the following description, numerous specific details are set forth in order to provide a thorough understanding of the present disclosure. Rather the present disclosure can be implemented in many other different ways from those described herein, and similar improvements may be made by those skilled in the art without departing from the spirit of the present disclosure. Therefore, the present disclosure is not limited by the specific embodiments disclosed below.

In the present disclosure, the formulation and the printing ink, or the ink, have the same meaning and they are interchangeable.

In the present disclosure, the host material and the matrix material have the same meaning and they are interchangeable.

In the present disclosure, the metal organic clathrate, the metal organic complexes, and organometallic complexes have the same meaning and are interchangeable.

A spirocyclic derivative comprises the following general formula (I)

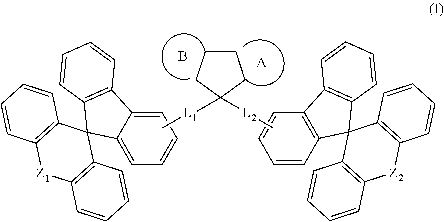

wherein, L₁ or L₂ is a single bond, an aromatic group containing 6 to 40 carbon atoms, or a heteroaromatic group containing 3 to 40 carbon atoms.

A or B is an aromatic group containing 6 to 20 carbon atoms or a heteroaromatic group containing 3 to 20 carbon atoms.

Z₁ or Z₂ is selected from a single bond, N(R), B(R), C(R)₂, Si(R)₂, O, S, C═N(R), C═C(R)₂, P(R), P(═O)R, S═O, or SO₂, or absent.

The hydrogen atoms on L₁, L₂, A, B and the spirocyclic derivative can be substituted by R;

R is selected from an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms, and one or more positions of R may be substituted by H, D, F, CN, alkyl, aralkyl, alkenyl, alkynyl, nitrile group, amino, nitro, acyl, alkoxy, carbonyl, sulfonyl, cycloalkyl, or hydroxy.

In one embodiments, L₁ or L₂ is an aromatic group containing 6 to 30 carbon atoms, or a heteroaromatic group containing 3 to 30 carbon atoms.

In another embodiment, L₁ or L₂ is an aromatic group containing 6 to 25 carbon atoms, or a heteroaromatic group containing 3 to 25 carbon atoms.

In another preferred embodiment, L₁ or L₂ is an aromatic group containing 6 to 20 carbon atoms, or a heteroaromatic group containing 3 to 20 carbon atoms.

In one embodiment, A or B is an aromatic group containing 6 to 18 carbon atoms or a heteroaromatic group containing 3 to 18 carbon atoms.

In another embodiment, A or B is an aromatic group containing 6 to 15 carbon atoms or a heteroaromatic group containing 3 to 15 carbon atoms.

In one embodiment, Z₁ or Z₂ is selected from a single bond, N(R), C(R)₂, O, or S.

Heteroaryl groups refer to hydrocarbyl groups (containing heteroatoms) that contain at least one heteroaryl ring, including monocyclic groups and polycyclic ring systems. These polycyclic rings may have two or more rings in which two carbon atoms are shared by two adjacent rings, i.e., a fused ring. At least one of these polycyclic rings is heteroaryl.

Specifically, examples of aryl groups include benzene, naphthalene, anthracene, phenanthrene, perylene, tetracene, pyrene, benzopyrene, triphenylene, acenaphthene, fluorene, and derivatives thereof.

Specifically, examples of heteroaryl group are: furan, benzofuran, thiophene, benzothiophene, pyrrol, pyrazole, triazole, imidazole, oxazole, oxadiazole, thiazole, tetrazole, indole, carbazole, pyrroloimidazole, pyrrolopyrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, perimidine, quinazoline, quinazolinone, and derivatives thereof.

In an embodiment, L₁ or L₂ is benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, spirosilabifluorene and the like. In a preferred embodiment, L₁ or L₂ is a single bond, or a group, such as benzene, pyridine, pyrimidine, triazine, carbazole, and the like.

In another preferred embodiment, L₁ or L₂ comprises one of the following groups:

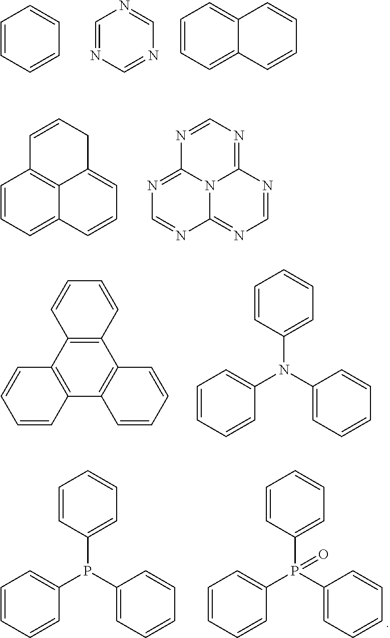

In a more preferred embodiment, A or B comprises one of the following groups:

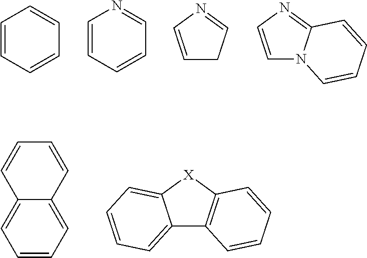

wherein, X is N(R₁), B(R₁), C(R₁)₂, Si(R₁)₂, O, S, C═N(R₁), C═C(R₁)₂, P(R₁), P(═O)R₁, S═O or SO₂, and in a preferred embodiment, X is N(R₁), C(R₁)₂, O or S.

R₁ is selected from H, D, F, CN, aralkyl, alkenyl, alkynyl, nitrile group, amino, nitro, acyl, alkoxy, carbonyl sulfonyl, hydroxyl, an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms

In one embodiment, R₁ is selected from the group consisting of methyl, benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, spirosilabifluorene and the like.

In another embodiment, R₁ is selected from the group consisting of benzene, pyridine, pyrimidine, triazine, carbazole, and the like.

Two spirocyclic unit of the spirocyclic derivatives of the present disclosure are linked to a sp³-hybridized carbon atom by L₁ and L₂.

In a more preferred embodiment the spirocyclic derivative of the present disclosure is one selected from compounds having the following structural formula:

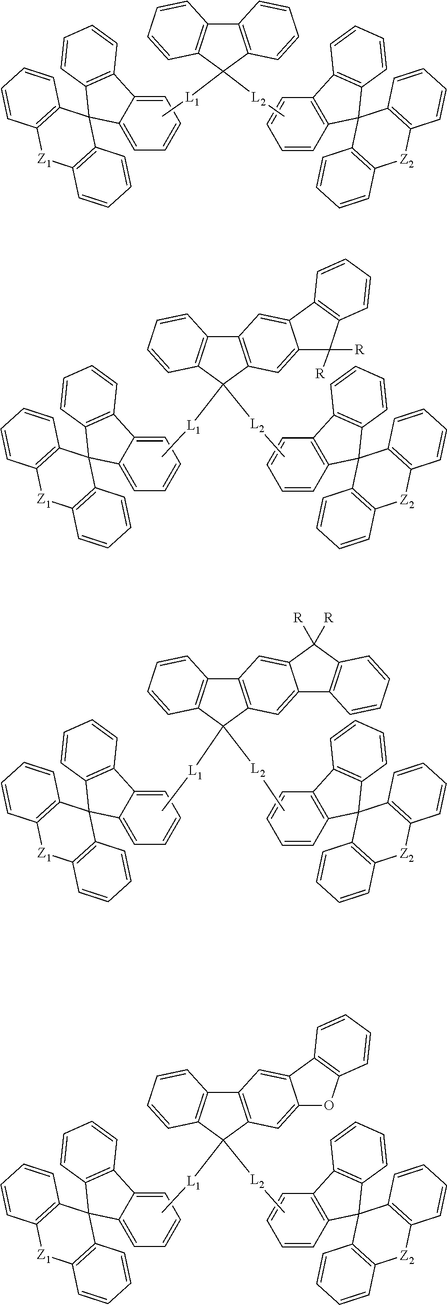

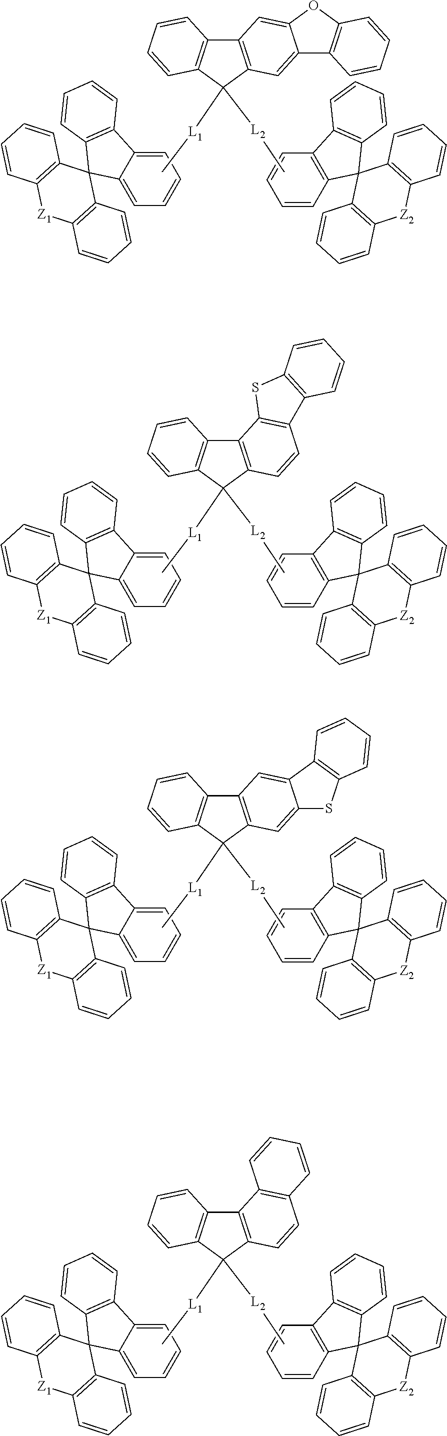

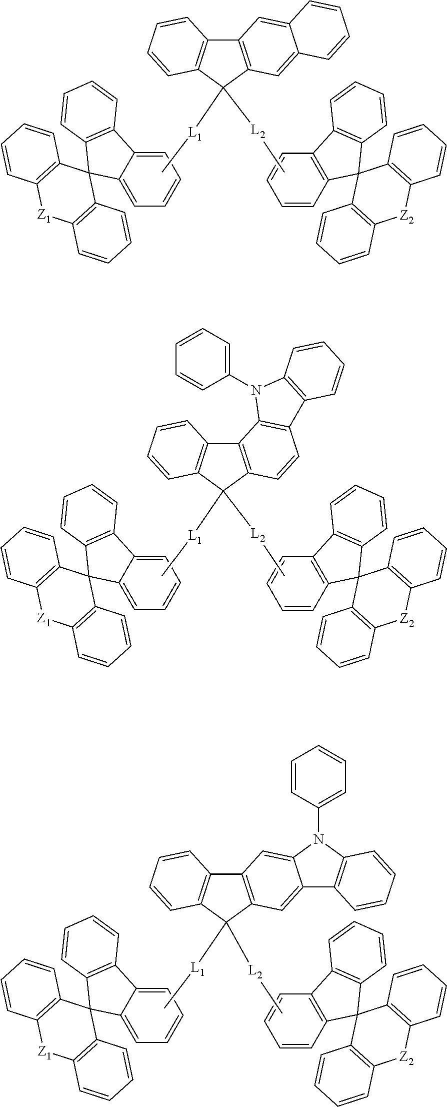

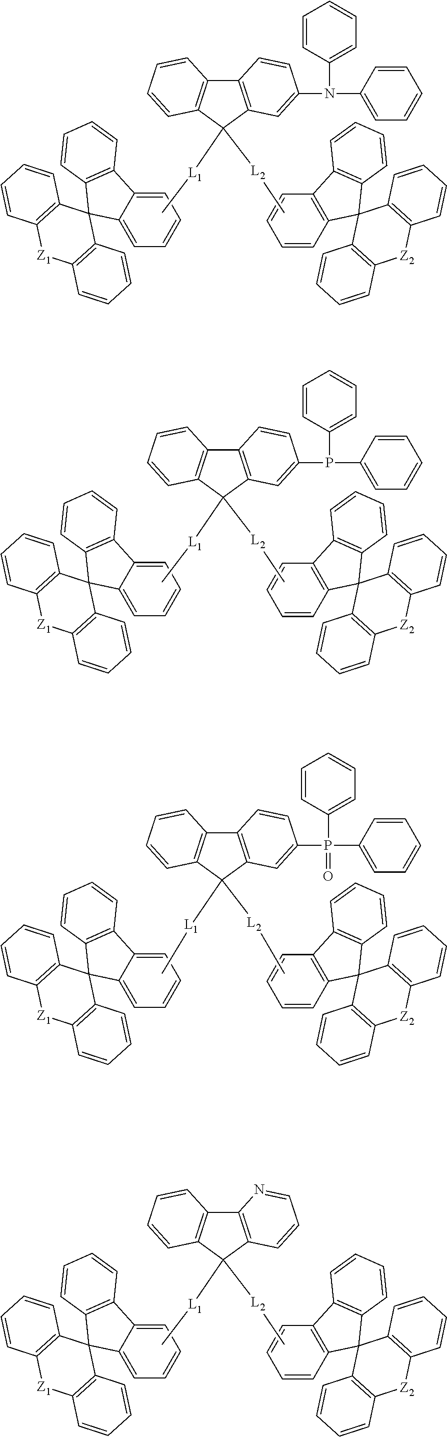

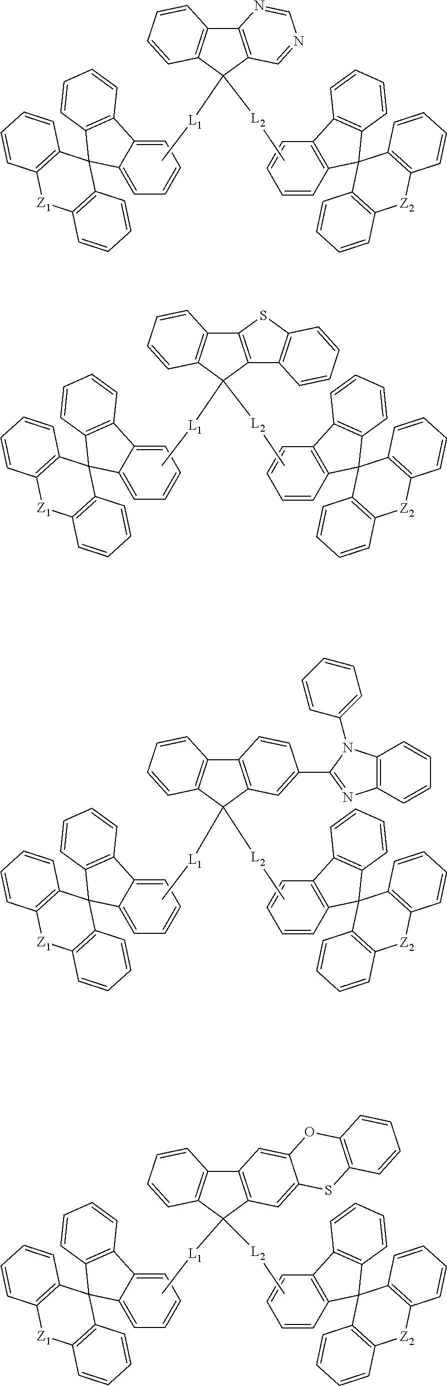

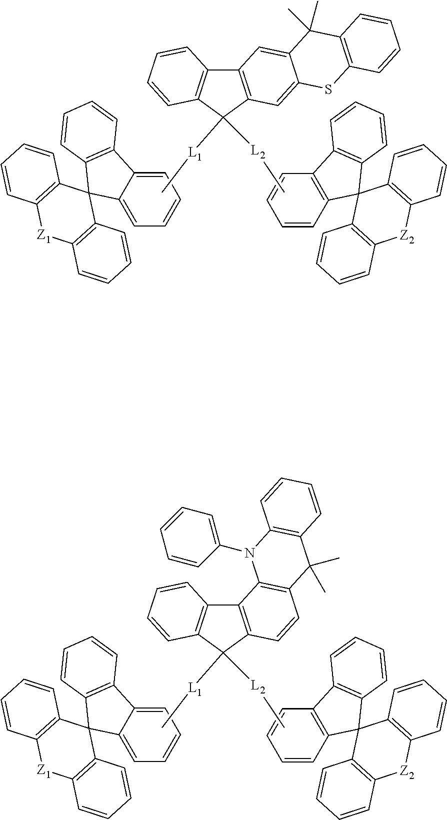

wherein, Z₁, Z₂, L₁, L₂ and R are as defined above.

In a more preferred embodiment, the spirocyclic derivative of the present disclosure is one selected from compounds having the following structural formula:

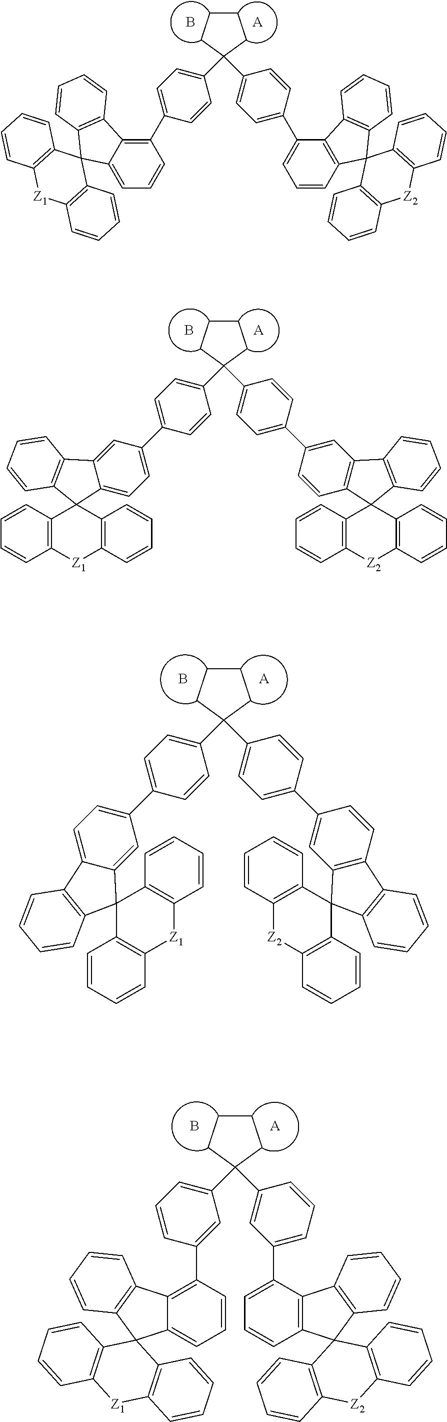

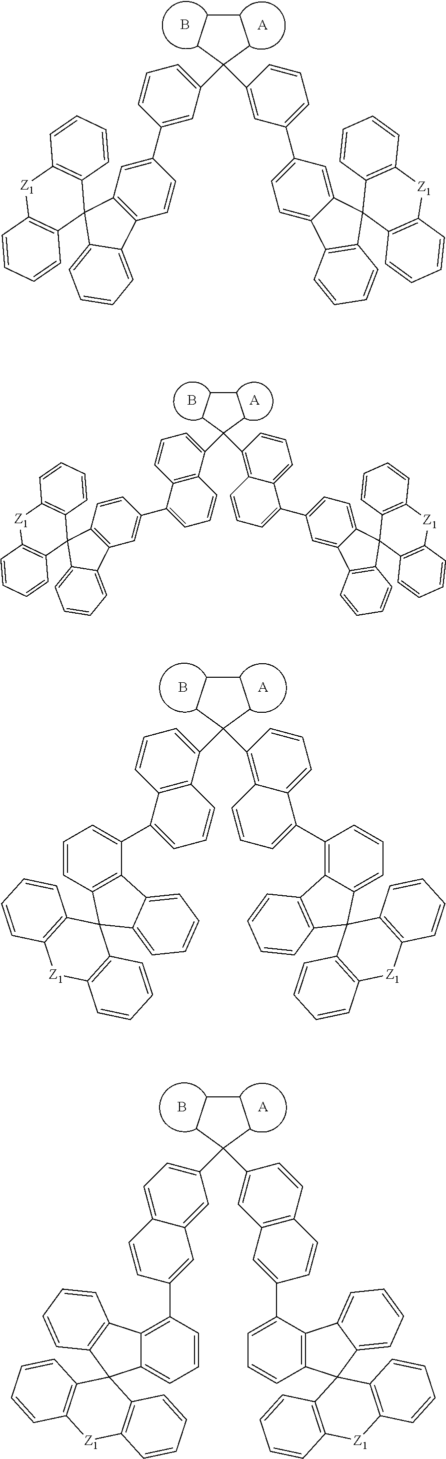

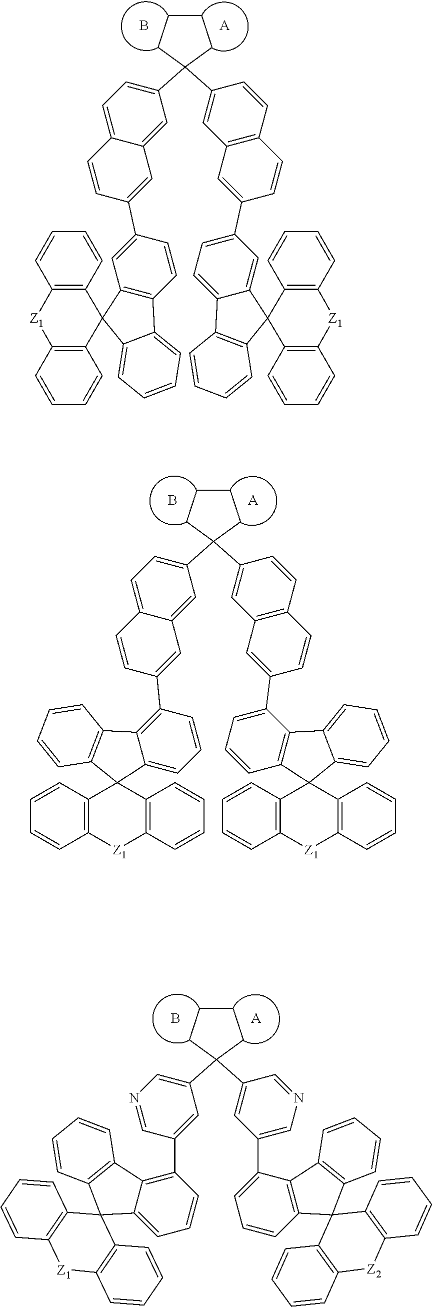

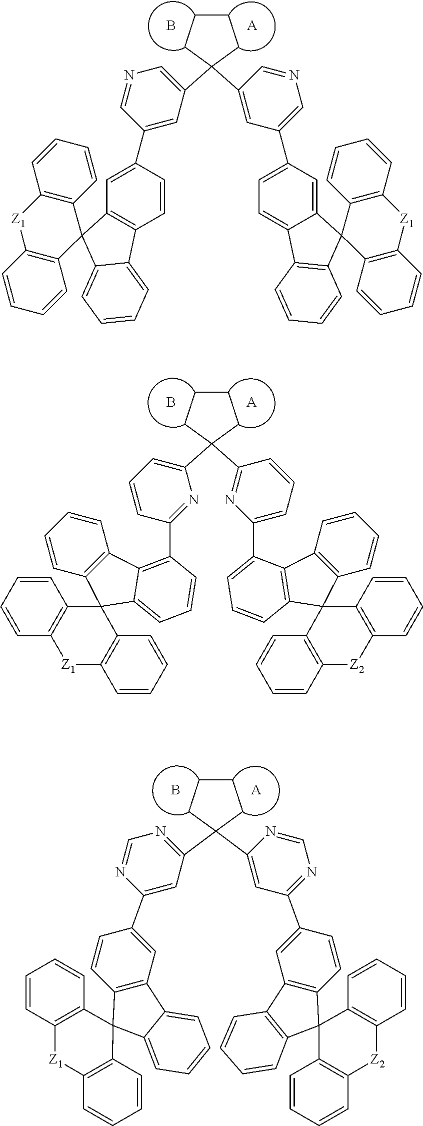

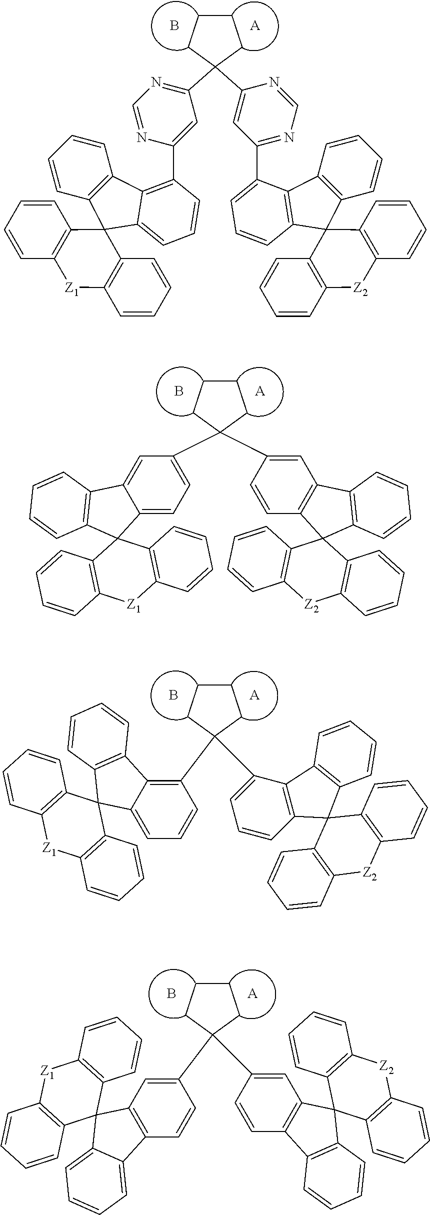

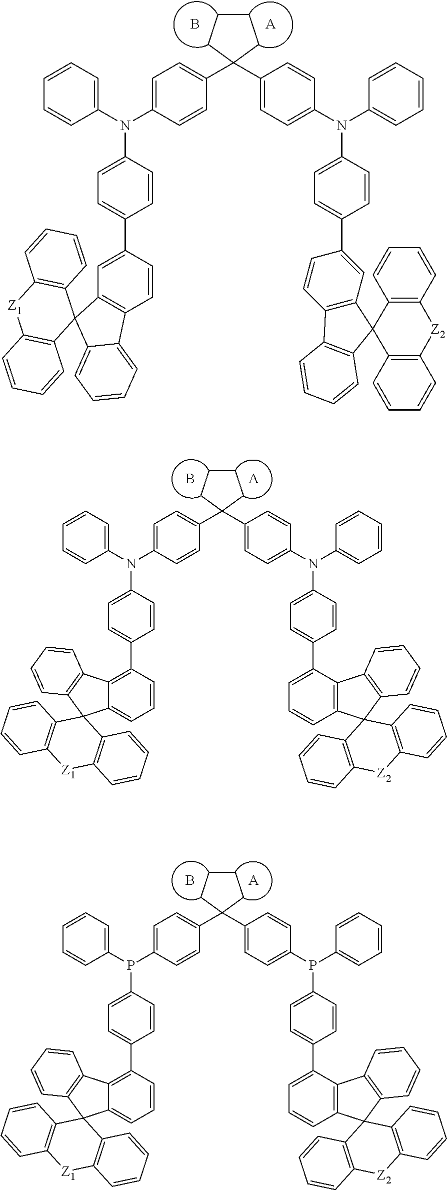

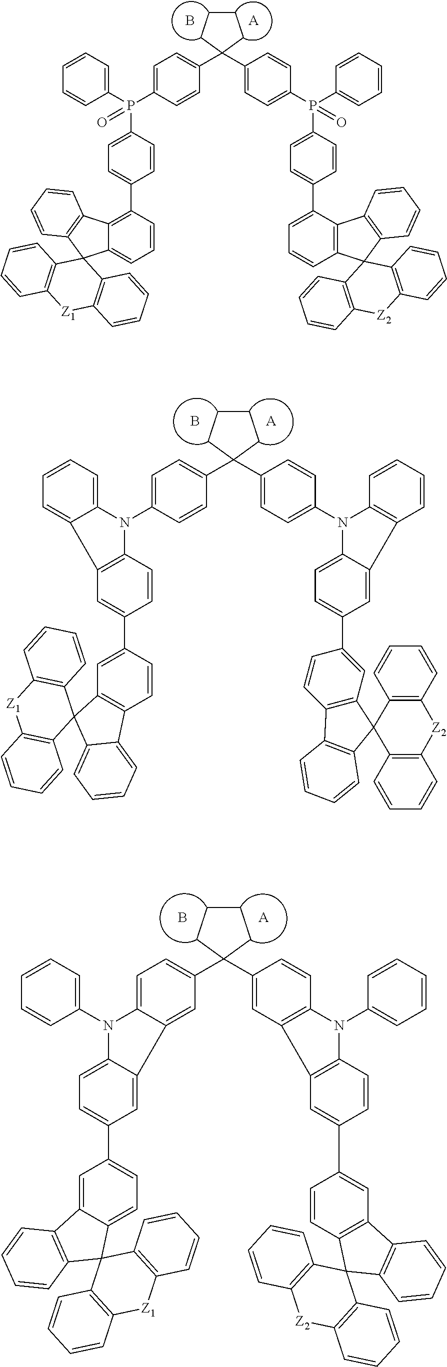

wherein, Z₁, Z₂, A and B are as defined above.

The spirocyclic derivative of the present disclosure can be used in electronic devices as a functional material. Organic functional materials may be classified as a hole-injection material (HIM), a hole-transport material (HTM), an electron-transport material (ETM), an electron-injection material (EIM), an electron-blocking material (EBM), a hole-blocking material (HBM), an emitter, or a host material.

In a preferred embodiment, the spirocyclic derivative in the present disclosure can be used as a host material, an electron-transport material or a hole-transport material.

In a more preferred embodiment, the spirocyclic derivative in the present disclosure can be used as a phosphorescent host material.

The spirocyclic derivative, as a phosphorescent host material must have a proper triplet energy level, i.e., T₁. In certain embodiments, the spirocyclic derivative in the present disclosure has a T₁ greater than or equal to 2.2 eV, in a preferred embodiment, the spirocyclic derivative in the present disclosure has a T₁ greater than or equal to 2.4 eV, in a more preferred embodiment, the spirocyclic derivative in the present disclosure has a T₁ greater than or equal to 2.6 eV in a still more preferred embodiment, the spirocyclic derivative in the present disclosure has a T₁ greater than or equal to 2.65 eV, and in a most preferred embodiment, the spirocyclic derivative in the present disclosure has a T₁ greater than or equal to 2.7 eV.

A phosphorescent host material is expected to have good thermal stability.

In general, the spirocyclic derivative of the present disclosure has a glass transition temperature T_{g} greater than or equal to 100° C. In a preferred embodiment T_{g}≥120° C. In a more preferred embodiment, T_{g}≥140° C. In a still more preferred embodiment T_{g}≥160° C. In a most preferred embodiment, T_{g}≥180° C.

In the synthesis of the spirocyclic derivative of the present disclosure, generally, a compound containing a hydroxyl group is made by a lower group of SP³ carbon atom, and then the hydroxyl group is oxidized into a carbonyl group; a lithium salt or a Grignard reagent is made from the upper group of the Sp³ carbon atom to attack the carbonyl group of the lower group; then a ring-closing reaction is performed so as to yield the spirocyclic derivative of the present disclosure.

Specific examples of the spirocyclic derivatives of the present disclosure are given below, but not limited to:

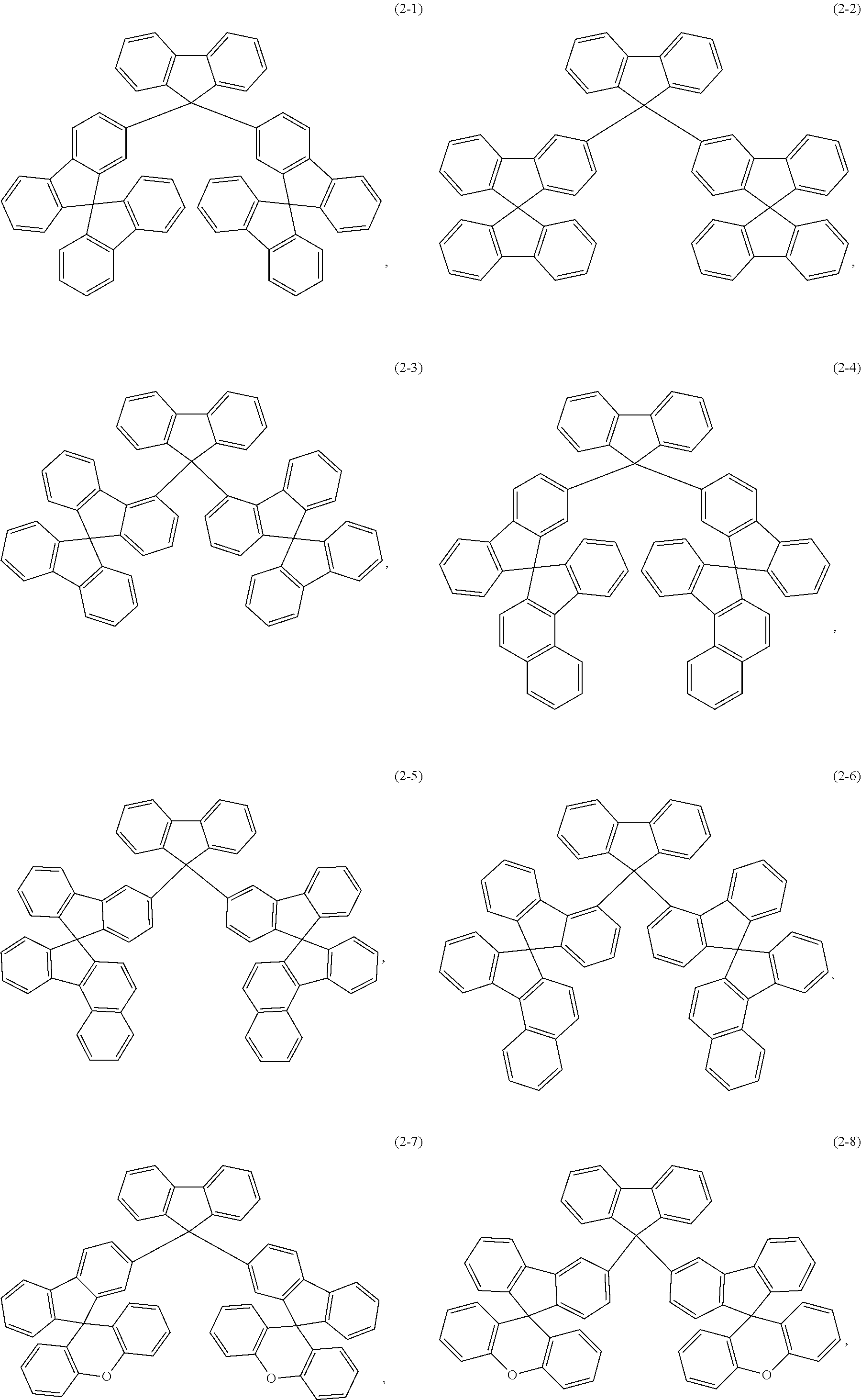

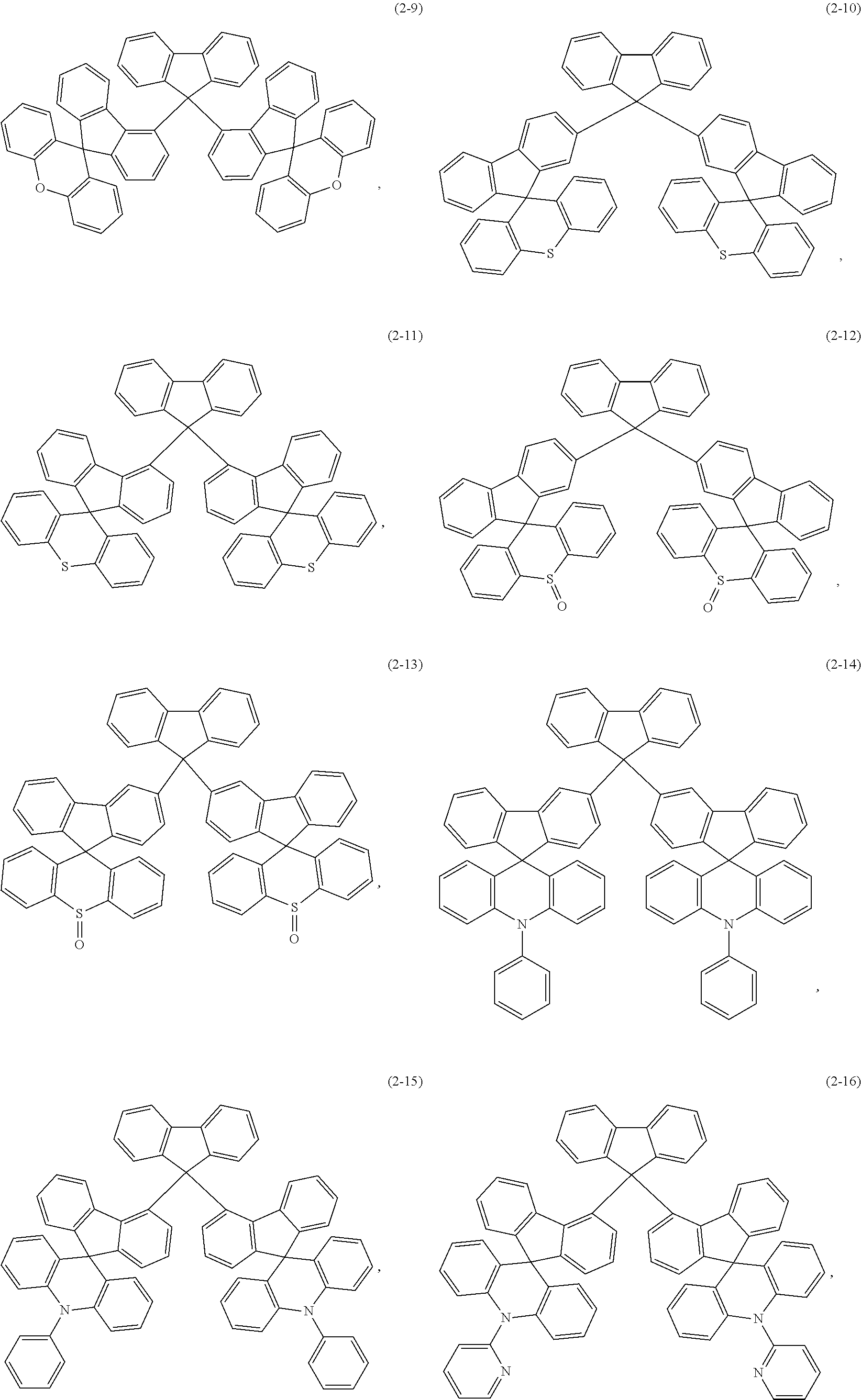

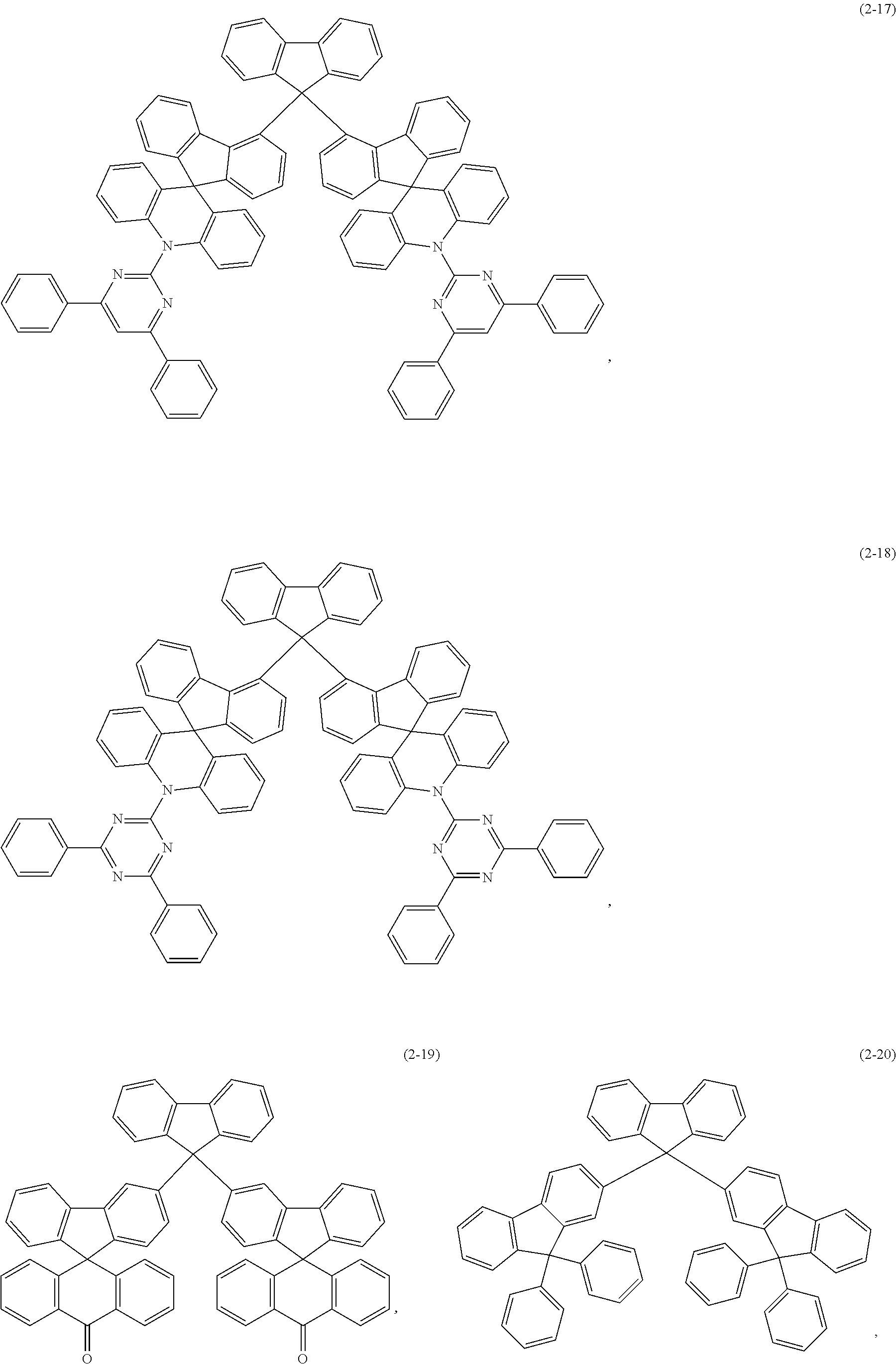

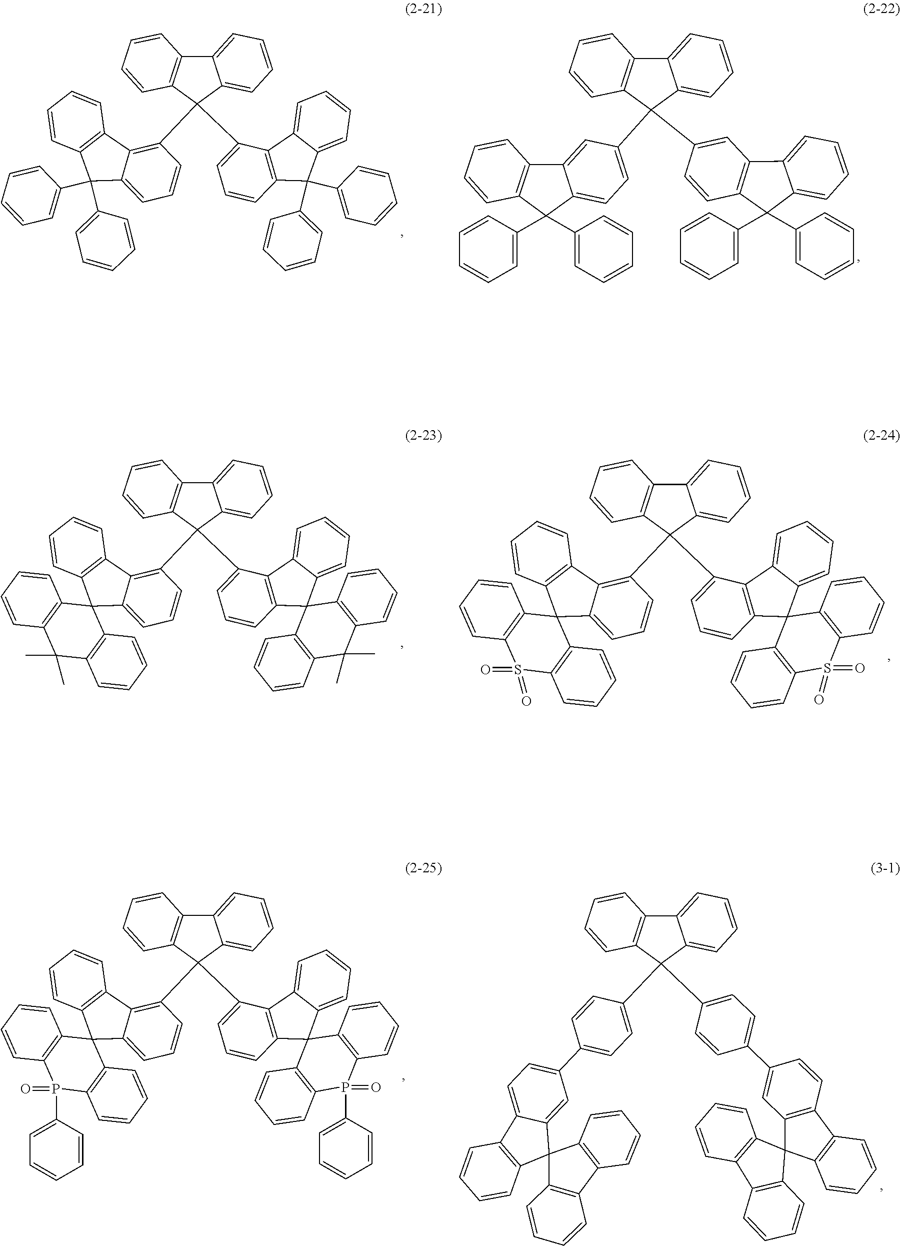

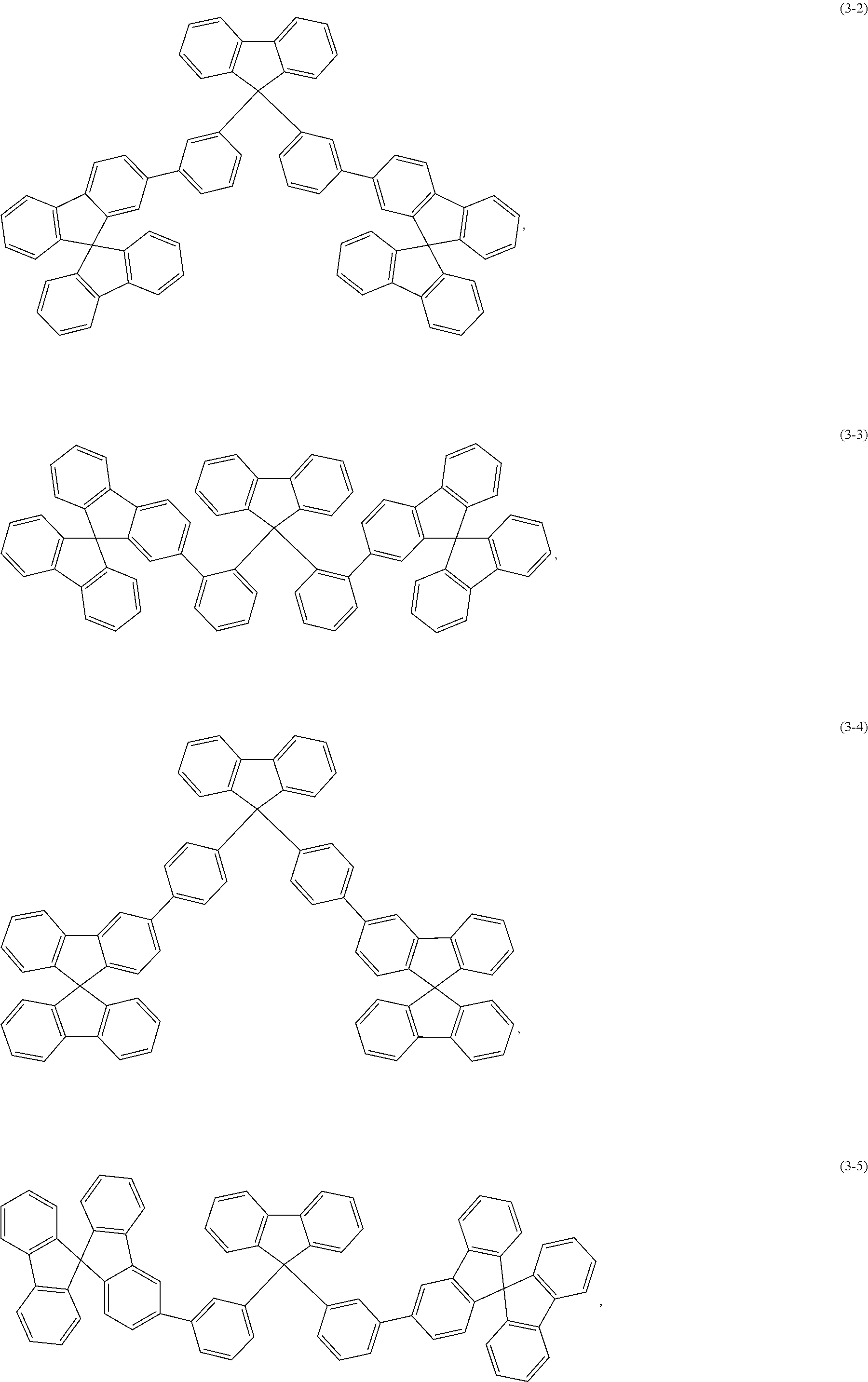

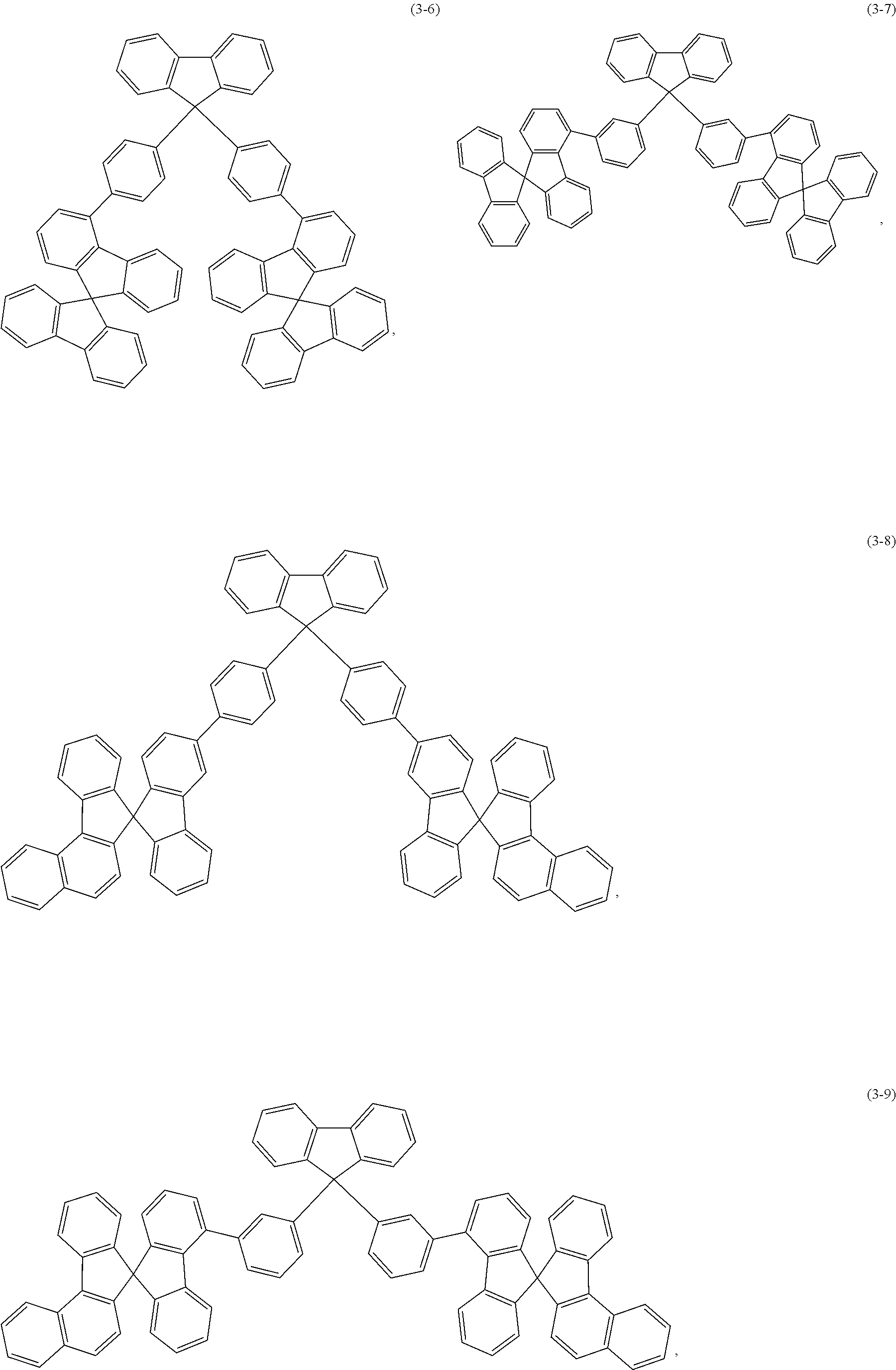

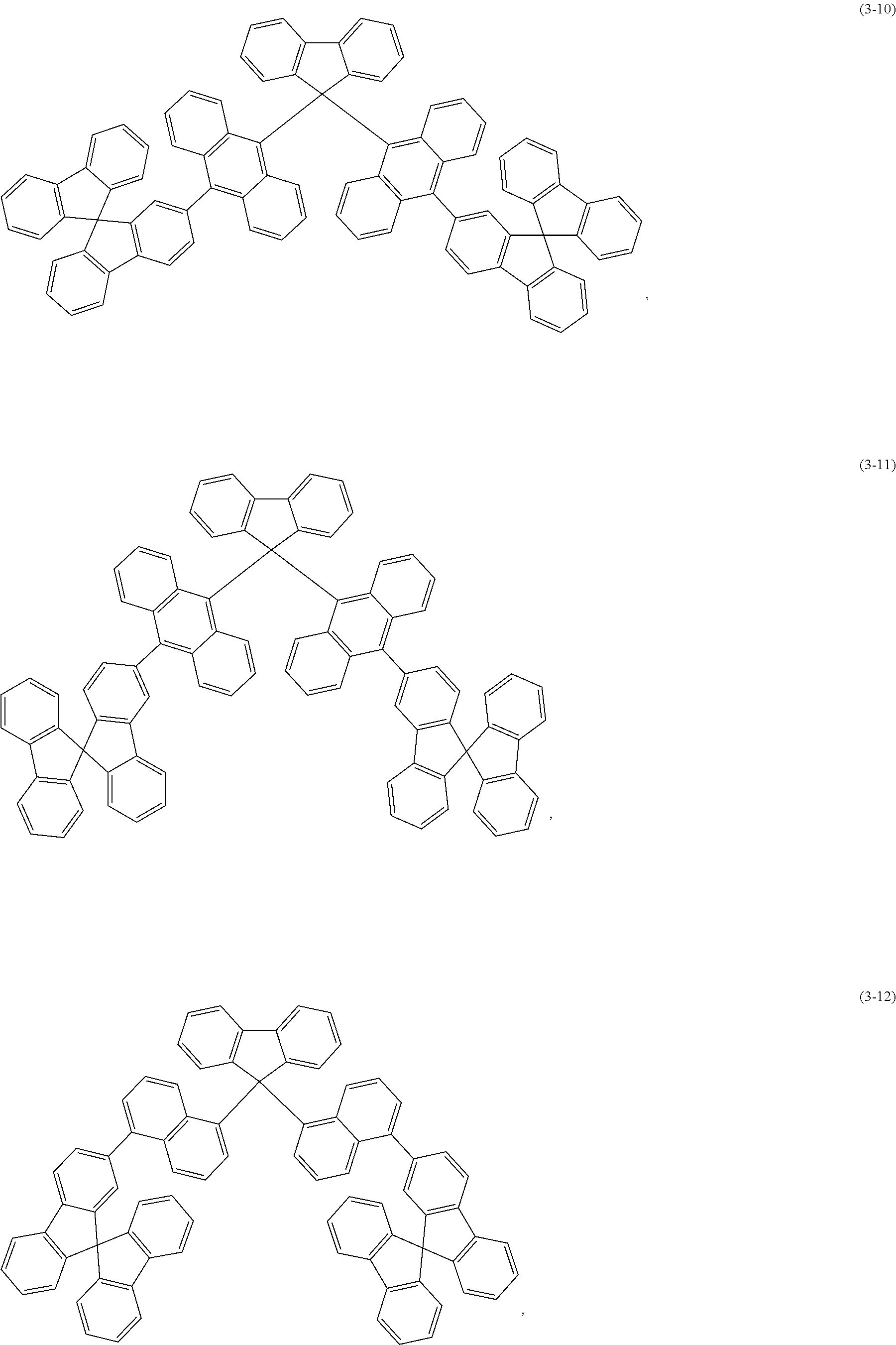

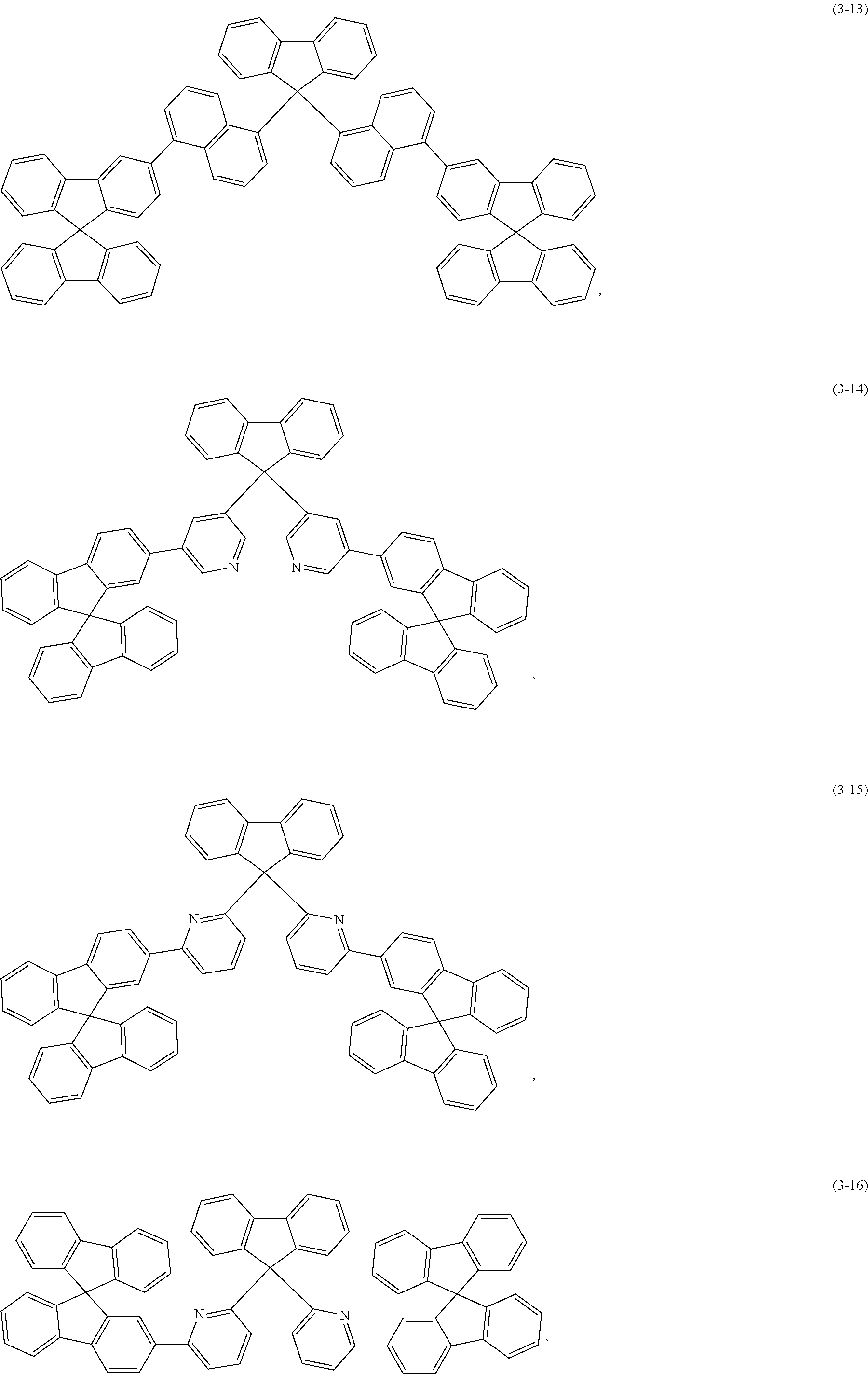

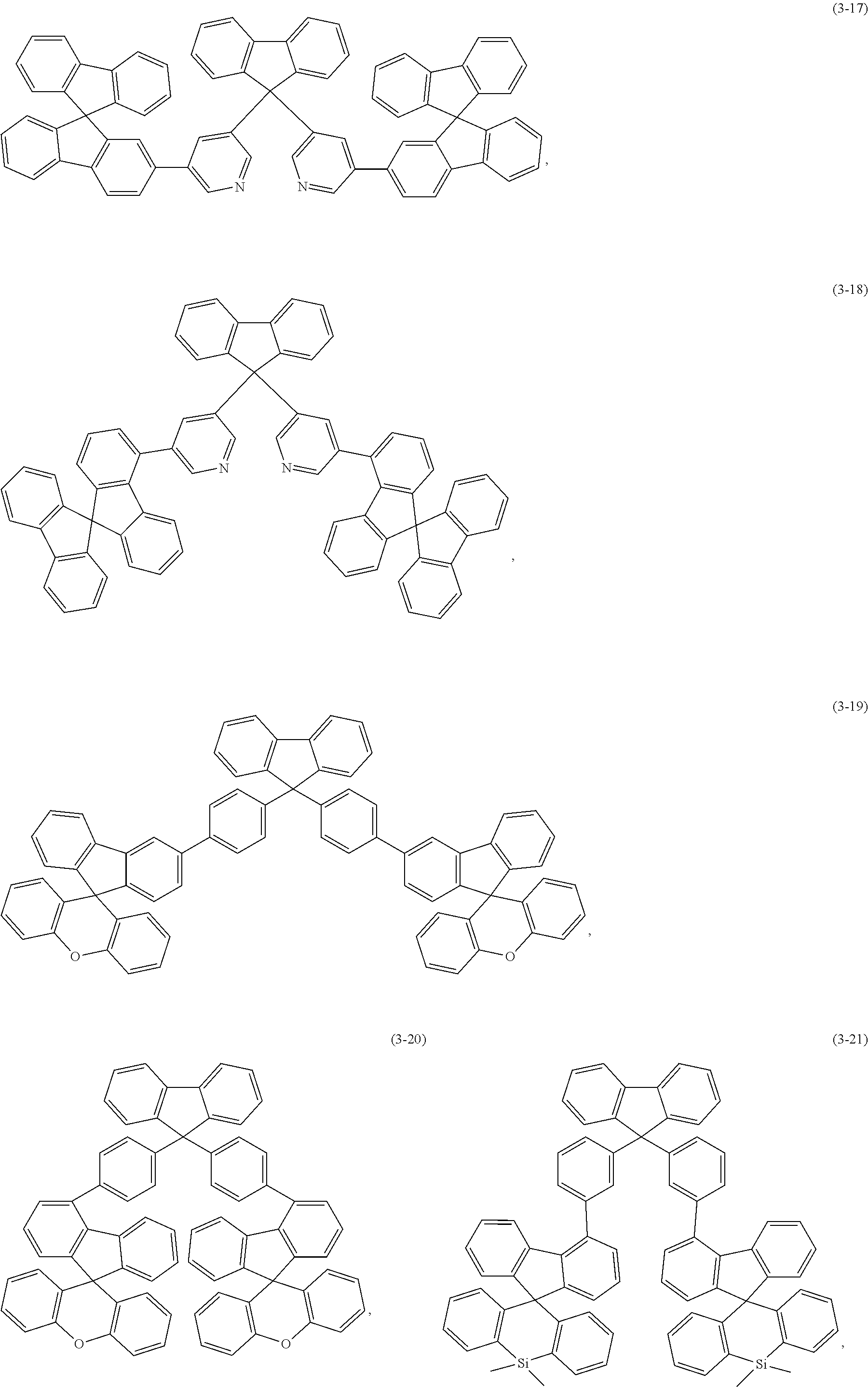

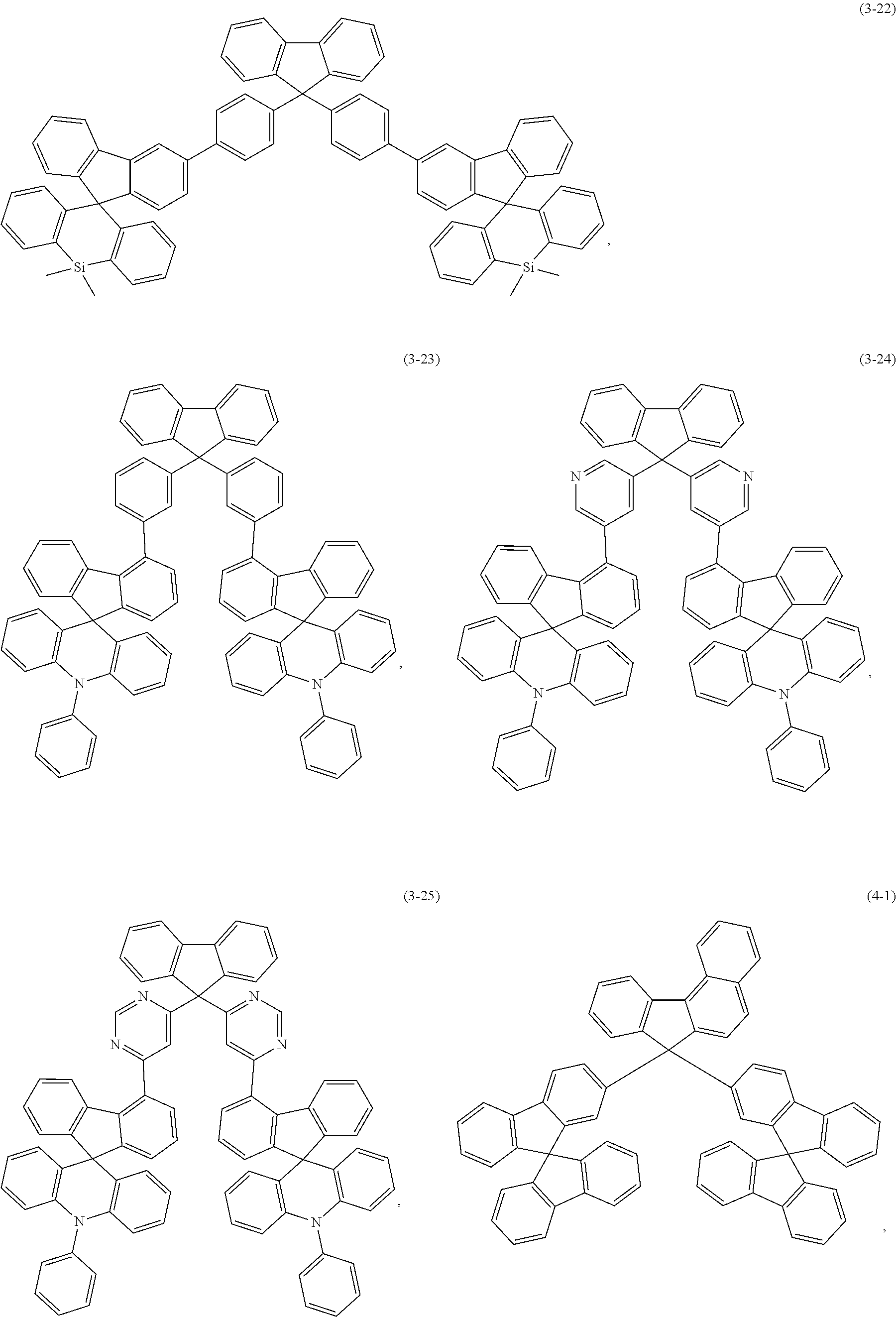

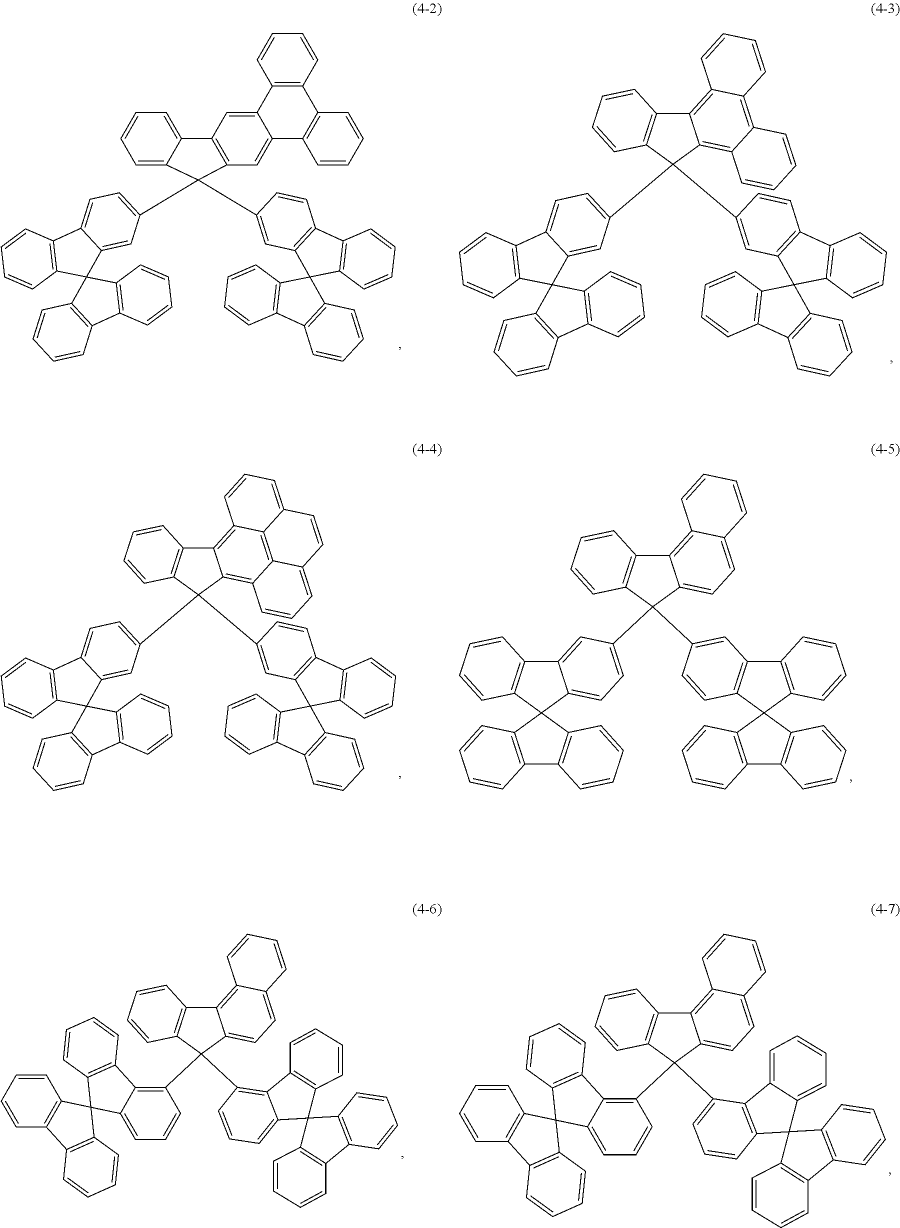

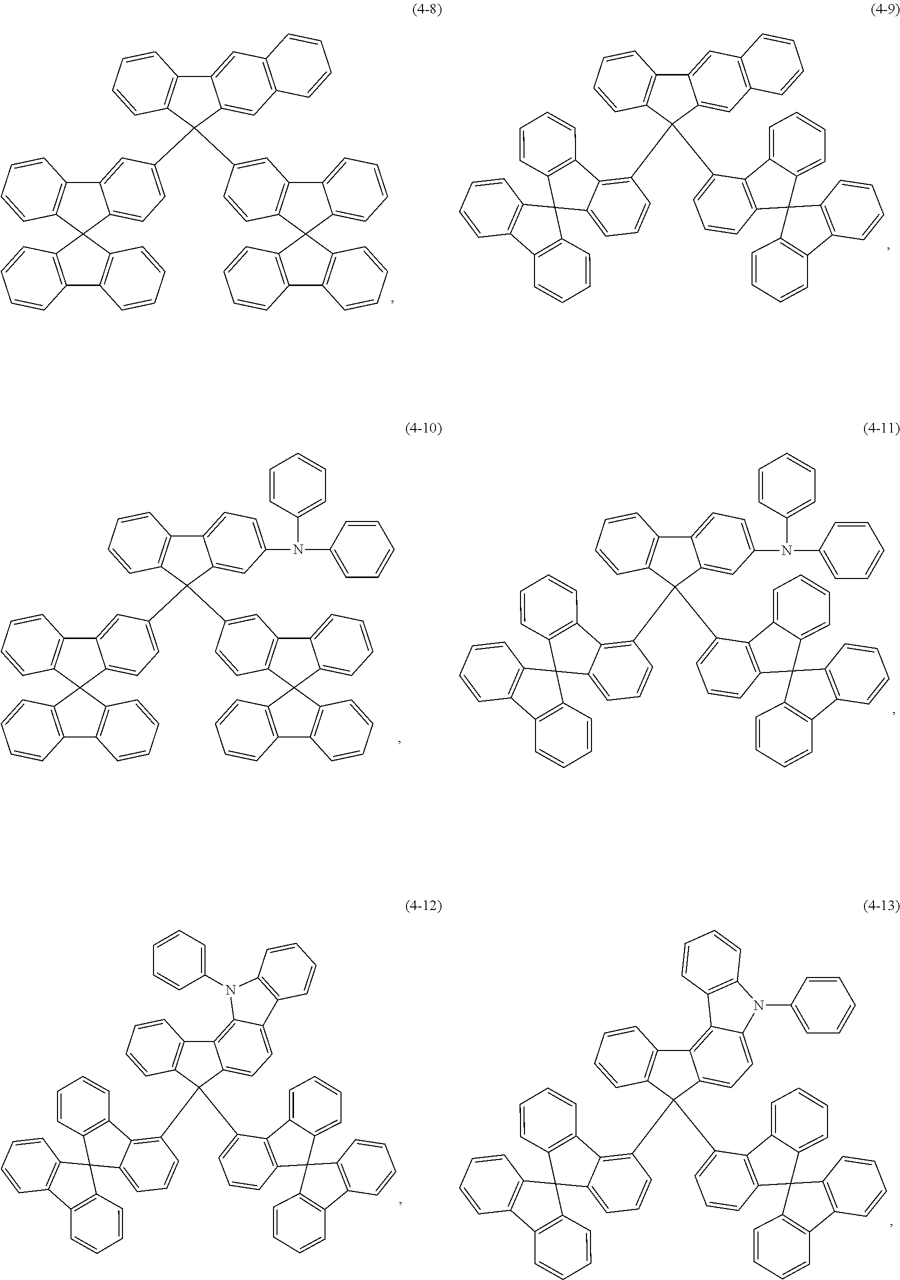

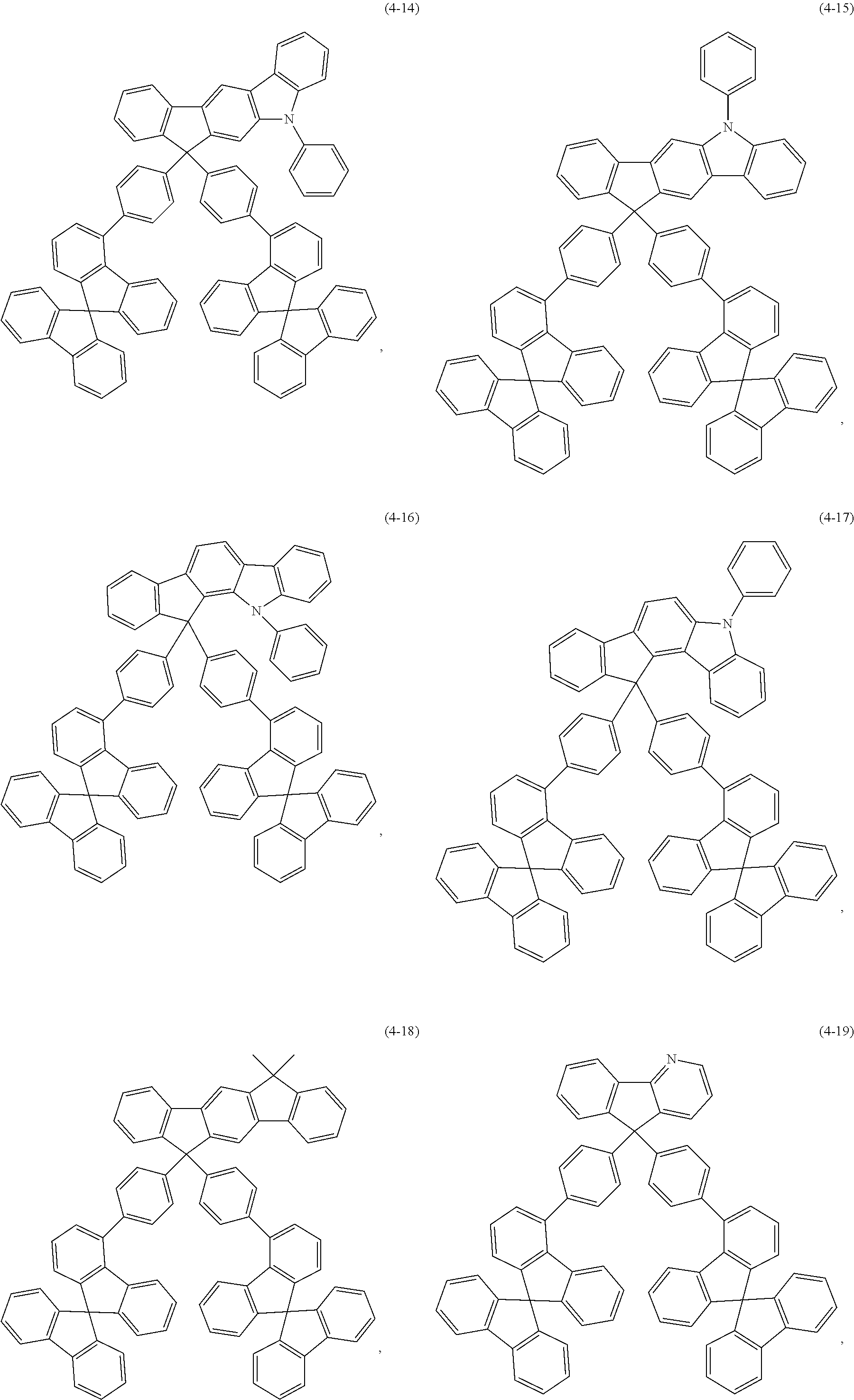

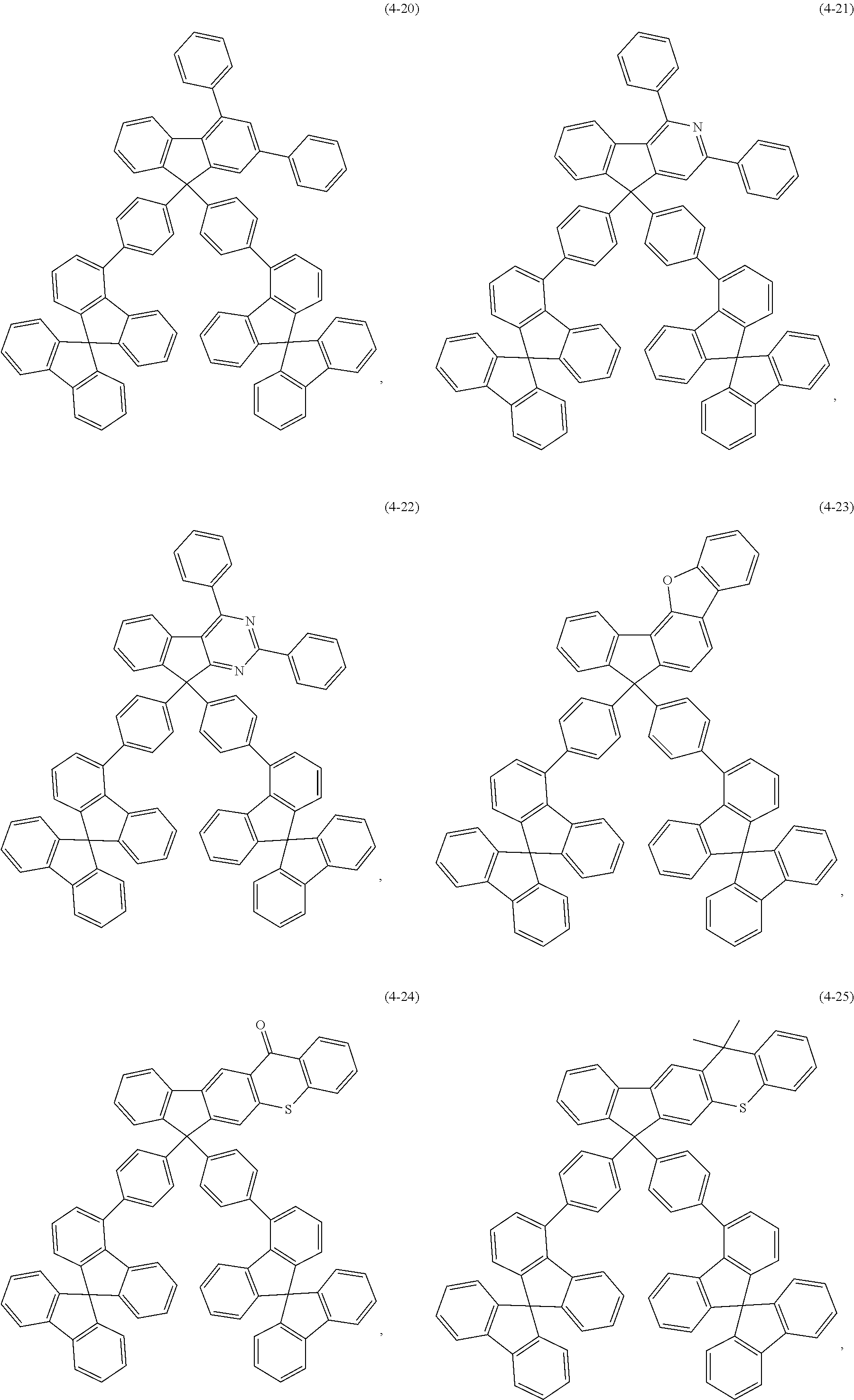

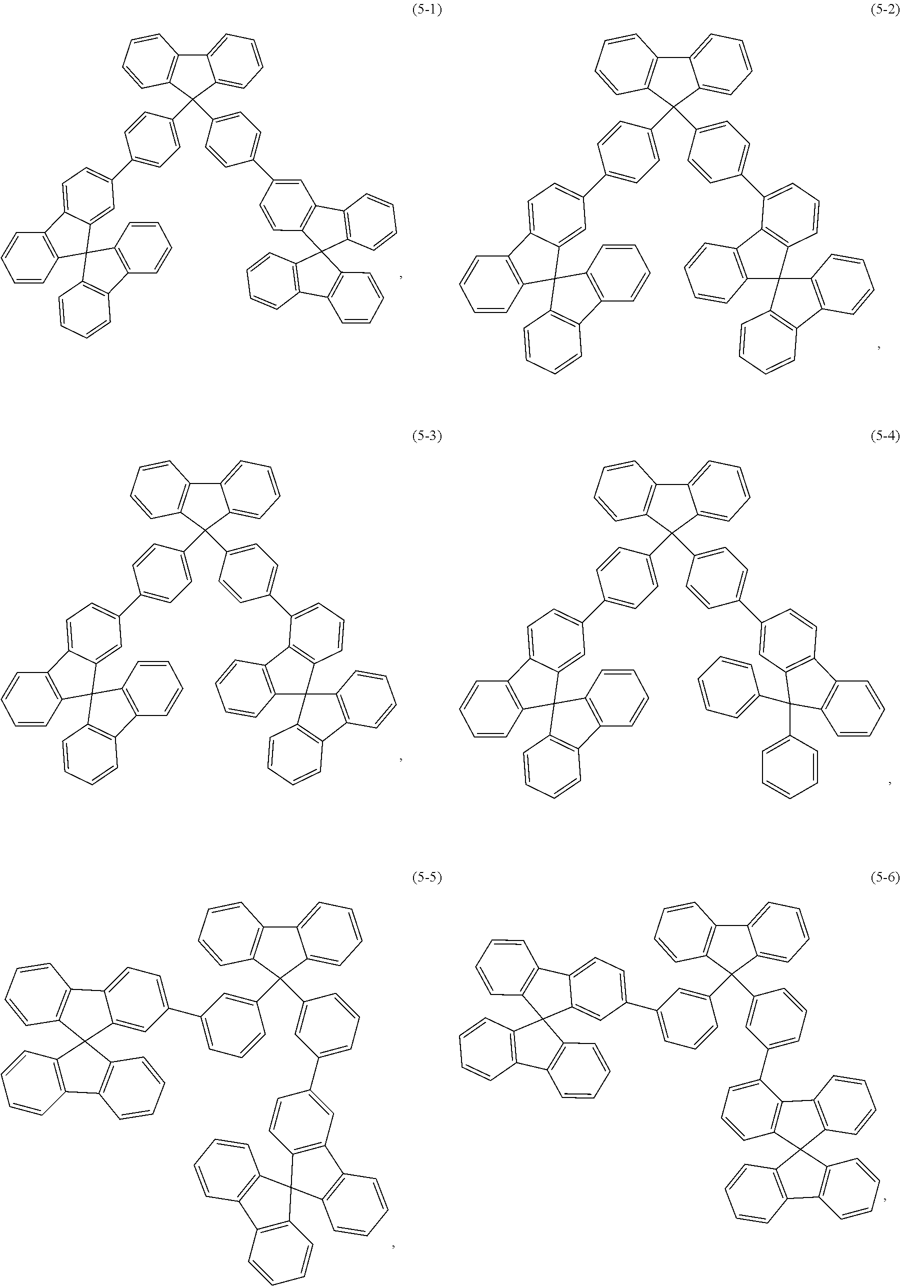

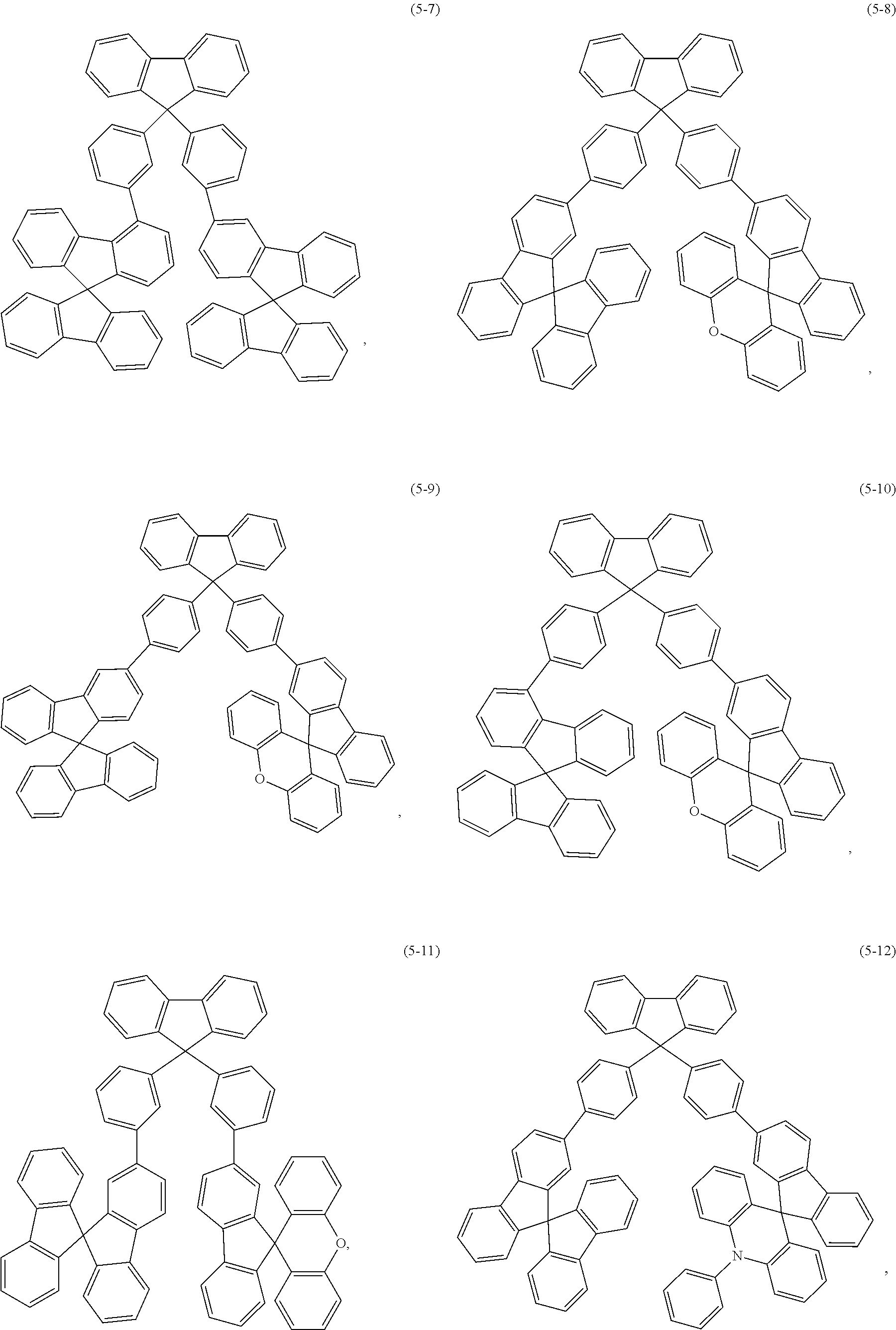

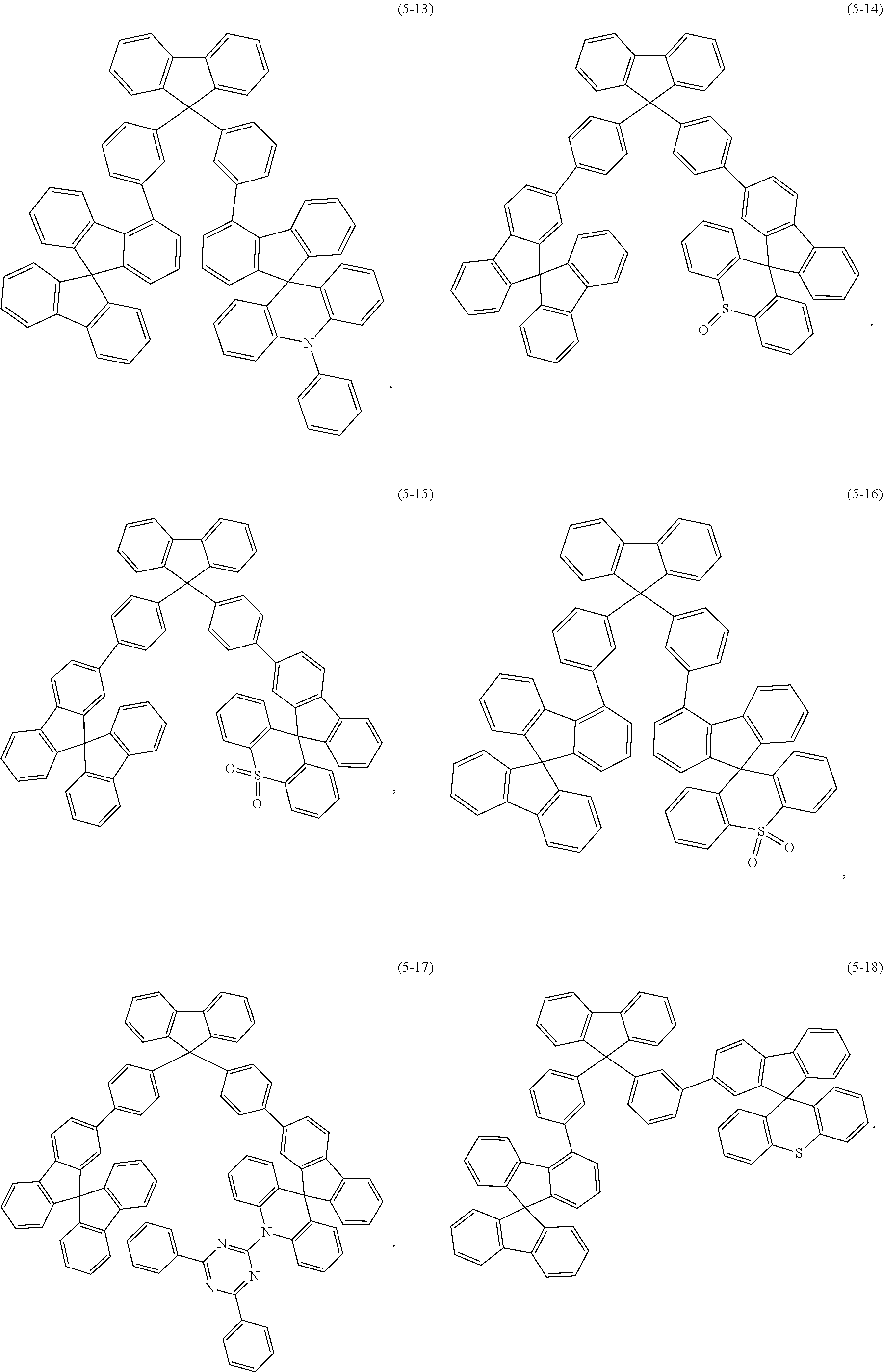

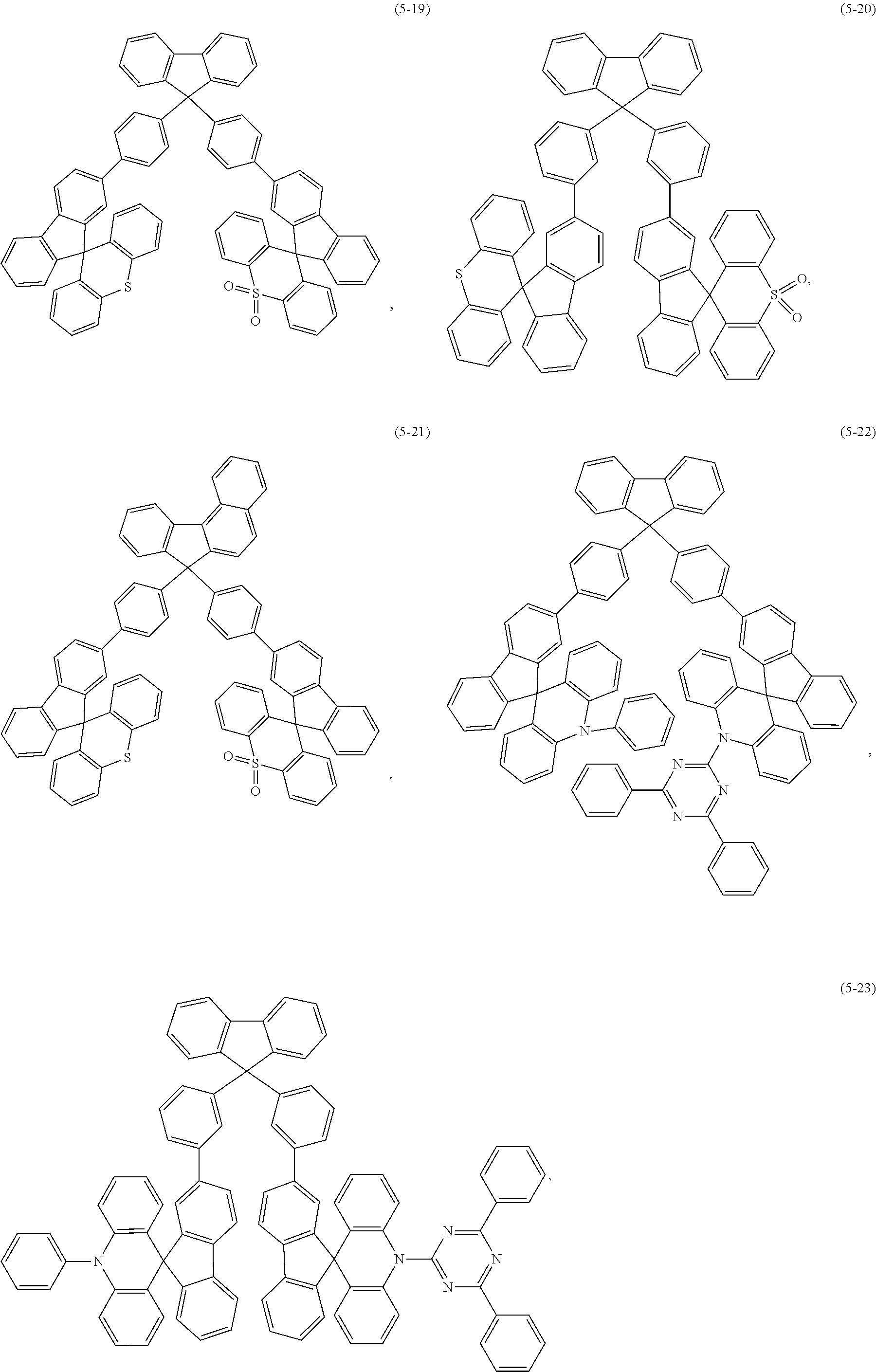

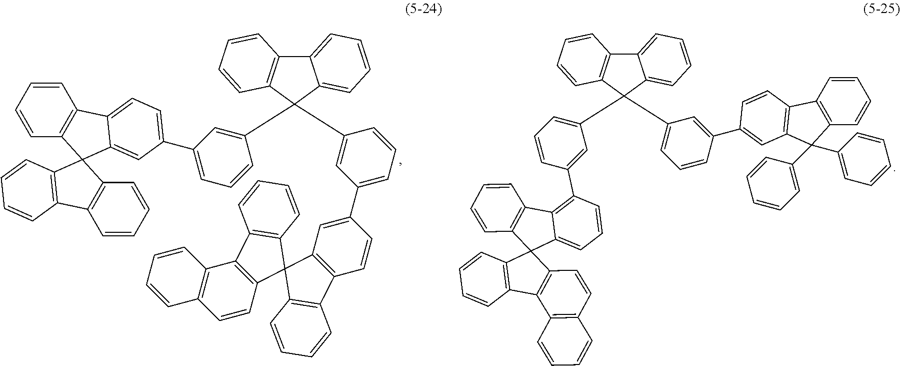

In a preferred embodiment, the spirocyclic derivative is a small molecular material.

As used herein, the term “small molecule” is not a polymer, but refers to a molecule of oligomer, dendrimer, or blend. In particular, there is no repetitive structure in small molecules. The molecular weight of the small molecule is no greater than 3000 g/mole, in a preferred embodiment, the molecular weight of the small molecule is no greater than 2000 g/mole, and in a more preferred embodiment, the molecular weight of the small molecule is no greater than 1500 g/mole.

Polymer includes homopolymer, copolymer, and block copolymer. In addition, in the present disclosure, the polymer also includes dendrimer. The synthesis and application of dendrimers are described in Dendrimers and Dendrons. Wiley-VCH Verlag GmbH & Co. KGaA, 2002, Ed George R. Newkome, Charles N. Moorefield, Fritz Vogtle.

Conjugated polymer is a polymer having a backbone is primarily consisted of the sp2 hybrid orbital of carbon (C) atom. Some known examples are polyacetylene and poly (phenylene vinylene), on the backbone of which the C atom can also be optionally substituted by other non-C atoms, and which is still considered to be a conjugated polymer when the sp2 hybridization on the backbone is interrupted by some natural defects. In addition, the conjugated polymer in the present disclosure may also comprise aryl amine, aryl phosphine and other heteroaromatics, organometallic complexes, and the like on the backbone.

The present disclosure further relates to a polymer, a repeating unit of the polymer comprises the above spirocyclic derivative.

In an embodiment, the polymer is a non-conjugated polymer, and the spirocyclic derivative is situated at a side chain of the polymer.

In another embodiment, the polymer is a conjugated polymer.

The disclosure further relates to a mixture comprising the spirocyclic derivative of the present disclosure, and an organic functional material.

The organic functional material includes: a hole (also called electron hole)-injection or transport material (HIM/HTM), a hole-blocking material (HBM), an electron-injection or transport material (EIM/ETM), an electron-blocking material (EBM), an organic matrix material (Host), a singlet emitter (fluorescent emitter), a thermally activated delayed fluorescent emitter (TADF), or a triplet emitter (phosphorescent emitter), in particular, light-emitting organometallic complex. Various organic functional materials are described in detail in, for example, WO2010135519A1, US20090134784A1, and WO2011110277A1, the entire contents of which are hereby incorporated by reference.

The organic functional material may be a small molecule or a polymer material.

In an embodiment, the mixture has the spirocyclic derivative in an amount of 50 wt % to 99.9 wt %, in a preferred embodiment, the mixture has the spirocyclic derivative in an amount of 60 wt % to 97 wt %, in a more preferred embodiment, the mixture has the spirocyclic derivative in an amount of 70 wt % to 95 wt %, and in a most preferred embodiment, the mixture has the spirocyclic derivative in an amount 70 wt % to 90 wt %.

In some embodiments, the mixture comprises the above spirocyclic derivative and a phosphorescent emitting material.

In some embodiments, the mixture comprises the above polymer and a phosphorescent emitting material.

In some embodiments, the mixture comprises the above spirocyclic derivative and a TADF material.

In some embodiments, the mixture comprises the above polymer and a TADF material.

In some embodiments, the mixture comprises the above spirocyclic derivative, a phosphorescent emitting material and a TADF material.

In some embodiments, the mixture comprises the above polymer, a phosphorescent emitting material and a TADF material.

In some embodiments, the mixture comprises the above spirocyclic derivative, and a fluorescent emitting material.

In some embodiments, the mixture comprises the above polymer, and a fluorescent emitting material.

In some embodiments, the mixture comprises the above spirocyclic derivative and a light-emitting quantum dot.

In some embodiments, the mixture comprises the above polymer and a light-emitting quantum dot.

The fluorescent emitting material or singlet emitter, phosphorescent emitting material or triplet emitter, TADF material and light-emitting quantum dot are described in more detail below (but not limited thereto).

1. Singlet Emitter

The singlet emitter tends to have a longer conjugate π-electron system. To date, there have been many examples, such as, but not limited to, styrylamine and derivatives thereof disclosed in JP2913116B and WO02001021729A1, and indenofluorene and derivatives thereof disclosed in WO2008/006449 and WO02007/140847.

In a preferred embodiment, the singlet emitter may be selected from the group consisting of monostyrylamines, distyrylamines, tristyrylamines, tetrastyrylamines, styrylphosphines, styryl ethers, and arylamines.

Mono styrylamine refers to a compound which comprises an unsubstituted or optionally substituted styryl group and at least one amine, most preferably an aromatic amine.

Distyrylamine refers to a compound comprising two unsubstituted or optionally substituted styryl groups and at least one amine, most preferably an aromatic amine.

Ternarystytylamine refers to a compound which comprises three unsubstituted or optionally substituted styryl groups and at least one amine, most preferably an aromatic amine.

Quaternary styrylamine refers to a compound comprising four unsubstituted or optionally substituted styryl groups and at least one amine, most preferably an aromatic amine.

In an embodiment, styrene is stilbene, which may be further optionally substituted.

The corresponding phosphines and ethers are defined similarly to amines.

Aryl amine or aromatic amine refers to a compound comprising three unsubstituted or optionally substituted aromatic cyclic or heterocyclic systems directly attached to nitrogen. In some embodiments, at least one of these aromatic cyclic or heterocyclic systems is selected from fused ring systems and in another embodiment, these aromatic cyclic or heterocyclic system has at least 14 aromatic ring atoms. In some preferred embodiments, aryl amine or aromatic amine are aromatic anthramine, aromatic anthradiamine, aromatic pyrene amines, aromatic pyrene diamines, aromatic chrysene amines and aromatic chrysene diamine. Aromatic anthramine refers to a compound in which a diarylamino group is directly attached to anthracene, most preferably at position 9. Aromatic anthradiamine refers to a compound in which two diarylamino groups are directly attached to anthracene, most preferably at positions 9, 10. Aromatic pyrene amines, aromatic pyrene diamines, aromatic chrysene amines and aromatic chrysene diamine are similarly defined, wherein the diarylarylamino group is most preferably attached to position 1 or 1 and 6 of pyrene.

Examples of singlet emitter based on vinylamine and arylamine are also examples which may be found in the following patent documents: WO 2006/000388, WO 2006/058737, WO 2006/000389, WO 2007/065549, WO 2007/115610, U.S. Pat. No. 7,250,532 B2, DE 102005058557 A1, CN 1583691 A, JP 08053397 A, U.S. Pat. No. 6,251,531 B1, US 2006/210830 A, EP 1957606 A1, and US 2008/0113101 A1, the whole contents of which are incorporated herein by reference.

Examples of singlet light emitters based on distyrylbenzene and its derivatives may be found in, for example, U.S. Pat. No. 5,121,029.

Further singlet emitters may be selected from the group consisting of: indenofluorene-amine and indenofluorene-diamine such as disclosed in WO 2006/122630, benzoindenofluorene-amine and benzoindenofluorene-diamine such as disclosed in WO 2008/006449, dibenzoindenofluorene-amine and dibenzoindenofluorene-diamine such as disclosed in WO2007/140847.

Other materials useful as singlet emitters include, but not limited to, polycyclic aromatic compounds, especially any one selected from the derivatives of the following compounds: anthracenes such as 9,10-di-naphthylanthracene, naphthalene, tetraphenyl, oxyanthene, phenanthrene, perylene (such as 2,5,8,11-tetra-t-butylatedylene), indenoperylene, phenylenes (such as 4,4′-(bis(9-ethyl-3-carbazovinylene)-1,1′-biphenyl), periflanthene, decacyclene, coronene, fluorene, spirobifluorene, arylpyren (e.g., US20060222886), arylenevinylene (e.g., U.S. Pat. No. 5,121,029, U.S. Pat. No. 5,130,603), cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds (US 2007/0092753 A1), bis (azinyl) methene compounds, carbostyryl compounds, oxazone, benzoxazole, benzothiazole, benzimidazole, and diketopyrnolopyrrole. Examples of some singlet emitter materials may be found in the following patent documents: US 20070252517 A1, U.S. Pat. No. 4,769,292, U.S. Pat. No. 6,020,078, US 2007/0252517 A1, US 2007/0252517 A1, the whole contents of which are incorporated herein by reference.

Examples of suitable singlet emitters are listed below:

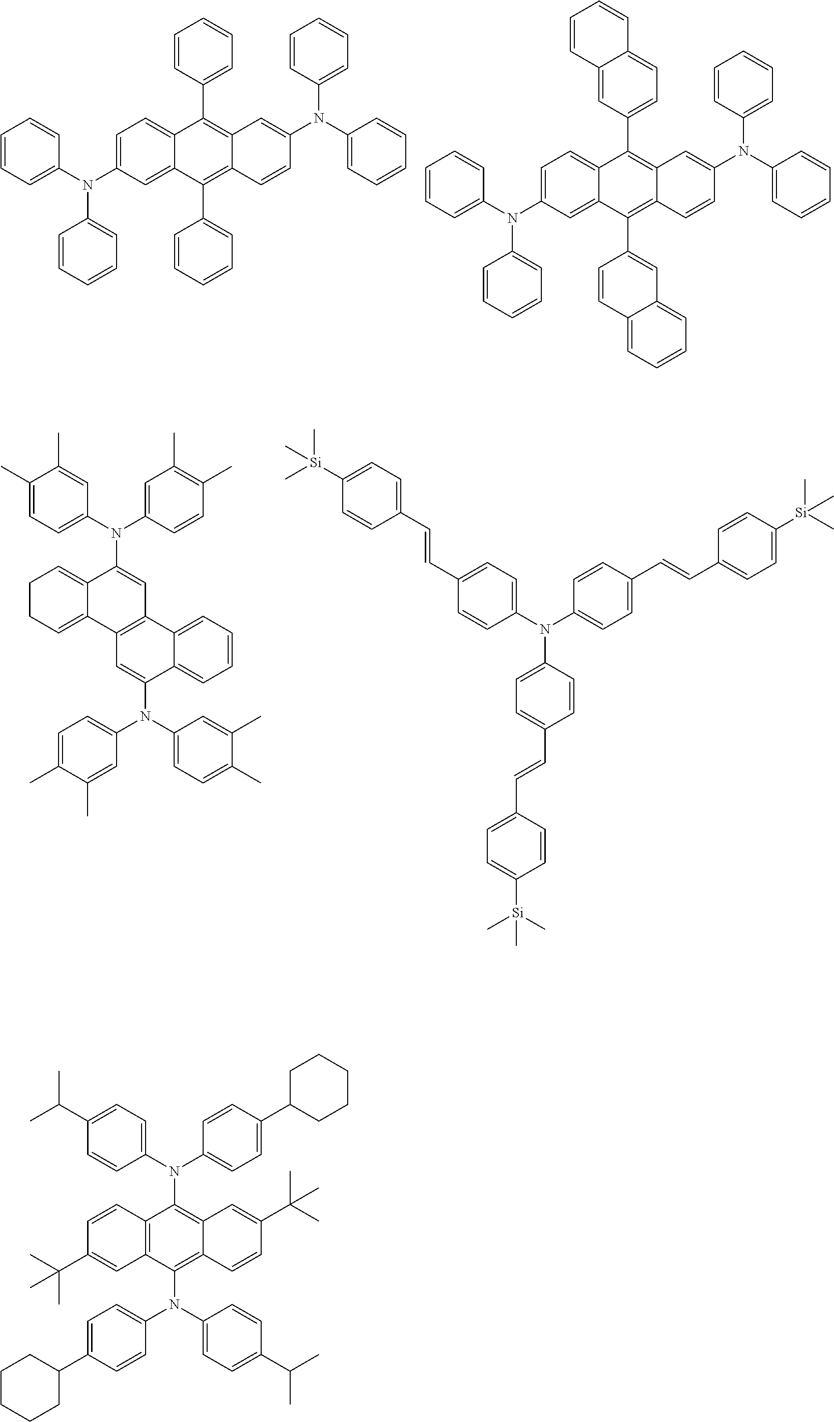

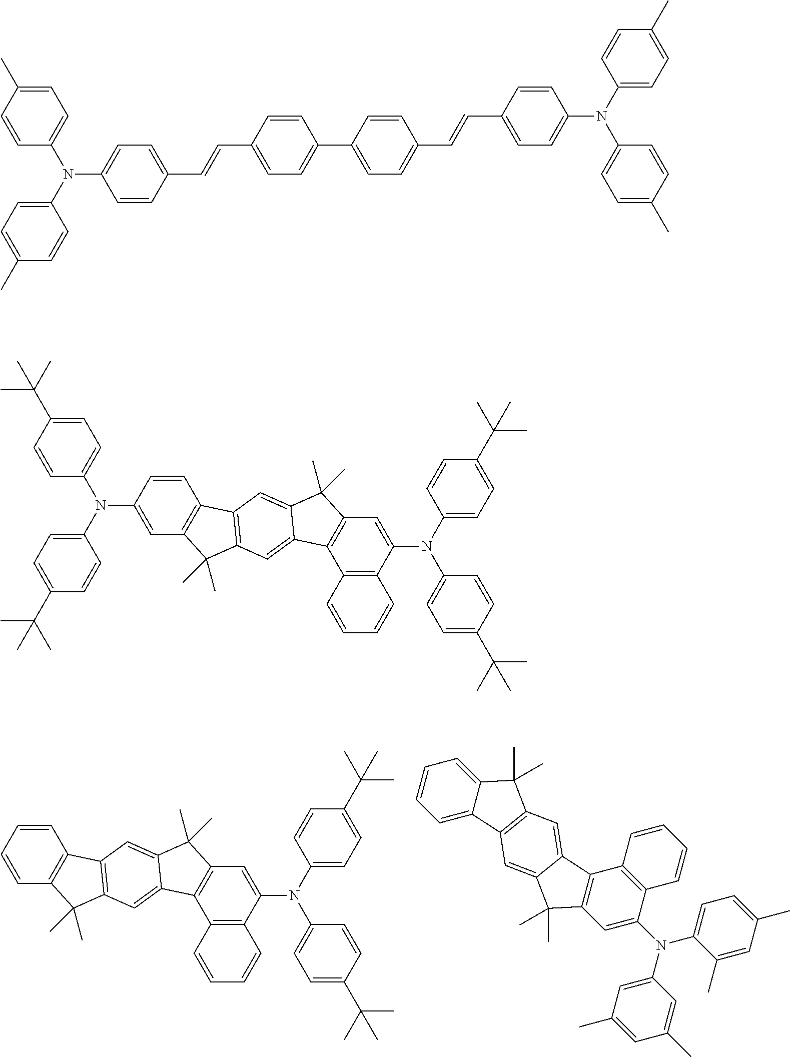

2. Thermally Activated Delayed Fluorescent Material (TADF):

Traditional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphor materials are expensive, the material stability is poor, and the device efficiency roll-off is a serious problem, which limit its application in OLED. Thermally-activated delayed fluorescent materials are the third generation of organic light-emitting materials developed after organic fluorescent materials and organic phosphorescent materials. This type of material generally has a small singlet-triplet energy level difference (ΔEst), and triplet excitons can be converted to singlet excitons by intersystem crossing. This can make full use of the singlet excitons and triplet excitons formed under electric excitation. The device can achieve 100% quantum efficiency.

The TADF material needs to have a small singlet-triplet energy level difference, typically ΔEst<0.3 eV in some embodiments. ΔEst<0.2 eV, in some preferred embodiments ΔEst<0.1 eV, and in a specific embodiment ΔEst<0.05 eV. In a preferred embodiment. TADF has good fluorescence quantum efficiency. Some TADF emitting materials can be found in the following patent documents: CN103483332(A), TW201309696(A), TW201309778(A), TW201343874(A), TW201350558(A), US20120217869(A1), WO2013133359(A1), WO2013154064 (A1), Adachi, et. al. Adv. Mater., 21, 2009, 4802. Adachi, et. al. Appl. Phys. Lett., 98, 2011, 083302. Adachi, et. al. Appl. Phys. Lett., 101, 2012, 093306. Adachi, et. al. Chem. Commun., 48, 2012, 11392, Adachi, et. al. Nature Photonics, 6, 2012, 253, Adachi, et. al. Nature, 492, 2012, 234. Adachi. et. al. J. Am. Chem. Soc. 134, 2012, 14706. Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi. et. al. Chem. Mater., 25, 2013, 3766. Adachi, et. Al. J. Mater. Chem. C., 1, 2013, 4599, Adachi, et. al. J. Phys. Chem. A., 117, 2013, 5607. The entire contents of the above listed patent or literature documents are hereby incorporated by reference.

Some examples of suitable TADF light-emitting materials are listed in the following table:

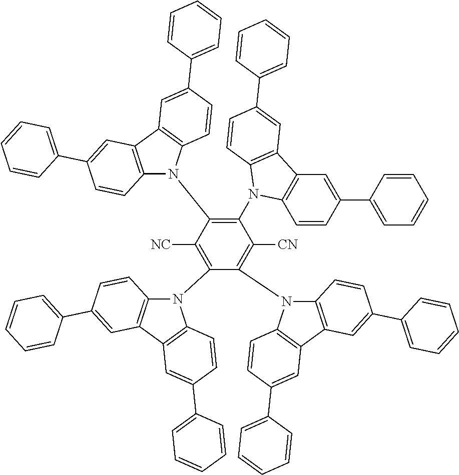 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21]

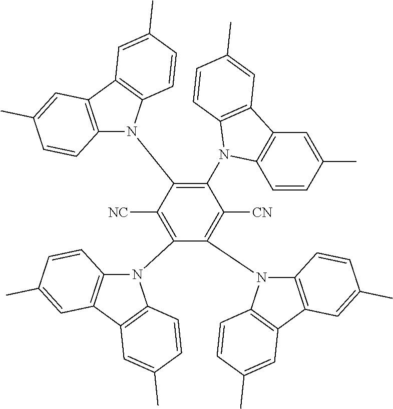

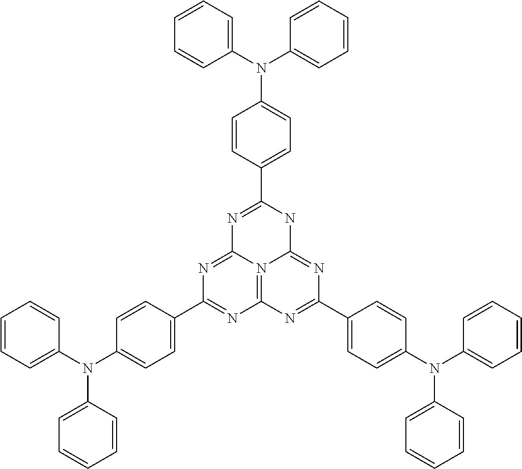

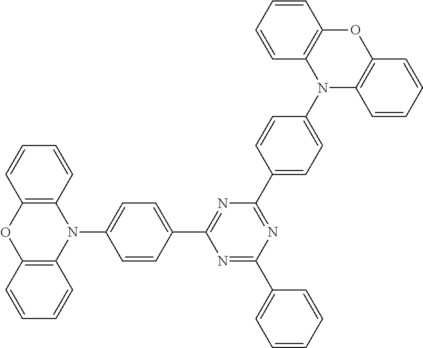 [SMILES: c1ccc(-c2nc(-c3ccc(N4c5ccccc5Oc5ccccc54)cc3)nc(-c3ccc(N4c5ccccc5Oc5ccccc54)cc3)n2)cc1]

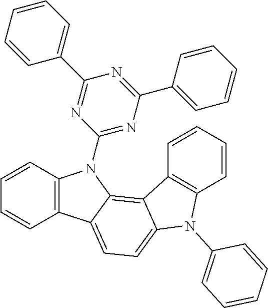 [SMILES: c1ccc(-c2nc(-c3ccccc3)nc(-n3c4ccccc4c4ccc5c(c6ccccc6n5-c5ccccc5)c43)n2)cc1]

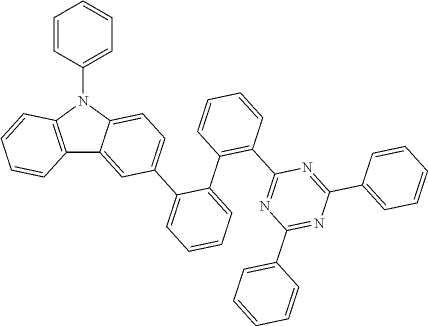 [SMILES: c1ccc(-c2nc(-c3ccccc3)nc(-c3ccccc3-c3ccccc3-c3ccc4c(c3)c3ccccc3n4-c3ccccc3)n2)cc1]

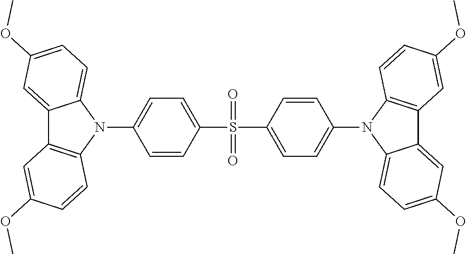 [SMILES: COc1ccc2c(c1)c1cc(OC)ccc1n2-c1ccc(S(=O)(=O)c2ccc(-n3c4ccc(OC)cc4c4cc(OC)ccc43)cc2)cc1]

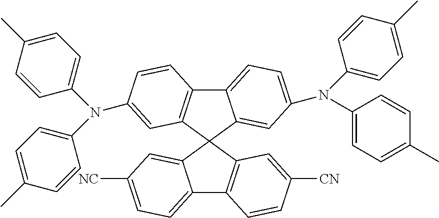

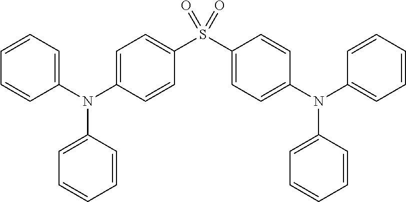 [SMILES: O=S(=O)(c1ccc(N(c2ccccc2)c2ccccc2)cc1)c1ccc(N(c2ccccc2)c2ccccc2)cc1]

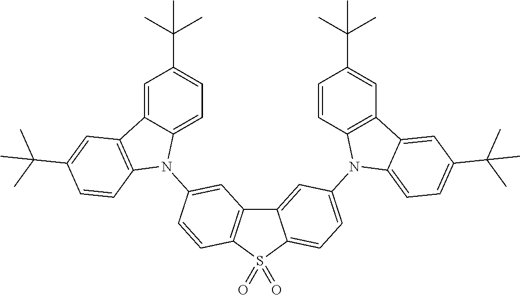 [SMILES: CC(C)(C)c1ccc2c(c1)c1cc(C(C)(C)C)ccc1n2-c1ccc2c(c1)-c1cc(-n3c4ccc(C(C)(C)C)cc4c4cc(C(C)(C)C)ccc43)ccc1S2(=O)=O]

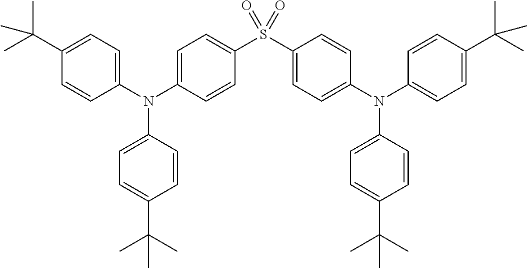 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

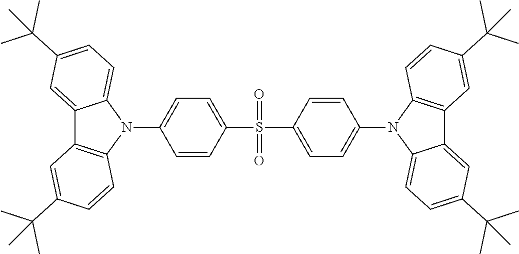 [SMILES: CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)cc2)cc1]

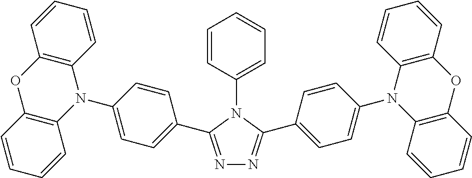 [SMILES: c1ccc(-n2c(-c3ccc(N4c5ccccc5Oc5ccccc54)cc3)nnc2-c2ccc(N3c4ccccc4Oc4ccccc43)cc2)cc1]

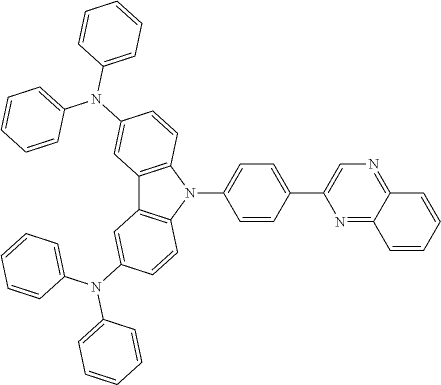 [SMILES: c1ccc(N(c2ccccc2)c2ccc3c(c2)c2cc(N(c4ccccc4)c4ccccc4)ccc2n3-c2ccc(-c3cnc4ccccc4n3)cc2)cc1]

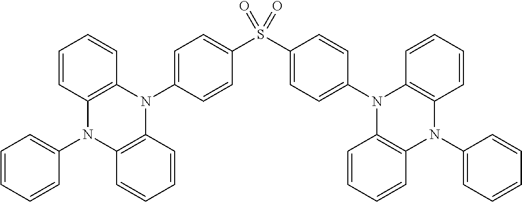 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

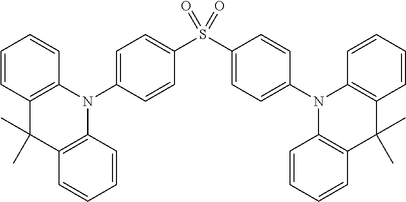 [SMILES: CC1(C)c2ccccc2N(c2ccc(S(=O)(=O)c3ccc(N4c5ccccc5C(C)(C)c5ccccc54)cc3)cc2)c2ccccc21]

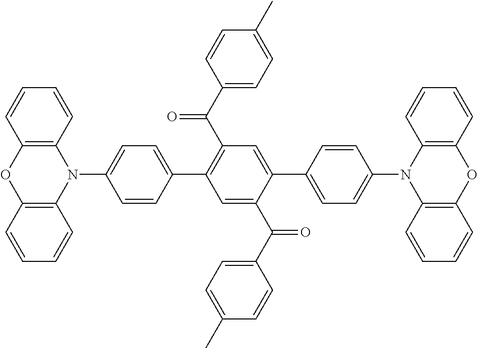 [SMILES: Cc1ccc(C(=O)c2cc(-c3ccc(N4c5ccccc5Oc5ccccc54)cc3)c(C(=O)c3ccc(C)cc3)cc2-c2ccc(N3c4ccccc4Oc4ccccc43)cc2)cc1]

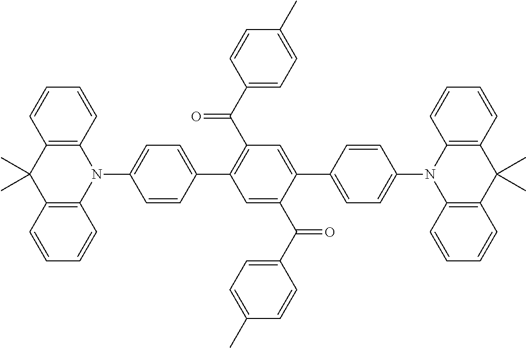

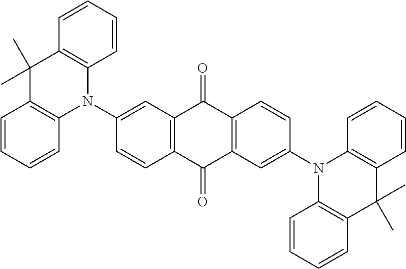 [SMILES: CC1(C)c2ccccc2N(c2ccc3c(c2)C(=O)c2ccc(N4c5ccccc5C(C)(C)c5ccccc54)cc2C3=O)c2ccccc21]

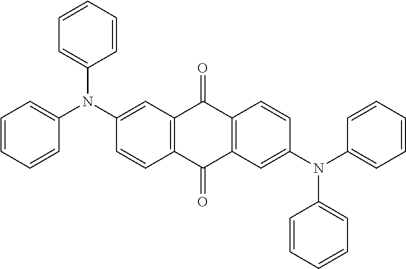 [SMILES: O=C1c2ccc(N(c3ccccc3)c3ccccc3)cc2C(=O)c2ccc(N(c3ccccc3)c3ccccc3)cc21]

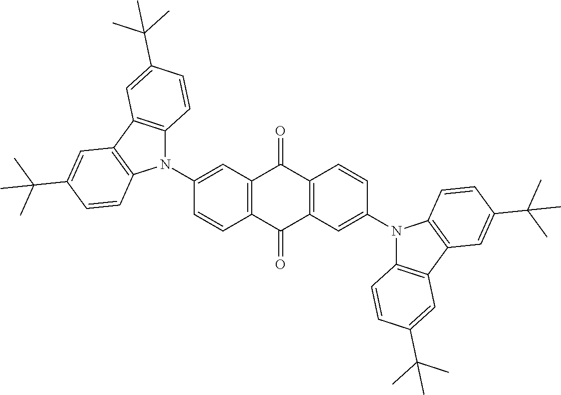 [SMILES: CC(C)(C)c1ccc2c(c1)c1cc(C(C)(C)C)ccc1n2-c1ccc2c(c1)C(=O)c1ccc(-n3c4ccc(C(C)(C)C)cc4c4cc(C(C)(C)C)ccc43)cc1C2=O]

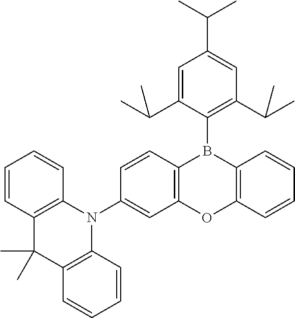 [SMILES: CC(C)c1cc(C(C)C)c(B2c3ccccc3Oc3cc(N4c5ccccc5C(C)(C)c5ccccc54)ccc32)c(C(C)C)c1]

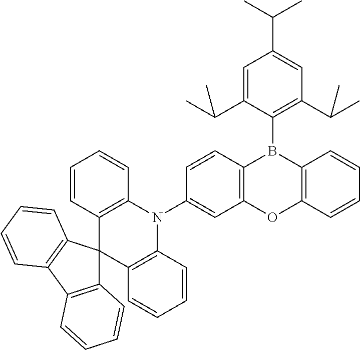 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

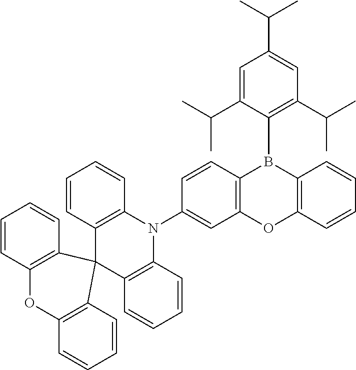 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

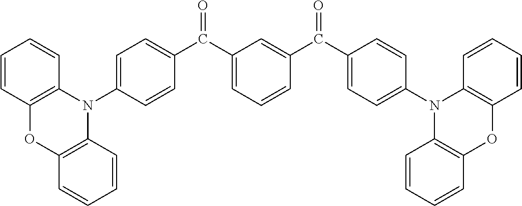 [SMILES: O=C(c1ccc(N2c3ccccc3Oc3ccccc32)cc1)c1cccc(C(=O)c2ccc(N3c4ccccc4Oc4ccccc43)cc2)c1]

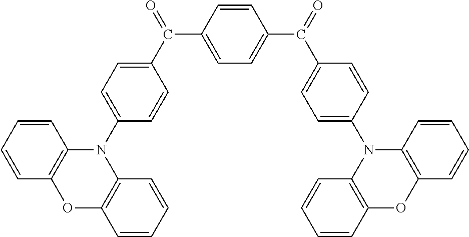 [SMILES: O=C(c1ccc(C(=O)c2ccc(N3c4ccccc4Oc4ccccc43)cc2)cc1)c1ccc(N2c3ccccc3Oc3ccccc32)cc1]

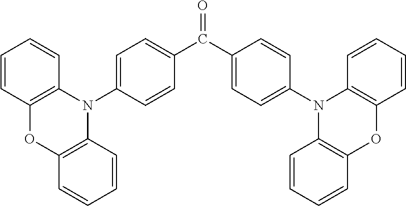 [SMILES: O=C(c1ccc(N2c3ccccc3Oc3ccccc32)cc1)c1ccc(N2c3ccccc3Oc3ccccc32)cc1]

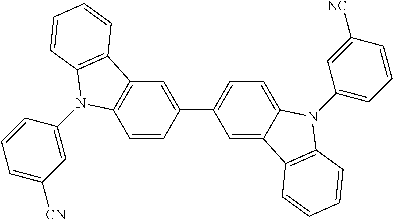 [SMILES: N#Cc1cccc(-n2c3ccccc3c3cc(-c4ccc5c(c4)c4ccccc4n5-c4cccc(C#N)c4)ccc32)c1]

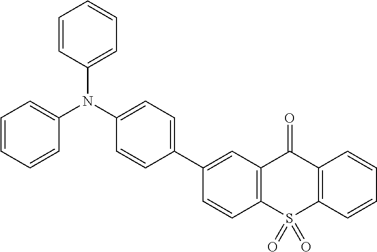 [SMILES: O=C1c2ccccc2S(=O)(=O)c2ccc(-c3ccc(N(c4ccccc4)c4ccccc4)cc3)cc21]

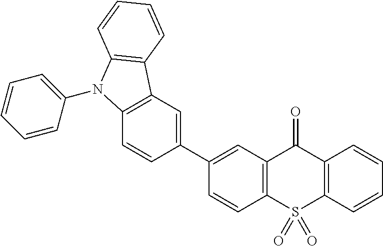 [SMILES: O=C1c2ccccc2S(=O)(=O)c2ccc(-c3ccc4c(c3)c3ccccc3n4-c3ccccc3)cc21]

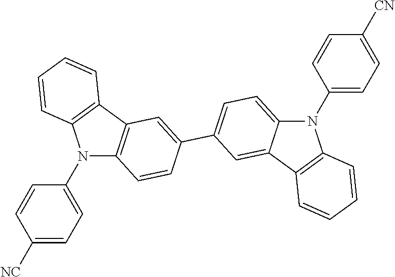 [SMILES: N#Cc1ccc(-n2c3ccccc3c3cc(-c4ccc5c(c4)c4ccccc4n5-c4ccc(C#N)cc4)ccc32)cc1]

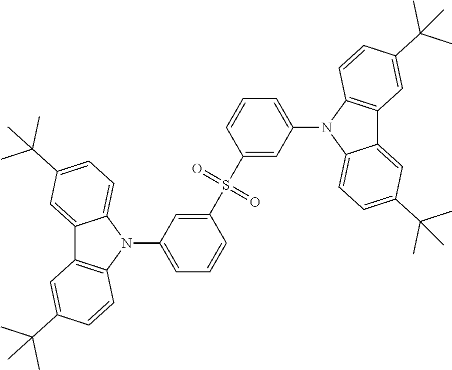 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

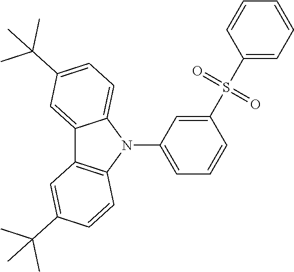 [SMILES: CC(C)(C)c1ccc2c(c1)c1cc(C(C)(C)C)ccc1n2-c1cccc(S(=O)(=O)c2ccccc2)c1]

3. Triplet Emitter

The triplet emitter is also called a phosphorescent emitter. In a preferred embodiment, the triplet emitter is a metal complex of the general formula M(L)n, wherein M is a metal atom; L may be the same or different ligand each time it is present, and is bonded or coordinated to the metal atom M at one or more positions; n is an integer greater than 1, and in some embodiments, n is 1, 2, 3, 4, 5 or 6. Alternatively, these metal complexes are attached to a polymer by one or more positions, particularly y through an organic ligand.

In a preferred embodiment the metal atom M is selected from the group consisting of transition metal elements or lanthanides or actinides. In some preferred embodiments. M is selected from the group consisting of Ir, Pt, Pd, Au, Rh, Ru, Os, Sm, Eu, Gd, Th, Dy, Re, Cu or Ag. In some particularly preferred embodiments, M is selected from the group consisting of Os, Ir, Ru, Rh, Re, Pd, or Pt.

In some embodiments, the triplet emitter comprises a chelating ligand, i.e., a ligand, coordinated to the metal by at least two bonding sites, and it is particularly preferred that the triplet emitter comprises two or three identical or different bidentate or multidentate ligand. Chelating ligands help to improve stability of metal complexes.

Examples of organic ligands may be selected from the group consisting of phenylpyridine derivatives, 7,8-benzoquinoline derivatives, 2 (2-thienyl) pyridine derivatives, 2 (1-naphthyl) pyridine derivatives, or 2 phenylquinoline derivatives. All of these organic ligands may be optionally substituted, for example, optionally substituted with fluoromethyl or trifluoromethyl. The auxiliary ligand may be selected from acetylacetonate or picric acid.

In a preferred embodiment, the metal complex which may be used as the triplet emitter may have the following form

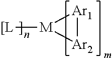

wherein M is a metal element selected from transition metal elements, lanthanides or actinides:

Ar₁ may be the same or different cyclic group each time it is present, which comprises at least one donor atom (that is, an atom with a lone pair of electrons, such as nitrogen atom or phosphorus atom), which is coordinated to M through the donor atom:

Ar₂ may be the same or different cyclic group comprising at least one C atom and is coordinated to M through the C atom:

Ar₁ and Ar₂ are covalently bonded together, wherein each of them may carry one or more substituents which may also be joined together by substituents;

L may be the same or different at each occurrence and is an auxiliary ligand, in a preferred embodiment, L is a bidentate chelating ligand, and in a most preferred embodiment, L is a monoanionic bidentate chelating ligand;

m is 1, 2 or 3, in some preferred embodiments, m is 2 or 3, and particularly m is 3; and

n is 0, 1, or 2, in some preferred embodiments n is 0 or 1, particularly n is 0.

Examples of triplet emitter materials and examples of applications thereof may be found in the following patent documents and references: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728, WO 2010086089, WO 2010099852, WO 2010102709, US 20070087219 A1, US 20090061681 A1, US 20010053462 A1, Baldo, Thompson et al. Nature 403, (2000), 750-753, US 20090061681 A1, US 20090061681 A1, Adachi et al. Appl. Phys. Lett. 78 (2001), 1622-1624. J. Kido et al. Appl. Phys. Lett. 65 (1994), 2124. Kido et al. Chem. Lett. 657, 1990. US 2007/0252517 A1, Johnson et al., JACS 105, 1983, 1795, Wrighton. JACS 96, 1974, 998, Ma et al., Synth. Metals 94, 1998, 245, U.S. Pat. No. 6,824,895, U.S. Pat. No. 7,029,766, U.S. Pat. No. 6,835,469, U.S. Pat. No. 6,830,828, US 20010053462 A1, WO 2007095118 A1, US 2012004407A1, WO 2012007088A1, WO02012007087A1, WO 2012007086A1, US 2008027220A1, WO 2011157339A1, CN 102282150A, and WO 2009118087A1. The entire contents of the above listed patent or literature documents are hereby incorporated by reference.

Examples of suitable triplet emitter are given in the following table:

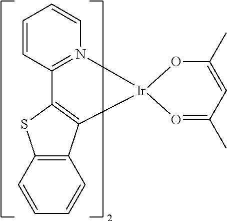

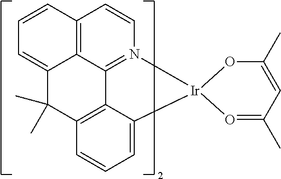

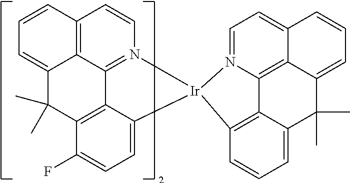

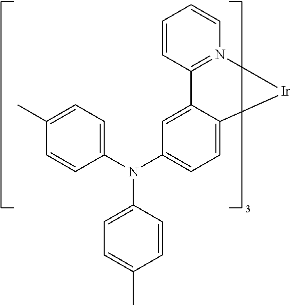

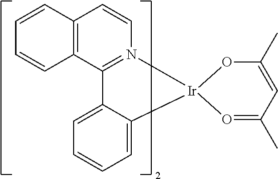

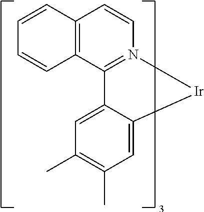

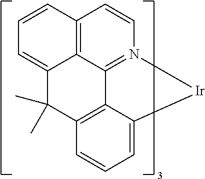

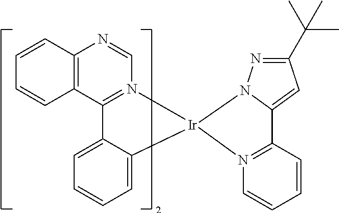

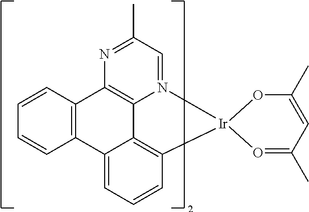

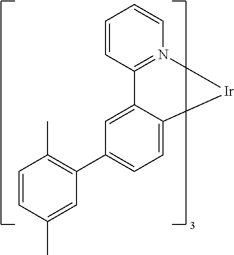

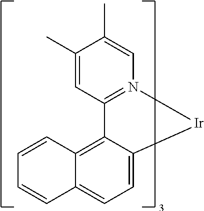

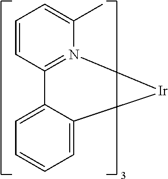

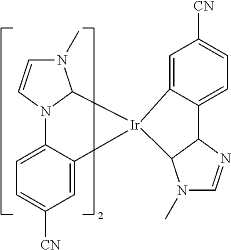

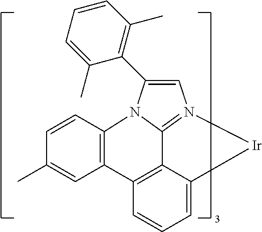

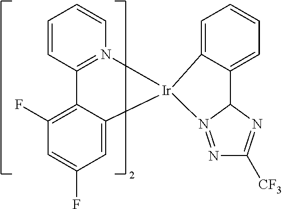

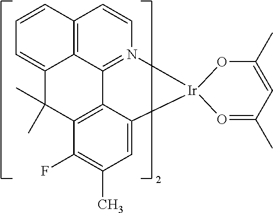

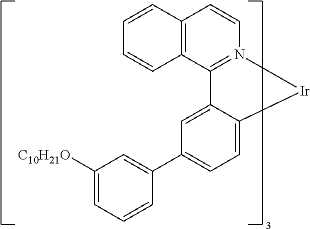

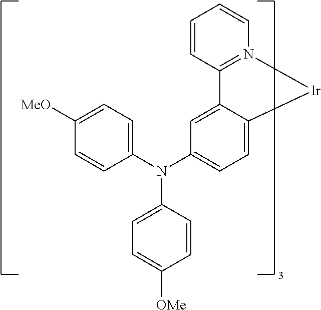

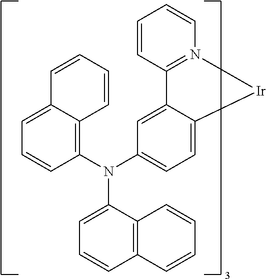

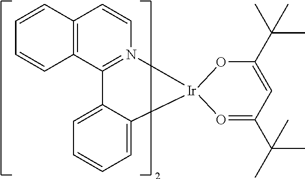

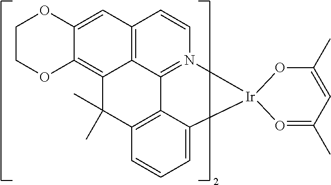

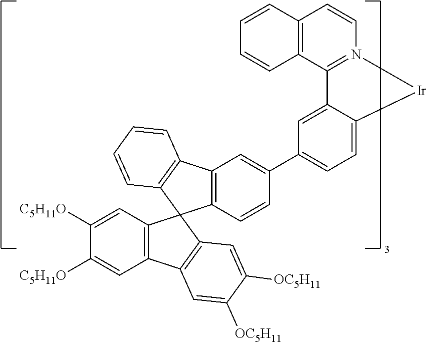

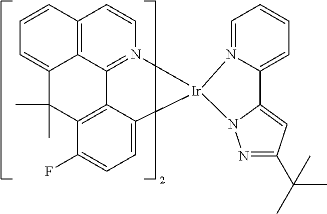

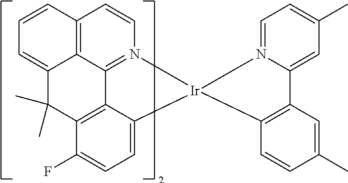

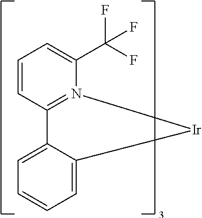

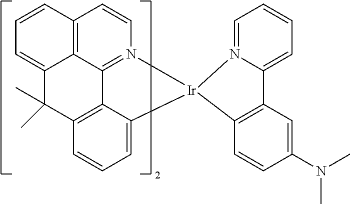

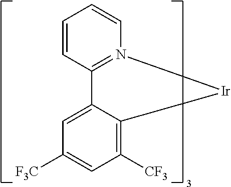

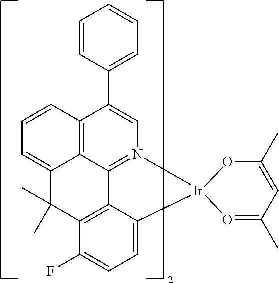

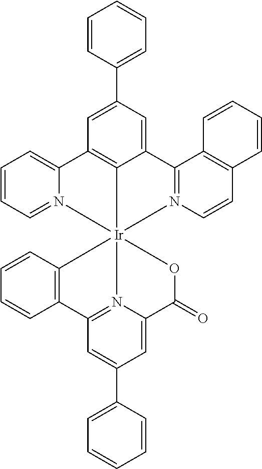

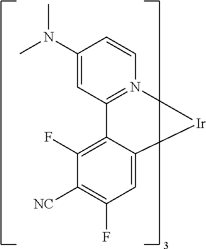

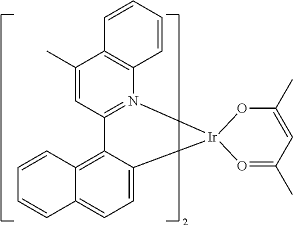

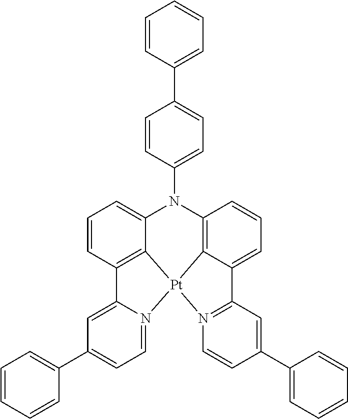

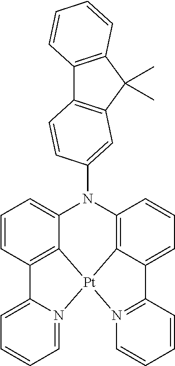

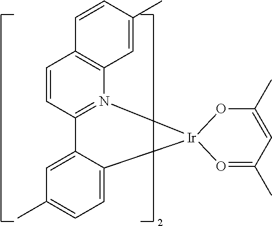

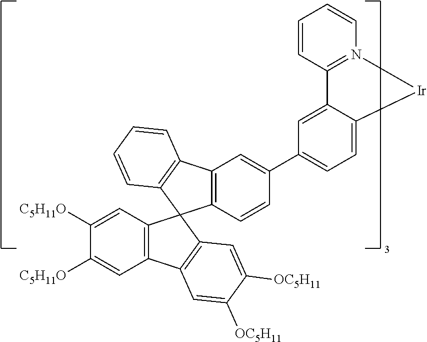

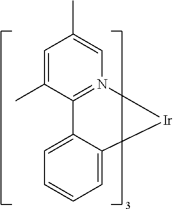

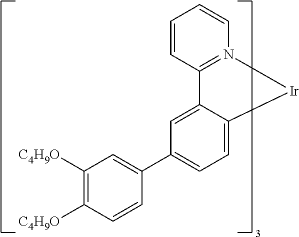

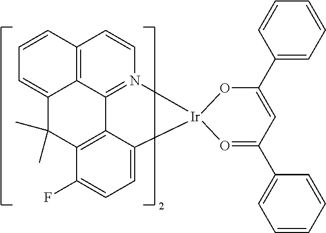

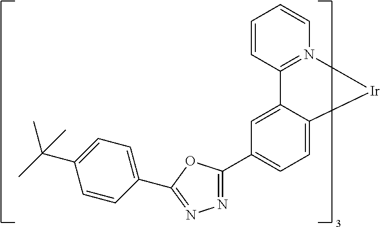

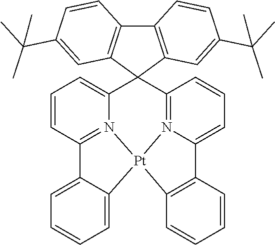

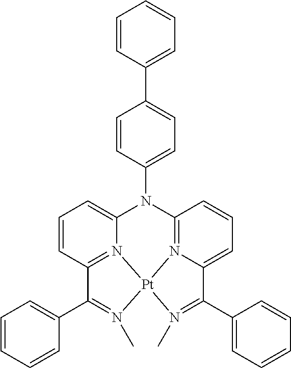

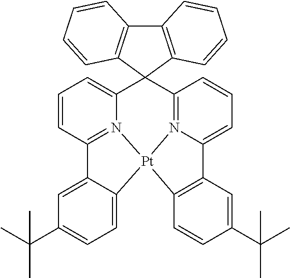

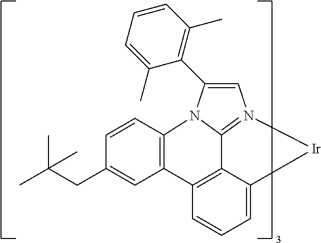

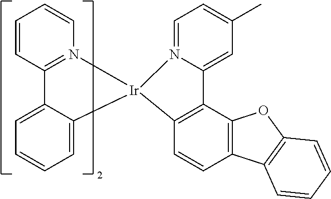

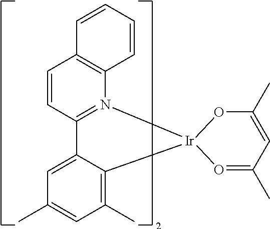

4. Light-Emitting Quantum Dots

In general light-emitting quantum dots can emit light at a wavelength of 380 nanometers to 2500 nanometers. For example, it has been found that the quantum dots with a CdS core have an emission wavelength in the range of about 400 nm to 560 nm; the quantum dots with a CdSe core have an emission wavelength in the range of about 490 nm to 620 nm: the quantum dots with CdTe cores have an emission wavelength in the range of about 620 nanometers to 680 nanometers: the quantum dots with a InGaP core have an emission wavelength in the range of about 600 nanometers to 700 nanometers; the quantum dots with a PbS core have an emission wavelength in the range of about 800 nanometers to 2500 nanometers; the quantum dots with a PbSe core have an emission wavelength in the range of about 1200 nm to 2500 nm; the quantum dots with a CuInGaS core have an emission wavelength in the range of about 600 nm to 680 nm; the quantum dots with a ZnCuInGaS core have an emission wavelength in the range of about 500 nm to 620 nm; and the quantum dot with a CuInGaSe core have an emission wavelength in the range of about 700 nm to 1000 nm.

In some embodiments, the quantum dot material includes at least one emitting blue light with a peak luminous wavelength of 450 nm to 460 nm, or green light with a peak luminous wavelength of 520 nm to 540 nm, or red light with a peak luminous wavelength of 615 nm to 630 nm, or their mixture.

Quantum dots included may be selected for particular chemical compositions, topographical structures, and/or size dimensions to obtain light that emits a desired wavelength under electrical stimulation. The relationship between the luminescent properties of quantum dots and their chemical composition, morphology structure and/or size can be found in Annual Review of Material Sci., 2000, 30, 545-610; Optical Materials Express., 2012, 2, 594-628; and Nano Res. 2009, 2, 425-447. The entire contents of the above listed patent documents are hereby incorporated by reference.

The narrow particle size distribution of quantum dots enables them to have a narrower luminescence spectrum (J. Am. Chem. Soc., 1993, 115, 8706: and US 20150108405). In addition, depending on the various chemical composition and structure used, the size of the quantum dots needs to be adjusted within the above-mentioned size range to obtain the luminescent properties of the desired wavelength.

In some embodiments, the light-emitting quantum dots are semiconductor nanocrystals. In an embodiment, the size of the semiconductor nanocrystals is in the range of about 5 nanometers to about 15 nanometers. In addition, depending on the various chemical composition and structure used, the size of the quantum dots needs to be adjusted within the above-mentioned size range to obtain the luminescent properties of the desired wavelength.

The semiconductor nanocrystal includes at least one semiconductor material, wherein the semiconductor material may be selected from binary or polybasic semiconductor compounds of Group IV, II-VI, II-V, III-V, III-VI, IV-VI, I-III-VI, II-IV-VI, and II-IV-V of the periodic table, or their mixtures. Examples of specific semiconductor materials include, but are not limited to: Group IV semiconductor compounds, including elemental Si, Ge and binary compounds SiC. SiGe; Group II-VI semiconductor compounds, including: binary compounds including CdSe, CdTe, CdO, CdS, CdSe, ZnS, ZnSe, ZnTe, ZnO, HgO, HgS, HgSe, and HgTe, ternary compounds including CdSeS, CdSeTe, CdSTe, CdZnS, CdZnSe, CdZnTe, CgHgS, CdHgSe, ZnSeS, ZnSeTe, ZnSbe, HgSeS, HgSeTe, HgSTe, HgZnS, and HgSeSe, and quaternary compounds including CgHgSeS, CdHgSeTe, CgHgSTe, CdZnSeS, CdZnSeTe, HgZnSeTe, HgZnSTe, CdZnSTe, and HgZnSeS; Group II-V semiconductor compounds including: binary compounds including AlN, AlP, AlAs, AlSb, GaN, GaP, GaAs, GaSb, InN, InP, InAs, and InSb, ternary compounds including AlNP, AlNAs, AlNSb, AlPAs, AlPSb, GaNP, GaNAs, GaNSb, GaPAs, GaPSb, InNP, InNAs, InNSb, InPAs, and InPSb, and quaternary compounds include GaAlNAs, GaAlNSb, GaAlPAs, GaInNP, GaInNAs, GaInNSb, GaInPAs, GaInPSb, InAlNP, InAlNAs, InAlNSb, InAlPAs, and InAlPSb; Group IV-VI f semiconductor compounds, including: binary compounds including SnS, SnSe, SnTe, PbSe, PbS, and PbTe, ternary compounds including SnSeS, SnSeTe, SnSTe, SnPbS, SnPbSe, SnPbTe, PbSTe, PbSeS, and PbSeTe, and quaternary compounds including SnPbSSe, SnPbSeTe, and SnPbSTe.

In some embodiments, the light-emitting quantum dot comprises a Group II-VI semiconductor compound. In some preferred embodiments, the light-emitting quantum dot is selected from the group consisting of CdSe, CdS, CdTe, ZnO, ZnSe, ZnS, ZnTe, HgS, HgSe, HgTe, CdZnSe, and any combination thereof. In a suitable embodiment, this material is used as light-emitting quantum dots for visible light due to the relatively well-established synthesis scheme of CdSe and CdS.

In another preferred embodiment, the light-emitting quantum dots comprise a Group III-V semiconductor compound, preferably selected from the group consisting of InAs, InP, InN, GaN, InSb, InAsP, InGaAs, GaAs, GaP, GaSb, AlP, AlN, AlAs, AlSb, CdSeTe, ZnCdSe, and any combination thereof.

In another preferred embodiment, the light-emitting quantum dots comprise Group IV-VI semiconductor compound, preferably selected from the group consisting of PbSe, PbTe, PbS, PbSnTe, Tl₂SnTe₅, and any combination thereof.

In a preferred embodiment, the quantum dots have a core-shell structure. The core and the shell respectively include one or more identical or different semiconductor materials.

In the quantum dots having a core-shell structure, the shell may comprise a monolayer or multilayer structure. The shell comprises one or more semiconductor materials that are the same as or different from the core. In a preferred embodiment, the shell has a thickness of about 1 to 20 layers. In a more preferred embodiment, the shell has a thickness of about 5 to 10 layers. In certain embodiments, two or more shells grow on the surface of the quantum dot care.

In a preferred embodiment, the semiconductor material used for the shell has a larger band gap than the core. Particularly preferred, the core has a type I semiconductor heterojunction structure.

In another preferred embodiment, the semiconductor material used for the shell has a smaller band gap than the core.

In a preferred embodiment, the semiconductor material used for the shell has the same or similar atomic crystal structure as the core. This choice is conducive to reducing the stress between the core and shell, making the quantum dots more stable.

Examples of suitable light-emitting quantum dots employing core-shell structures are (but not limited to):

Red light: CdSe/CdS, CdSe/CdS/ZnS, CdSe/CdZnS, and the like:

Green light: CdZnSe/CdZnS, CdSe/ZnS, and the like:

Blue light: CdS/CdZnS, CdZnS/ZnS, and the like.

The present disclosure further relates to a formulation or ink.

The formulation or ink comprises the above spirocyclic derivative, the above polymer or the above mixture, and an organic solvent.

The present disclosure further provides a film comprising the above spirocyclic derivative or the above polymer and prepared by a solution.

The viscosity and surface tension of ink are important parameters when the ink is used in the printing process. Appropriate surface tension parameter of the ink is suitable to the specific substrate and the specific printing method.

In a preferred embodiment, the ink has a surface tension at working temperature or at 25° C. in the range of about 19 dyne/cm to about 50 dyne/cm, in a more preferred embodiment, the ink has a surface tension at working temperature or at 25° C. in the range of 22 dyne/cm to 35 dyne/cm, and in a most preferred embodiment, the ink has a surface tension at working temperature or at 25° C. in the range of 25 dyne/cm to 33 dyne/cm.

In another preferred embodiment, the ink has a surface tension at the working temperature or at 25° C. in the range of about 1 cps to 100 cps, in a more preferred embodiment, the ink has a surface tension at the working temperature or at 25° C. in the range of 1 cps to 50 cps, in a still more preferred embodiment, the ink has a surface tension at the working temperature or at 25° C. in the range of 1.5 cps to 20 cps, and in a most preferred embodiment, the ink has a surface tension at the working temperature or at 25° C. in the range of 4.0 cps to 20 cps. The formulation thus formulated will be suitable for inkjet printing.

The viscosity can be adjusted by various methods, such as by selecting the appropriate solvent and the concentration of the function material in the ink. The ink comprising the above spirocyclic derivative, the above polymer or the above mixture can facilitate the adjustment of the printing ink in an appropriate range according to the printing method used.

In general, the above spirocyclic derivative, the polymer or the above mixture in the formulation has a weight ratio in the range of 0.3 wt % to 30 wt %/o in a preferred embodiment, the above spirocyclic derivative, the polymer or the above mixture in the formulation has a weight ratio in the range of 0.5 wt % to 20 wt %, in a more preferred embodiment, the above spirocyclic derivative, the polymer or the above mixture in the formulation has a weight ratio in the range of 0.5 wt % to 15 wt %, in a still more preferred embodiment, the above spirocyclic derivative, the polymer or the above mixture in the formulation has a weight ratio in the range of 0.5 wt % to 10 wt %, and in a most preferred embodiment, the above spirocyclic derivative, the polymer or the above mixture in the formulation has a weight ratio in the range of 1 wt % to 5 wt %.

In some embodiments, the organic solvent is selected from solvents based on aromatics or heteroaromatics, especially aliphatic chain/ring substituted aromatic solvents, or aromatic ketone solvents, or aromatic ether solvents.

In some embodiments, the organic solvent is selected from the solvents based on aromatics or heteroaromatics, such as p-diisopropylbenzene, pentylbenzene, tetrahydronaphthalene, cyclohexyl benzene, chloronaphthalene, 1,4-dimethylnaphthalene, 3-isoprpylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isoprpylbiphenyl, p-cymene, 1-methylnaphthalene, 1,2,4-trichlorobenzene, 1,3-dipropoxybenzene, 4,4-difluorodiphenylinethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlosodiphenylmethane, 4-(3-phenylpropyl)pyridine, benzylbenzoate, 1,1-di(3,4-dimethylphenyl)ethane, 2-isopropylnaphthalene, dibenzylether, and the like; solvents based on ketones: 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-demayone, 2,5-hexanedione, phorone, di-n-amyl ketone, aromatic ether solvents: 3-phenoxytoluene, butoxybenzene, benzylbutylbenzene, p-anisaldehyde dimethyl acetal, tetrahydro-2-phenoxy-2H-pyran, 1,2-dimethoxy 4-(1-propenyl)benzene, 1,4-benzodioxane, 1,3-dipropylbenzene, 2,5-dimethoxytoluene, 4-ethylphenetole, 1,2,4-trimethoxybenzene, 4-(1 propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents: alkyl octoate, alkyl sebacate, alkyl stearate, alkyl benzoate, alkyl phenylacetate, alkyl cinnamate, alkyl oxalate, alkyl maleate, alkyl lactone, alkyl oleate, and the like.

In some preferred embodiments, the organic solvent is selected from aliphatic ketones, such as 2-nonanone, 3-nonanone, 5-nonanone, 2-demayone, 2,5-hexanedione, 2,6,8-trimethyl-4-demayone, phorone, di-n-pentyl ketone, and the like; or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethyl ether alcohol ethyl methyl ether, triethylene glycol butyl methyl ether, tripropylene glycol dimethyl ether, tetraethylene glycol dimethyl ether, and the like.

In some embodiments, the printing ink further includes another organic solvent. The another organic solvent is selected from methanol ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin, indene, and/or mixtures thereof.

In a preferred embodiment, the formulation is a solution.

In another preferred embodiment, the formulation is a suspension.

The present disclosure further relates to the application of the above formulation as the printing ink to make an organic electron device, especially preferably by a printing method or a coating method.

The appropriate printing technology or coating technology includes, but is not limited to inkjet printing, nozzle printing, typography, screen printing, dip coating, spin coating, blade coating, roller printing, twist roller printing, lithography, flexography, rotary printing, spray coating, brush coating or transfer printing, nozzle printing, slot die coating, and the like. The first preference is inkjet printing, slot die coating, nozzle printing, and typography. The solution or the suspension liquid may further includes one or more components, such as a surfactant compound, a lubricant, a wetting agent, a dispersant, a hydrophobic agent, a binder, to adjust the viscosity and the film forming property and to improve the adhesion property. The detailed information relevant to the printing technology and requirements of the printing technology to the solution, such as solvent, concentration, and viscosity, may be referred to Handbook of Print Media: Technologies and Production Methods, Helmut Kipphan, ISBN 3-540-67326-1.

Based on the above spirocyclic derivative, the present disclosure further provides use of the above spirocyclic derivative or the above polymer in an organic electronic device.

The organic electronic device includes an organic light-emitting diode (OLED), an organic photovoltaic (OPV), an organic light emitting electrochemical cell (OLEEC), an organic field effect transistor (OFET), an organic light emitting field effector, an organic laser, an organic spin electron device, an organic sensor, and an organic plasmon emitting diode, especially an OLED. In some embodiments, the above spirocyclic derivative is used in a light-emitting layer of the OLED device

The present disclosure further relates to an organic electronic device comprising the above spirocyclic derivative or the above polymer;

In general, such organic electronic device includes at least a cathode, an anode, and a functional layer between the cathode and the anode, wherein the functional layer comprises at least the above spirocyclic derivative or the above polymer:

The organic electronic device includes an organic light-emitting diode (OLED), an organic photovoltaic (OPV), an organic light emitting cell (OLEEC), an organic field effect transistor (OFET), an organic light emitting field effector, an organic laser, an organic spin electron device, an organic sensor, and an organic plasmon emitting diode.

In a more preferred embodiment, the organic electronic device is an electroluminescence device, especially an OLED. The electroluminescence device includes a substrate, an anode, a light-emitting layer, and a cathode. The electroluminescence device may optionally include a hole transport layer.

In certain embodiments, the hole transport layer of the electroluminescence device comprises the above spirocyclic derivative or the above polymer.

In a preferred embodiment, the light-emitting layer of the electroluminescence device comprises the above spirocyclic derivative or the above polymer.

In a more preferred embodiment, the light-emitting layer of the electroluminescence device comprises the above spirocyclic derivative or the above polymer and a light-emitting material. The light-emitting material may be selected from a fluorescent light emitter, a phosphorescent light emitter, a TADF material or a light-emitting quantum dot.

The structure of the electroluminescence device is briefly described below, but it is not limited thereto.

The substrate may be opaque or transparent. The transparent substrate may be used to make the transparent luminescent device, which may be referred to, for example, Bulovic et al., Nature, 1996, 380, page 29 and Gu et al., Appl. Phys. Lett., 1996, 68, page 2606. The substrate may be rigid or elastic. The substrate may be plastic, metal, a semiconductor wafer, or glass. In some embodiments, the substrate has a smooth surface. The substrate without any surface defects is the particular ideal selection. In one preferred embodiment, the substrate is flexible and may be selected from a polymer thin film or a plastic which have the glass transition temperature T_{g} larger than 150° C., preferably larger than 200° C., more preferably larger than 250° C., most preferably larger than 300° C. Suitable examples of the flexible substrate are polyethylene terephthalate (PET) and polyethylene 2,6-naphthalate (PEN).

The anode may include a conductive metal, metallic oxide, or a conductive polymer. The anode can inject holes easily into the hole-injection layer (HIL), the hole-transport layer (HTL), or the light-emitting layer. In a preferred embodiment, the absolute value of the difference between the work function of the anode and the HOMO energy level or the valence band energy level of the emitter in the light-emitting layer or of the p-type semiconductor material of the HIL or HTL or the electron-blocking layer (EBL) is smaller than 0.5 eV, preferably smaller than 0.3 eV, most preferably smaller than 0.2 eV. Examples of the anode material include, but are not limited to Al, Cu, An, Ag, Mg, Fe, Co, Ni, Mn, Pd, Pt, ITO, aluminum-doped zinc oxide (AZO), and the like. Other suitable anode materials are known and may be easily selected by one of ordinary skilled in the art. The anode material may be deposited by any suitable technologies, such as the suitable physical vapor deposition method which includes a radio frequency magnetron sputtering, a vacuum thermal evaporation, an electron beam, and the like. In some embodiments, the anode is patterned and structured. A patterned ITO conductive substrate may be purchased from market to prepare the device according to the present disclosure.

The cathode may include a conductive metal or metal oxide. The cathode can inject electrons easily into the electron-injection layer (EIL) or the electron-transport layer (ETL), or directly injected into the light-emitting layer. In a preferred embodiment, the absolute value of the difference between the work function of the cathode and the LUMO energy level or the valence band energy level of the emitter in the light-emitting layer or of the n type semiconductor material as the electron-injection layer (EIL) or the electron-transport layer (ETL) or the hole-blocking layer (HBL) is smaller than 0.5 eV preferably smaller than 0.3 eV, most preferably smaller than 0.2 eV. In principle, all materials capable of using as the cathode of the OLED may be used as the cathode material of the device of the present disclosure. Examples of the cathode material include, but are not limited to, Al, Au, Ag, Ca, Ba, Mg, LiF/Al, MgAg alloy, BaF₂/Al, Cu, Fe, Co, Ni, Mn, Pd, Pt, ITO, and the like. The cathode material may be deposited by any suitable technologies, such as the suitable physical vapor deposition method which includes a radio frequency magnetron sputtering, a vacuum thermal evaporation, an electron beam, and the like.

The OLED may further comprise other functional layers such as hole-injection layer (HIL), hole-transport layer (HTL), electron-blocking layer (EBL), electron-injection layer (EIL), electron-transport layer (ETL), and hole-blocking layer (HBL), or a combination thereof. Materials suitable for use in these functional layers are described in detail above.

In an embodiment, the light-emitting layer of the electroluminescence device comprises the organometallic complexes or the above polymer of the present disclosure, and is prepared by a method of solution processing.

In a preferred embodiment, the electroluminescence device has a light emission wavelength between 300 and 1000 nm, in a more preferred embodiment, the electroluminescence device has a light emission wavelength between 350 and 900 nm, and in a still more preferred embodiment, the electroluminescence device has a light emission wavelength between 400 and 800 nm.

The present disclosure further relates to the use of the above organic electronic device in various electronic devices, including, but not limited to display devices, lighting devices, light sources, sensors, and the like.

The present invention will be described below with reference to following preferred embodiments, but is not limited thereto. It should be understood that the scope of the present invention is defined by the appended claims. Those skilled in the art will appreciate that, guided by the concept of the present invention, various modifications can be made to the embodiments of the invention, without departing from the spirit and scope of the invention as claimed.

The method for synthesizing the compound of the present disclosure is exemplified below, but the present disclosure is not limited to the following examples.

### Example 1 Synthesis of Compound (2-3)

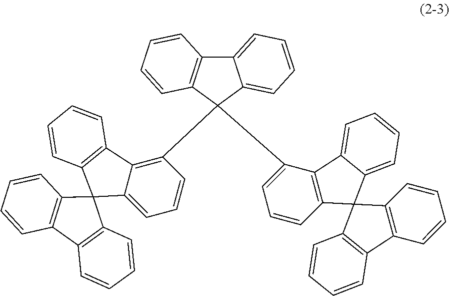 [SMILES: c1ccc2c(c1)-c1ccccc1C21c2ccccc2-c2c1cccc2C1(c2cccc3c2-c2ccccc2C32c3ccccc3-c3ccccc32)c2ccccc2-c2ccccc21]

1)

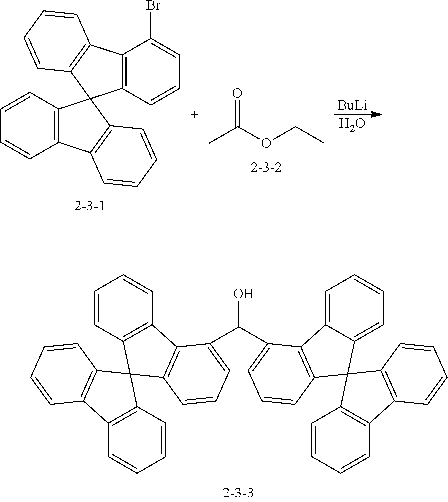

To a 500 ml three-necked flask, compound (2-3-1) (23.7 g, 60 mmol) and 300 ml of anhydrous tetrahydrofuran were added under nitrogen atmosphere. The reaction solution was cooled to −78° C., and n-butyl lithium (60 mmol) was added dropwise slowly. After the completion of the addition, the reaction was continued for 1.5h with the temperature maintained. Ethyl formate (2.64 g, 30 mmol) was added at one shot, and then the reaction was allowed to warm up spontaneously to room temperature, and reacted for 12h, 20 ml of water was added and the reaction solution was stirred and reacted for 0.5h. The reaction stopped and the reaction solution was subject to rotary evaporation to remove most of the solvent, followed by dissolution in dichloromethane, and washed with water for 3 times. The organic solution was collected, mixed with silica gel, purified by column chromatography, with a yield rate of 50%.

2)

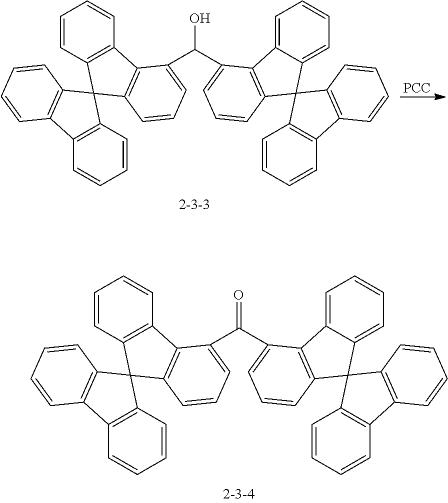

To a 150 ml one-necked flask, compound 2-3-3 (13.2 g, 20 mmol), pyridinium chlorochromate (PCC) (4.3 g, 20 mmol) and 80 ml of dichloromethane were added, stirred for 4h at room temperature. The reaction ended and the organic solution was mixed with silica gel, purified by column chromatography, with a yield rate of 80%.

3)

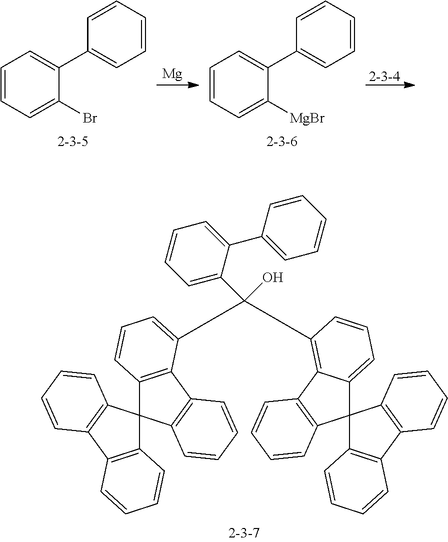

To a 100 ml two-necked flask, Mg (0.72 g, 30 mmol), 5 ml of anhydrous tetrahydrofuran, compound 2-3-5 (1.17 g, 5 mmol) and one grain of iodine were added and heated to initiate Grignard reaction. Compound 2-3-5 (3.5 g, 15 mmol) in 25 ml anhydrous tetrahydrofuran solution was added dropwise slowly after the initiation, stirred and reacted at room temperature for 1h. The reaction solution was heated to resolve the Grignard reagent precipitate produced in the reaction flask, introduced into a 150 ml three-necked flask filled with compound 2-3-4 (6.6 g, 10 mmol) and 40 ml of anhydrous THF, heated to 60° C., and reacted for 12h, 20 ml of water was added and the reaction was continued for 0.5h. The reaction was stopped, and the reaction solution was subjected to rotary evaporation to remove most of the solvent, followed by dissolution in dichloromethane, and washed with water for 3 times. The organic solution was collected, and directly used as a reaction raw material for the next step without further purification after concentration.

4)

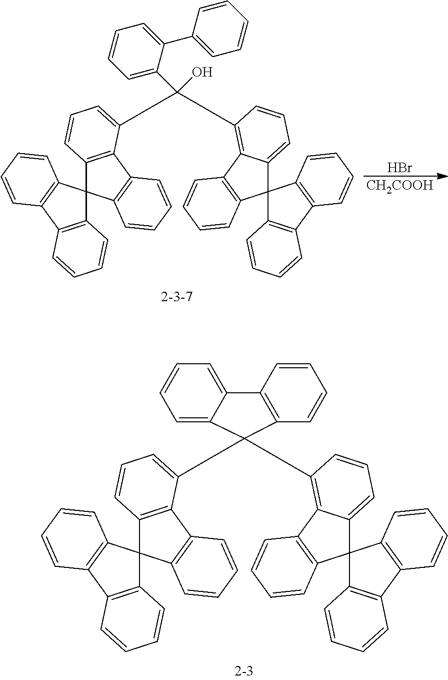

To a 100 ml two-necked flask, compound 2-3-7 described in above step, 30 ml of acetic acid and 15 ml of hydrobromic acid were added, heated to 100° C. stirred and reacted for 12h. The reaction ended. The reaction solution was added into 300 ml of water and suction-filtered, and the residue was recrystallized from a mixed solution of dichloromethane/ethanol, with a yield rate of 80%.

### Example 2 Synthesis of Compound (3-1)

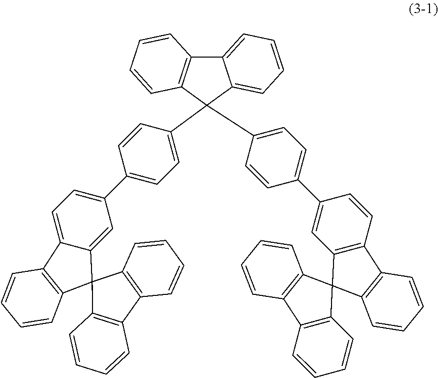 [SMILES: c1ccc2c(c1)-c1ccccc1C2(c1ccc(-c2ccc3c(c2)C2(c4ccccc4-c4ccccc42)c2ccccc2-3)cc1)c1ccc(-c2ccc3c(c2)C2(c4ccccc4-c4ccccc42)c2ccccc2-3)cc1]

1)

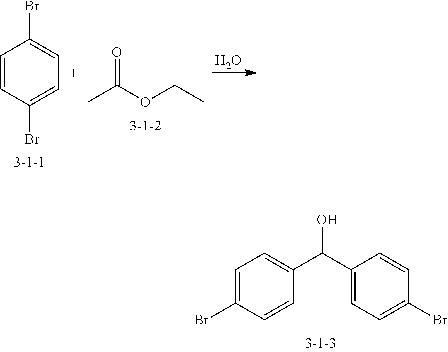

To a 500 ml three-necked flask, 1,4-dibromobenzene (14.2 g, 60 mmol) and 150 ml of anhydrous tetrahydrofuran were added under nitrogen atmosphere, and cooled to −78° C., and n-butyl lithium (60 mmol) was added dropwise slowly. After the completion of the addition, the reaction was continued for 1.5h with the temperature maintained. Ethyl formate (2.64 g, 30 mmol) was added at one shot, and then the reaction was allowed to warm up spontaneously to room temperature, and reacted for 12h. 20 ml of water was added and the reaction mixture was stirred and reacted for 0.5h. The reaction stopped and the reaction solution was subject to rotary evaporation to remove most of the solvent, followed by dissolution in dichloromethane, and washed with water for 3 times. The organic solution was collected, mixed with silica gel, purified by column chromatography, with a yield rate of 60%.

2)

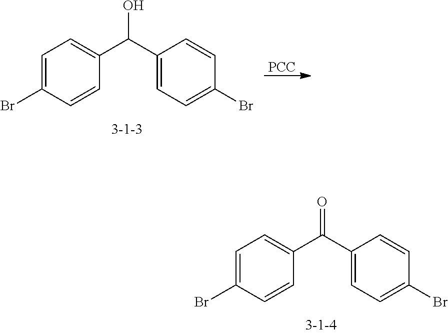

To a 150 ml one-necked flask, compound 3-1-3 (10.3 g, 30 mmol), pyridinium chlorochromate (PCC) (6.5 g, 30 mmol) and 60 ml of dichloromethane were added, and stirred for 4h at room temperature. The reaction ended and the organic solution was mixed with silica gel, purified by column chromatography, with a yield rate of 80%.

3)

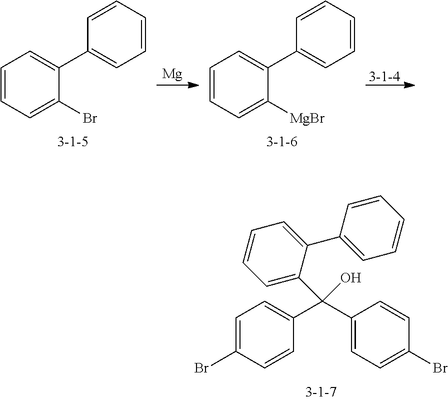

To a 100 ml two-necked flask, Mg (0.72 g, 30 mmol), 5 ml of anhydrous tetrahydrofuran, compound 3-1-5 (1.17 g, 5 mmol) and one grain of iodine were added and heated to initiate the Grignard reaction. Compound 3-1-5 (3.5 g, 15 mmol) in 25 ml anhydrous tetrahydrofuran solution was added dropwise slowly after the initiation, stirred and reacted at room temperature for 1h. The reaction solution was heated to resolve the Grignard reagent precipitate produced in reaction flask, introduced into a 100 ml three-necked flask filled with compound 3-1-4 (3.4 g, 10 mmol) and 20 ml of anhydrous THF, heated to 60° C. and reacted for 12h. 20 ml of water was added and the reaction was continued for 0.5h. The reaction stopped and the reaction solution was subjected to rotary evaporation to remove most of the solvent, followed by dissolution in dichloromethane, and washed with water for 3 times. The organic solution was collected, and directly used as a reaction raw material for the next step without further purification after concentration.

4)

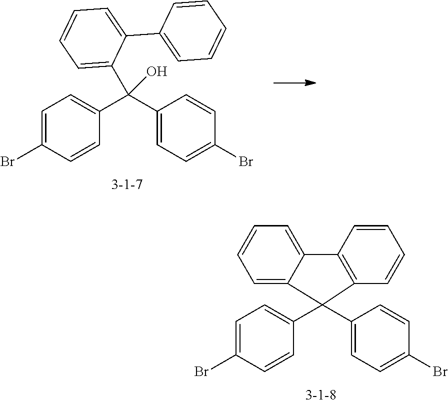 [SMILES: OC(c1ccc(Br)cc1)(c1ccc(Br)cc1)c1ccccc1-c1ccccc1 ; Brc1ccc(C2(c3ccc(Br)cc3)c3ccccc3-c3ccccc32)cc1]

To a 50 mL two-necked flask, compound 3-1-7 described in above step, 10 ml of acetic acid and 5 ml of hydrobromic acid were added, heated to 100° C., stirred and reacted for 12h. The reaction ended. The reaction solution was added into 300 ml of water and suction-filtered, and the residue was recrystallized from a mixed solution of dichloromethane/ethanol, with a yield rate of 90%.

5)

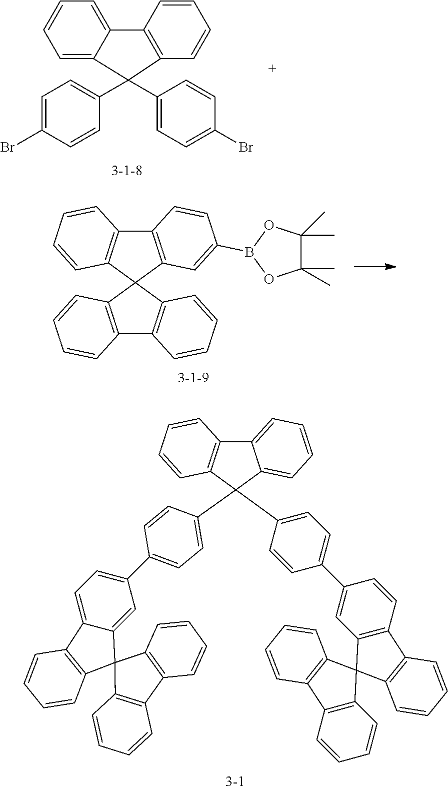

To a 100 ml three-necked flask, compound 3-1-8 (2.38 g, 5 mmol), compound 3-1-9 (4.42 g, 10 mmol), sodium carbonate (2.1 g, 20 mmol), Tetrakis(triphenylphosphine)palladium (0.6 g, 0.5 mmol), 2 ml of water, 30 ml of 1,4-dioxane were added under nitrogen atmosphere, heated to 140° C. and reacted for 12h. The reaction solution was subject to rotary evaporation to remove most of the solvent, followed by dissolution in dichloromethane, and washed with water for 3 times. The organic solution was collected, mixed with silica gel, purified by column chromatography, with a yield rate of 85%.

### Example 3 Energy Structure of the Organic Compound

The energy level of the organic material can be calculated by quantum computation, for example, using TD-DFT (time-dependent density functional theory) by Gaussian03W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-density functional theory) “TD-SCF/DFT/Default Spin/B3PW91” and the basis set “6-31G (d)” (Charge 0/Spin Singlet). The HOMO and LUMO levels are calculated using the following calibration formula, wherein S₁ and T₁ are used directly.

HOMO(eV)=((HOMO(G)×27.212)−0.9899)/1.1206

LUMO(eV)=((LUMO(G)×27.212)−2.0041)/1.385

wherein, HOMO(G) and LUMO(G) are the direct calculation results of Gaussian 03W, in units of Hartree. The results are shown in Table 1:

TABLE 1

| | HOMO | LUMO | T1 | S1 |
|---|---|---|---|---|
| material | [eV] | [eV] | [eV] | [eV] |
| HATCN | −9.04 | −5.08 | 2.32 | 3.17 |
| NPB | −6.72 | −2.85 | 2.97 | 3.46 |
| TCTA | −5.34 | −2.20 | 2.73 | 3.42 |
| 2-3 | −5.95 | −2.24 | 2.86 | 3.96 |
| 3-1 | −5.82 | −2.31 | 2.74 | 3.47 |
| Ir(ppy)₃ | −5.30 | −2.35 | 2.70 | 2.93 |
| B3PYMPM | −5.33 | −2.20 | 2.72 | 3.28 |

### Example 4 Preparation and Characterization of OLED Devices

In the present example, the compounds (2-3) prepared in example 1, (3-1) prepared in example 2 were used as the host materials respectively, Ir(ppy)₃ as the luminescent material. HATCN as the hole-injection material, NPB and TCTA as the hole-transport material, B3PYMPM as an electron-transport material, to make a electroluminescent device have a device structure of ITO/HATCN/NPB/TCTA/host material: Ir(ppy)₃ (15%)/B3PYMPM/LiF/Al.

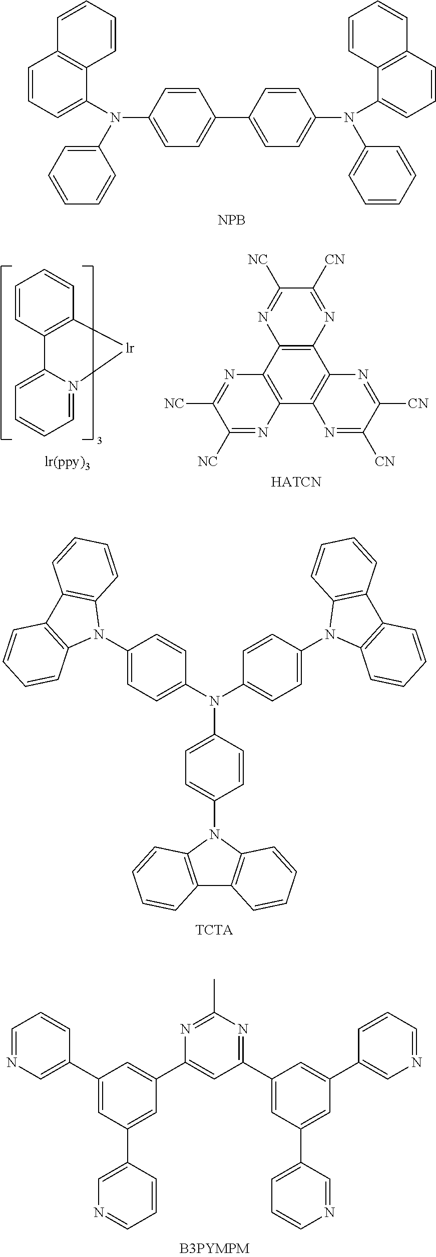

The materials HATCN, NPB, TCTA, B3PYMPM, Ir (ppy)₃ described above are all commercially available, such as from JILIN OLED (Jilin OLED Material Tech Co., Ltd www.jl-oled.com), or the synthesis method thereof are all known, which can be found in the references of the art and will not be described here.

The preparation process of the OLED device above will be described in detail with reference to specific examples below. The structure of the OLED device (as shown in Table 2) is as follows: ITO/HATCN/NPB/TCTA/host material: Ir(ppy)₃/B3PYMPM/LiF/Al. The preparation steps are as follows:

a. cleaning of ITO (indium tin oxide) conductive glass substrate: washing with the use of various solvents (such as one or more of chloroform, acetone or isopropyl alcohol) and then treating with UV and ozone:

b. thermal evaporation deposition in high vacuum (1×10⁻⁶ mbar) with HATCN (5 nm), NPB (40 nm), TCTA (10 nm), host material: 15% Ir(ppy)₃ (15 nm), B3PYMPM (40 nm), LiF (1 nm), Al (100 nm);

c. package: packaging the device in a nitrogen glove box with UV hardened resin.

TABLE 2

| OLED device | Host material |
|---|---|
| OLED1 | (2-3) |
| OLED2 | (3-1) |
| RefOLED | CBP |

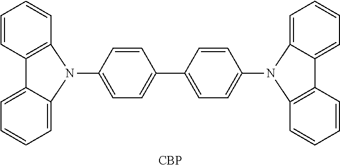 [SMILES: c1ccc2c(c1)c1ccccc1n2-c1ccc(-c2ccc(-n3c4ccccc4c4ccccc43)cc2)cc1 ; "CBP"]

### CBP Purchased from JILIN OLED

The current-voltage (J-V) characteristics of each OLED device are characterized by characterization equipment, while important parameters such as efficiency, lifetime and external quantum efficiency were recorded. It was determined that OLED1 and OLED2 respectively has a luminous efficiency of 2 times or above of that of RefOELD, and a life of 2 times of that of RefOELD. It can be seen that the life of the OLED device prepared by using the organic compound of the present disclosure is greatly improved.

What described above are several embodiments of the present disclosure, and they are specific and in details, but not intended to limit the scope of the present disclosure. It will be understood by those skilled in the art that various modifications and improvements can be made without departing from the concept of the present disclosure, and all these modifications and improvements are within the scope of the present disclosure. The scope of the present disclosure shall be subject to the appended claims.

1-21. (canceled)

22. A spirocyclic derivative having the following general formula (I) 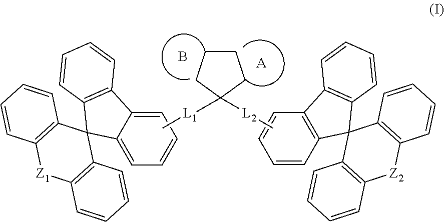
wherein, L₁ or L₂ is a single bond, an aromatic group containing 6 to 40 carbon atoms, or a heteroaromatic group containing 3 to 40 carbon atoms.
A or B is an aromatic group containing 6 to 20 carbon atoms or a heteroaromatic group containing 3 to 20 carbon atoms.
Z₁ or Z₂ is selected from a single bond, N(R), B(R), C(R)₂, Si(R)₂, O, S, C═N(R), C═C(R)₂, P(R), P(═O)R, S═O, or SO₂, or absent,
the hydrogen atoms on L₁, L₂, A, B and the spirocyclic derivative can be substituted by R;
R is an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms, and one or more positions of R may be substituted by H, D, F, CN, alkyl, aralkyl, alkenyl, alkynyl, nitrile group, amino, nitro, acyl, alkoxy, carbonyl, sulfonyl, cycloalkyl, or hydroxy.

23. The spirocyclic derivative according to claim 22, wherein the spirocyclic derivative has a glass transition temperature T_{g} greater than or equal to 100° C.

24. The spirocyclic derivative according to claim 22, wherein the spirocyclic derivative has a triplet level greater than or equal to 2.4 eV.

25. The spirocyclic derivative according to claim 22, wherein L₁ or L₂ is selected from benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, or spirosilabifluorene.

26. The spirocyclic derivative according to claim 22, wherein L₁ or L₂ includes one of the following groups:

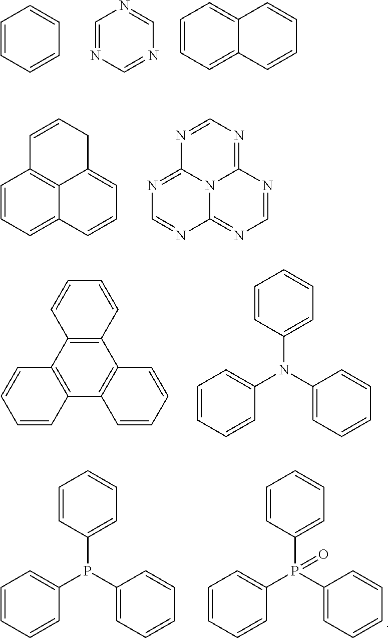

27. The spirocyclic derivative according to claim 22, wherein A or B includes one of the following groups 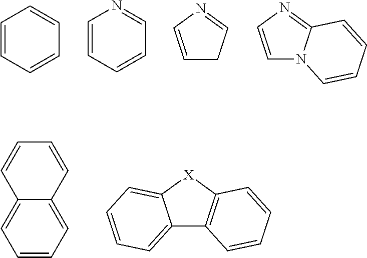
wherein, X is N(R₁), B(R₁), C(R₁)₂, Si(R₁)₂, O, S, C═N(R₁), C═C(R₁)₂, P(R₁), P(═O)R₁, S═O or SO₂;
R₁ is selected from H, D, F, CN, aralkyl, alkenyl, alkynyl, nitrile group, amino, nitro, acyl, alkoxy, carbonyl, sulfonyl, hydroxyl, an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms.

28. The spirocyclic derivative according to claim 27, wherein R₁ is selected from the group consisting of methyl, benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, or spirosilabifluorene.

29. The spirocyclic derivative according to claim 22, wherein the spirocyclic derivative is one selected from compounds having the following structural formula: 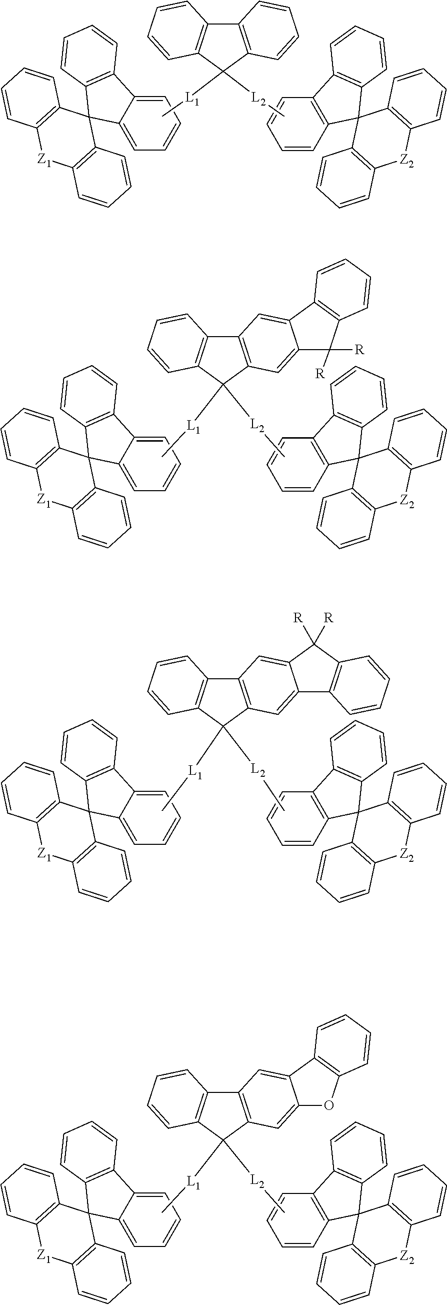 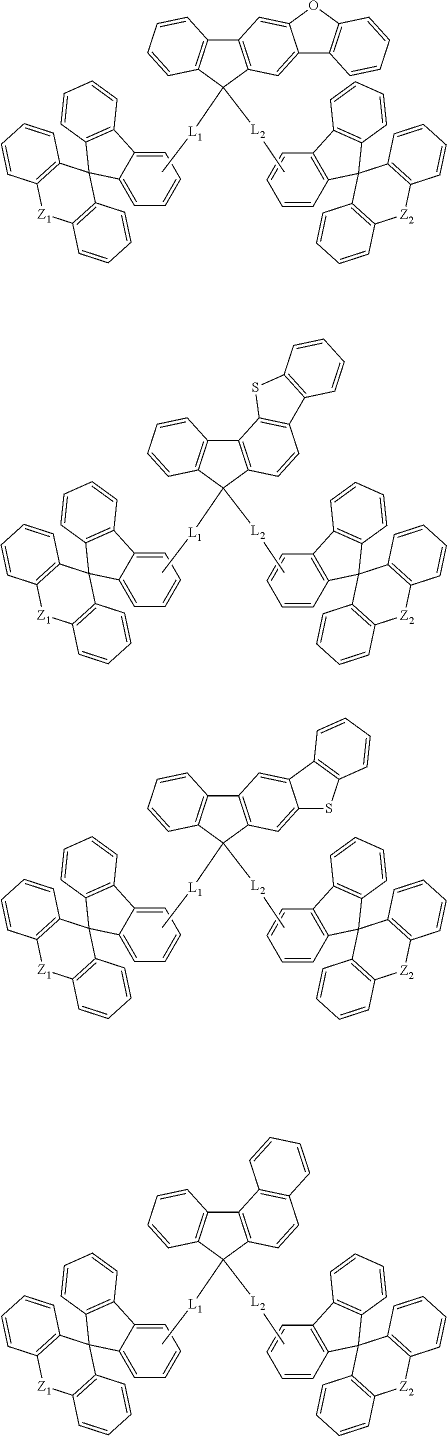 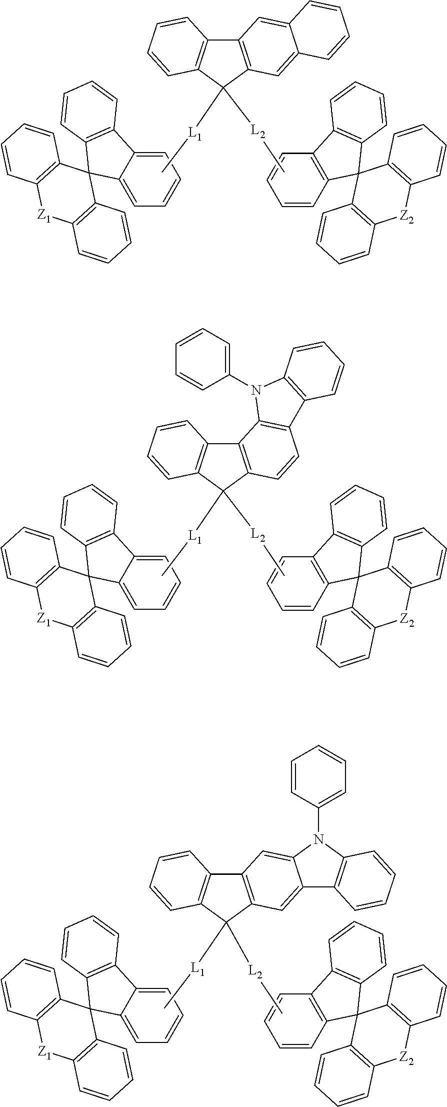 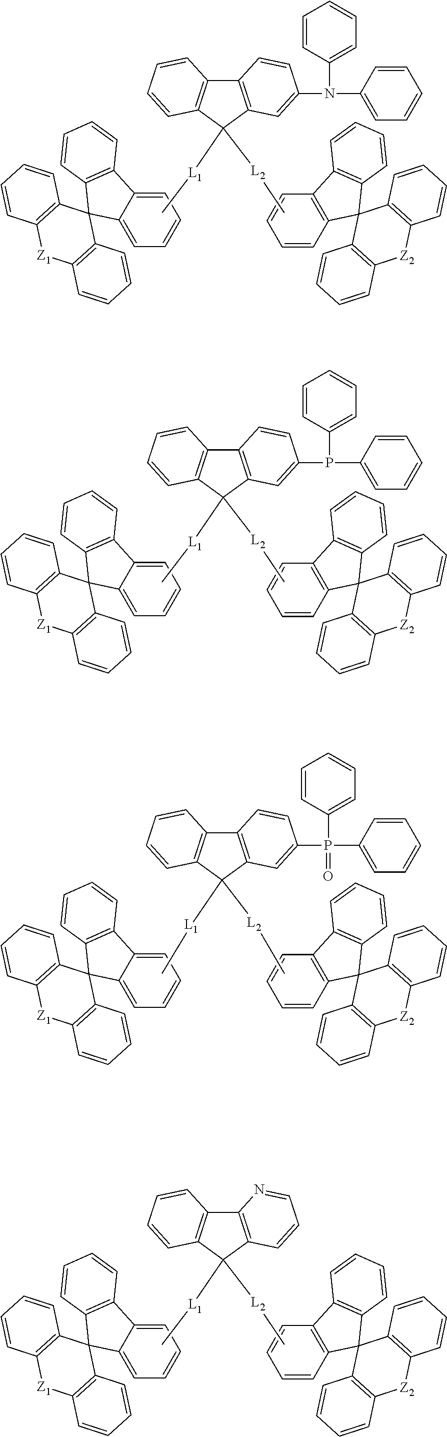 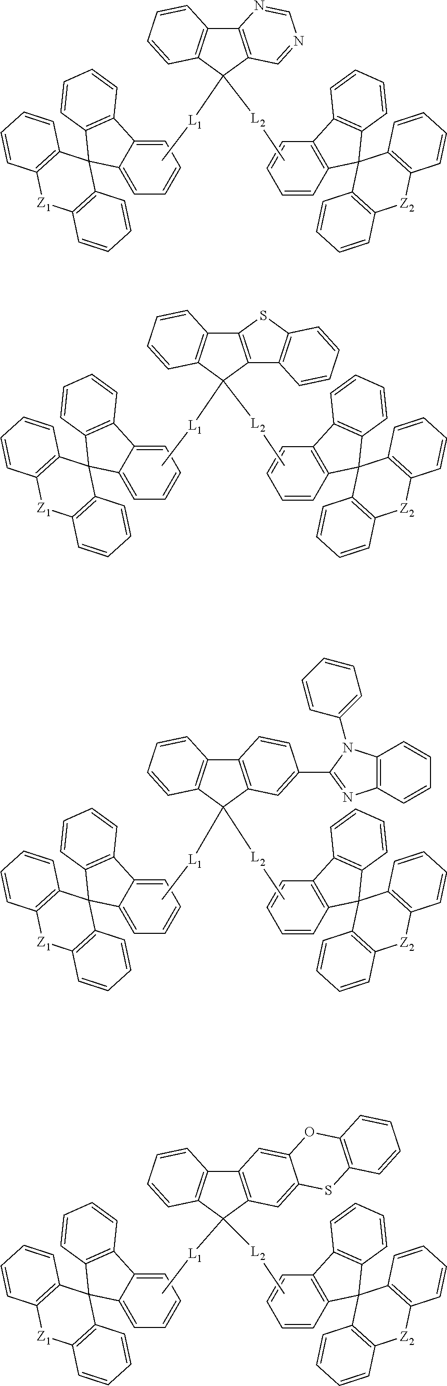 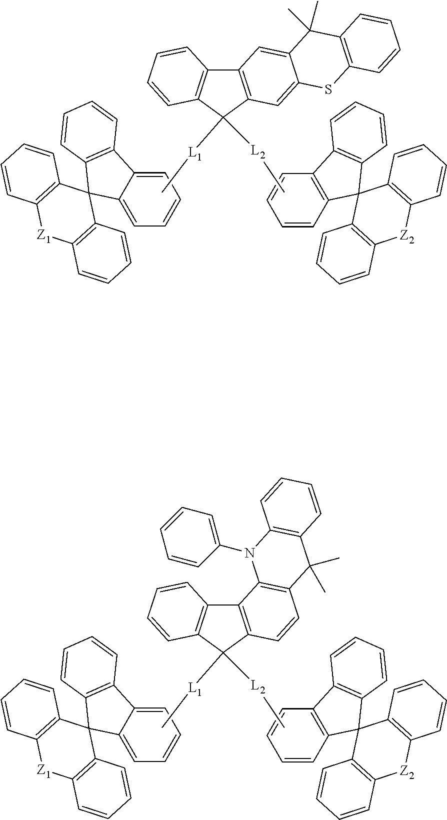
wherein, Z₁, Z₂, L₁, L₂ and R are as defined above.

30. The spirocyclic derivative according to claim 22, wherein the spirocyclic derivative is one selected from compounds having the following structural formula: 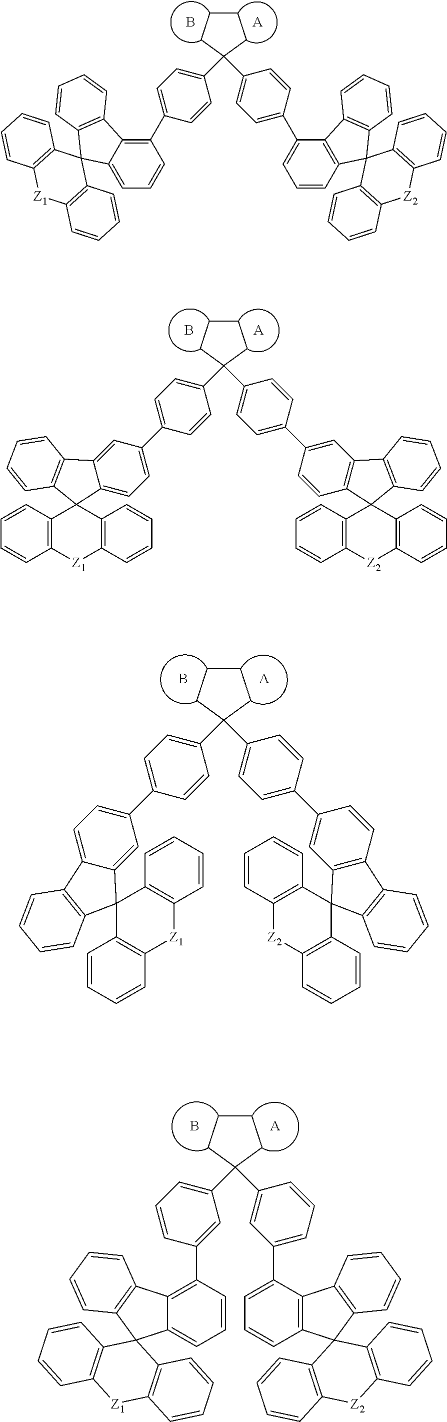 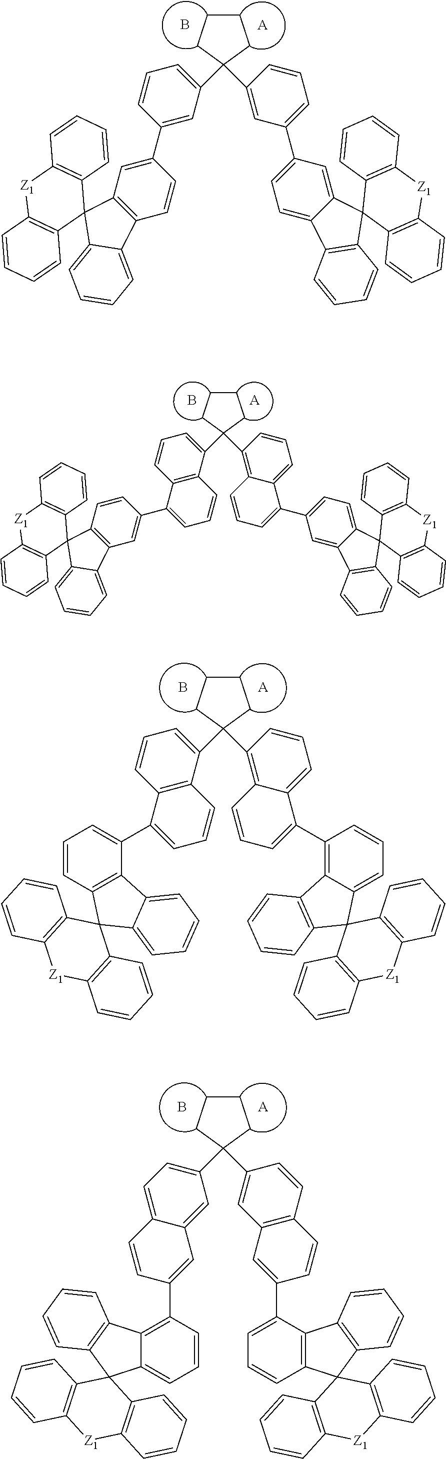 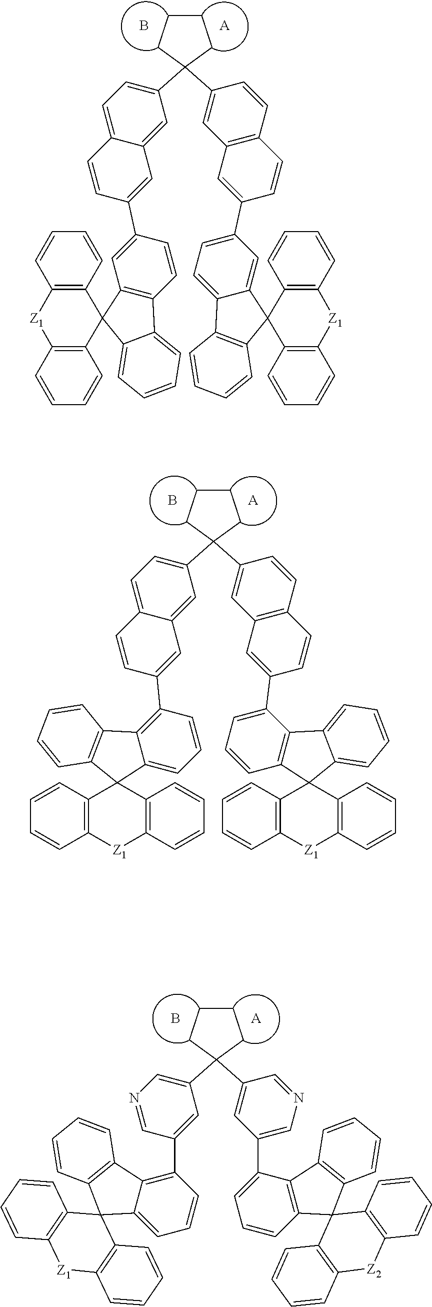 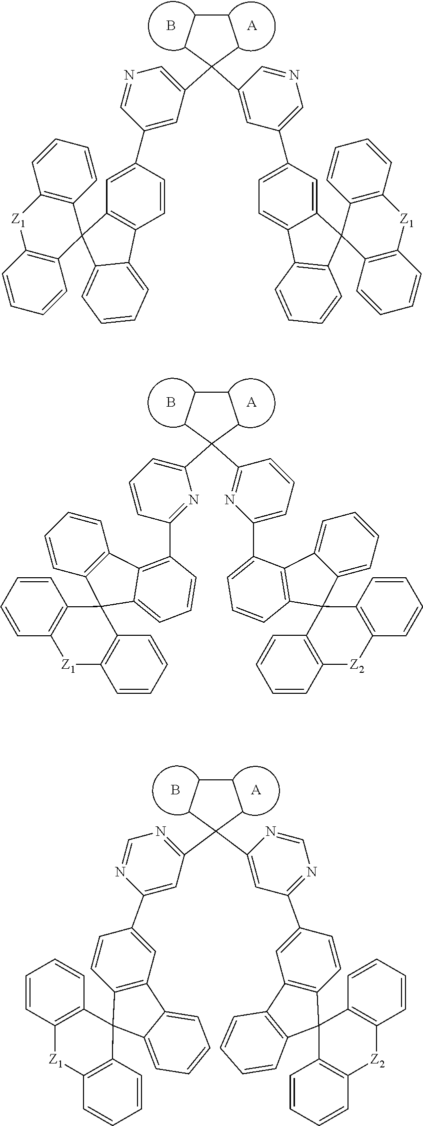 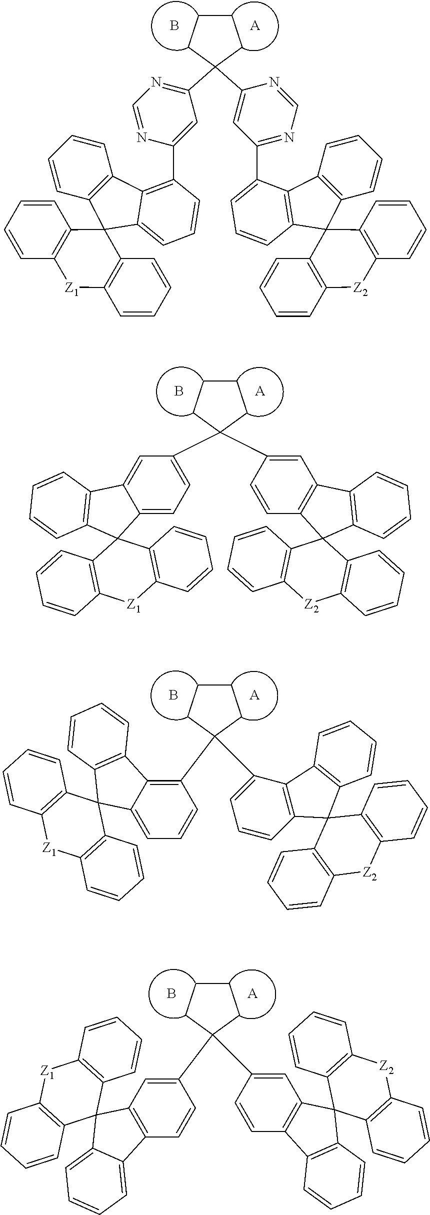 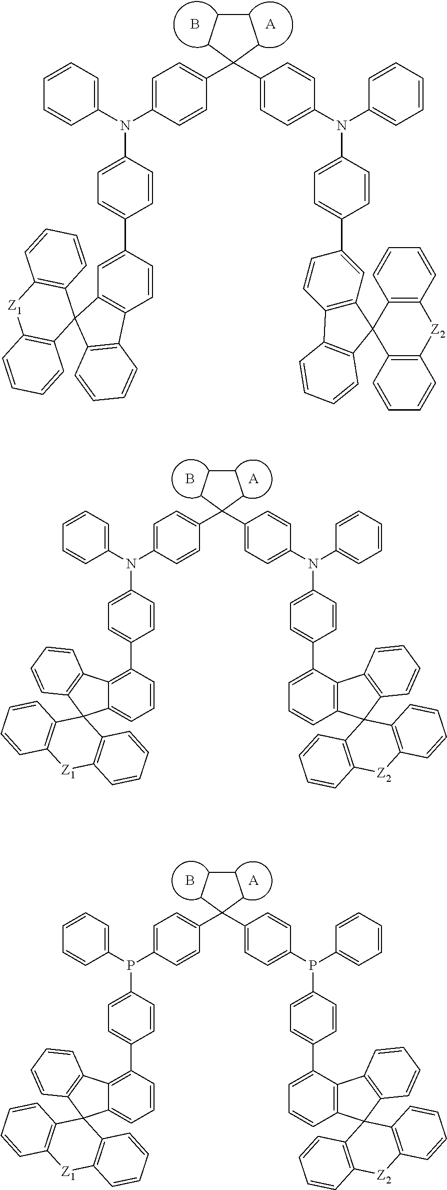 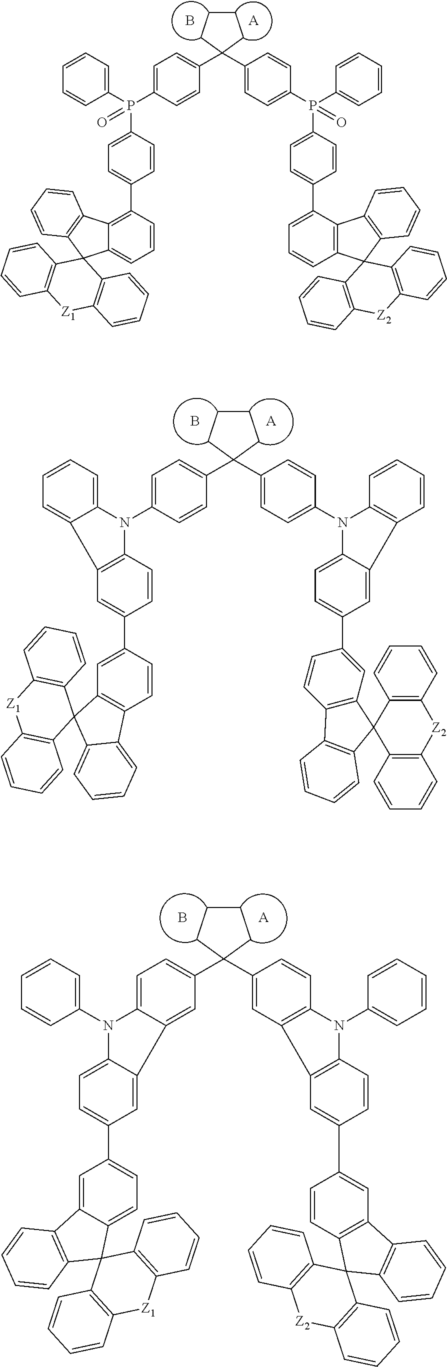
wherein, Z₁, Z₂, A and B are as defined above.

31. The spirocyclic derivative according to claim 22, wherein said spirocyclic derivative is one selected from compounds having the following structural formula:

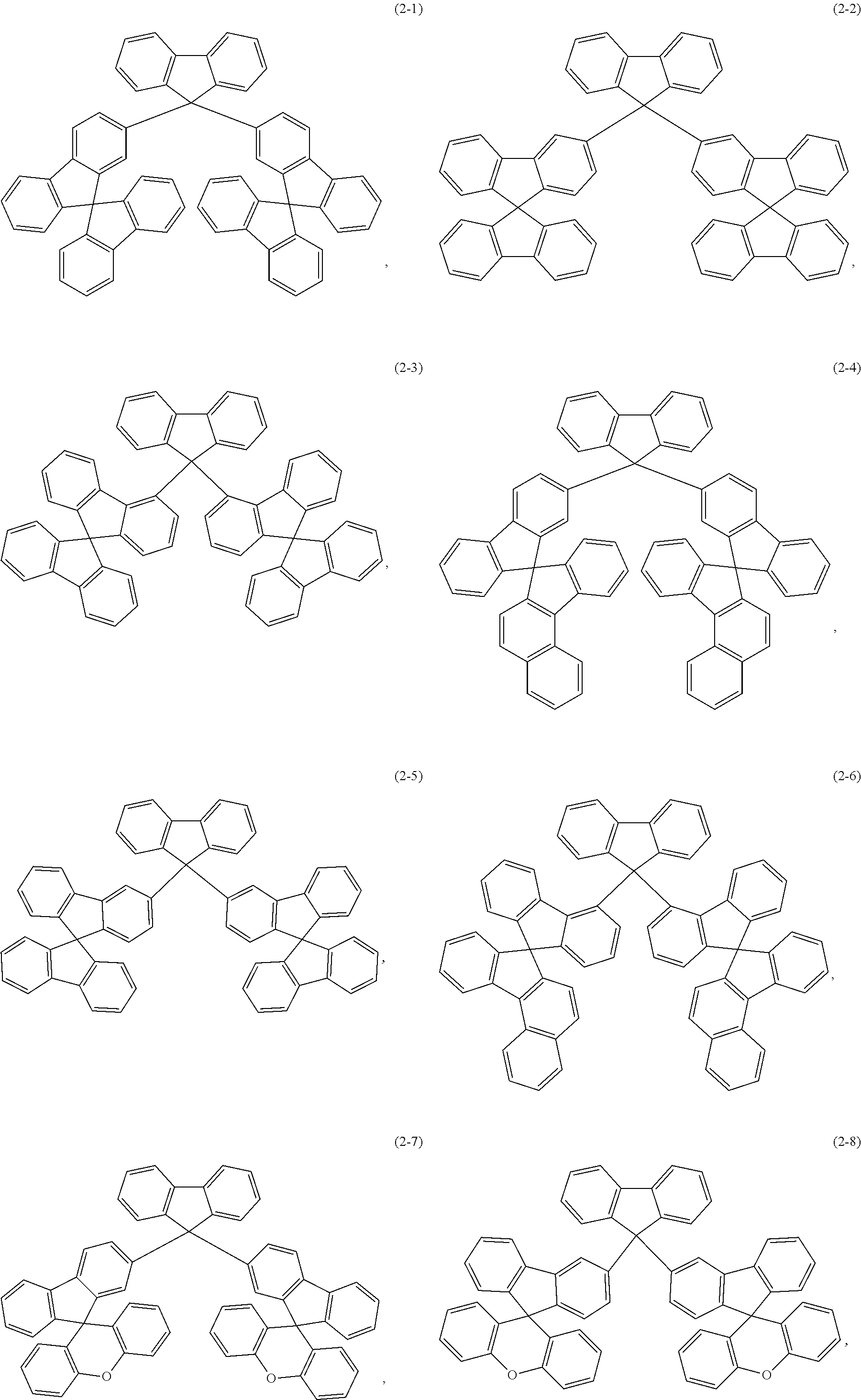

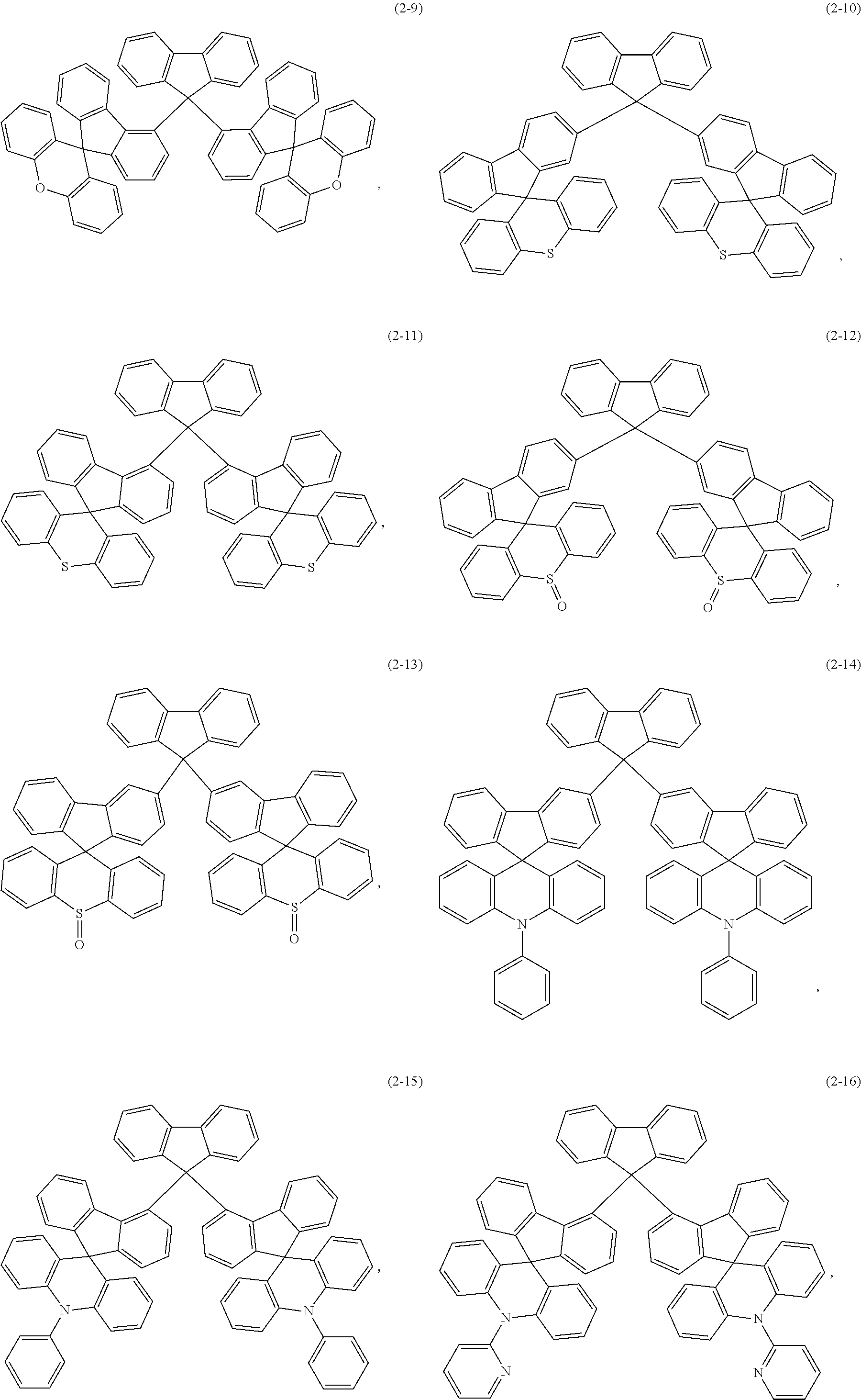

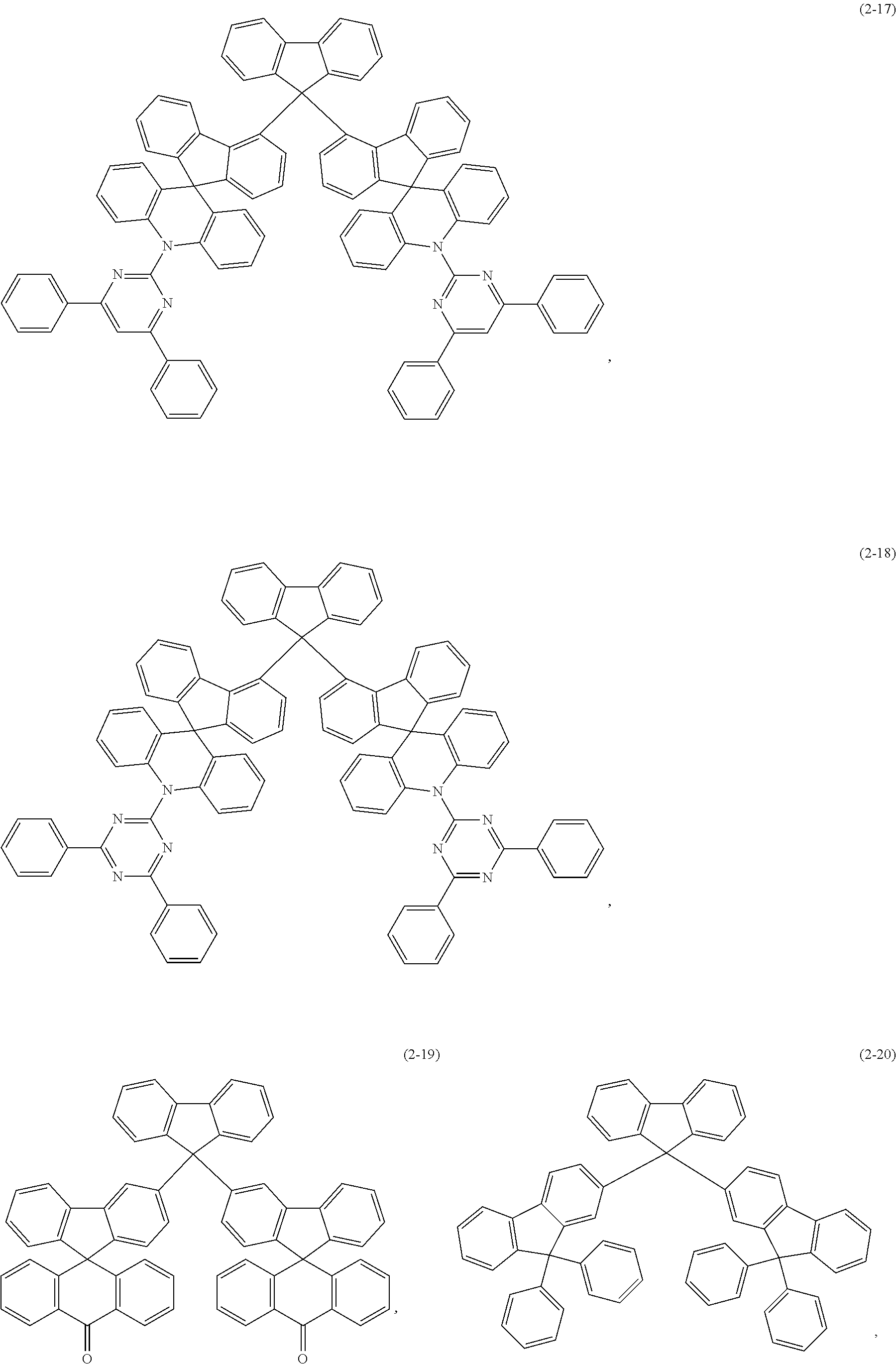

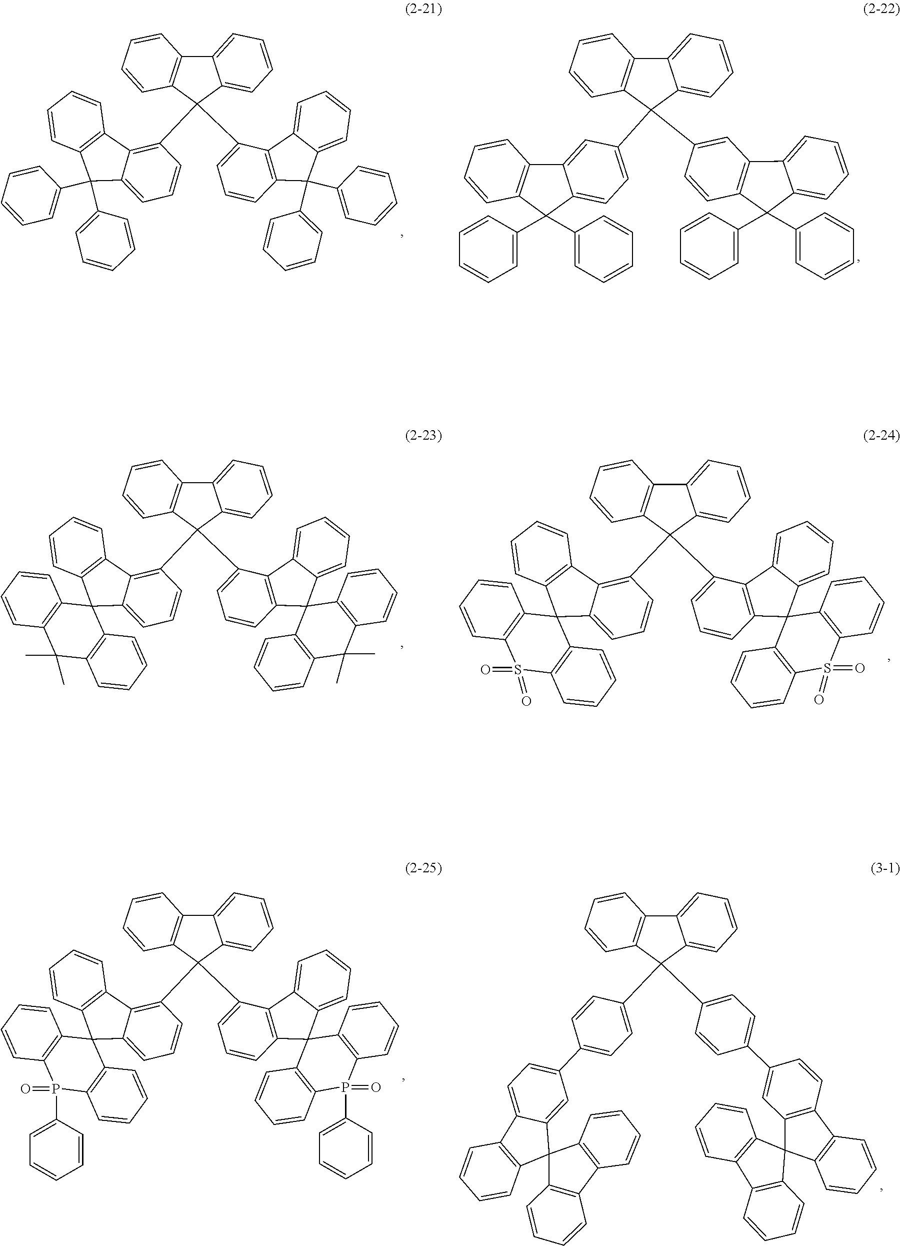

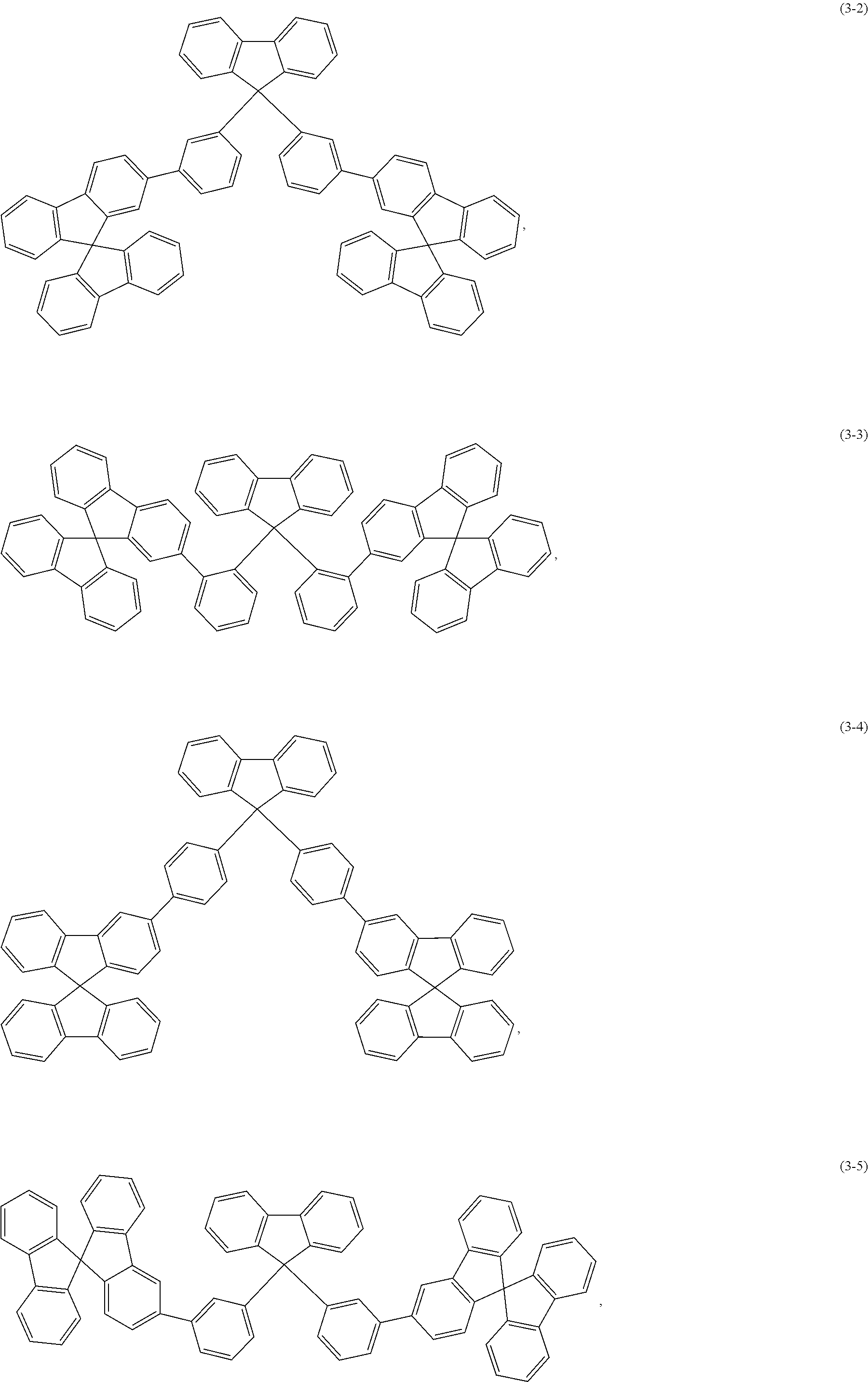

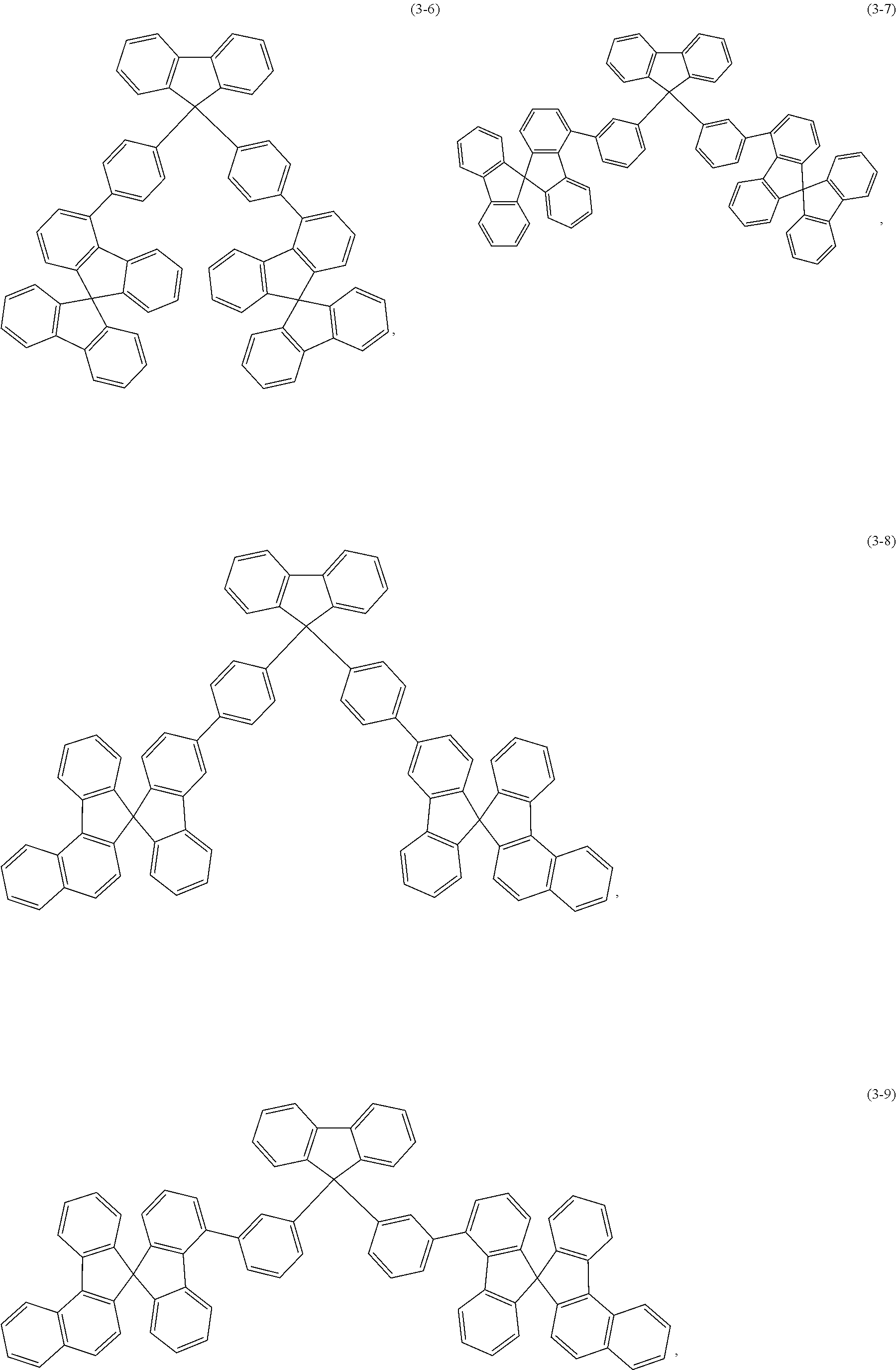

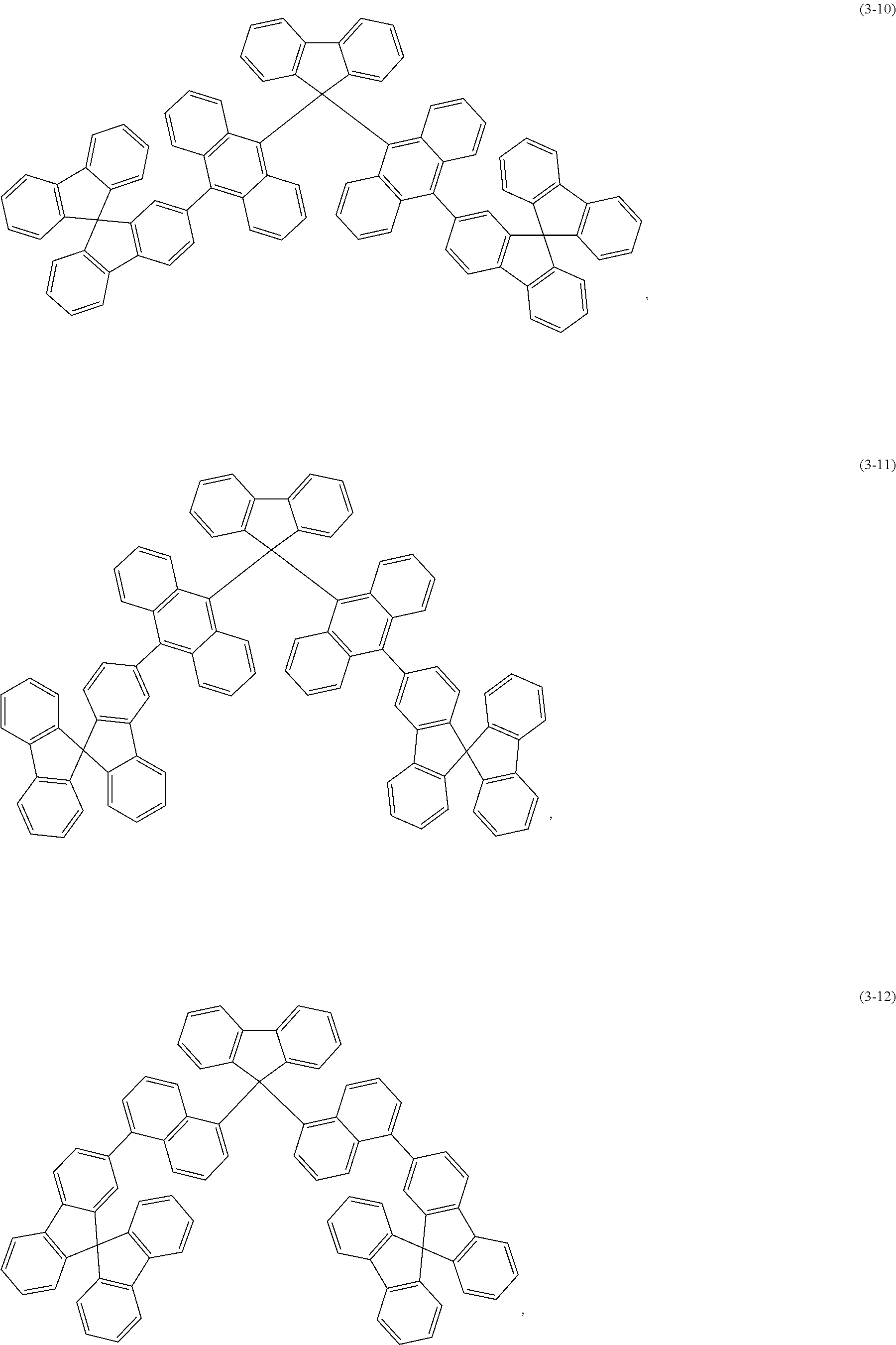

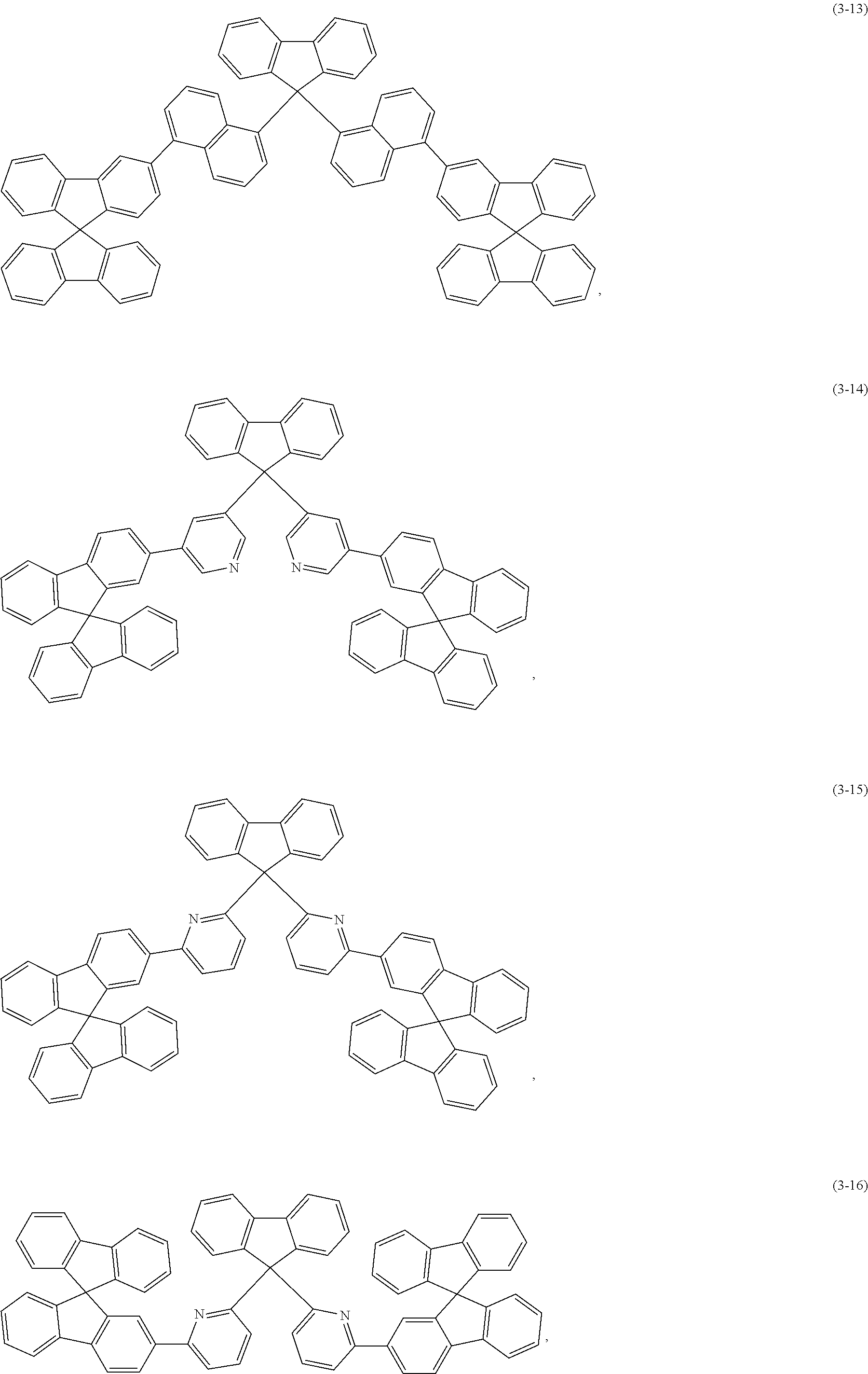

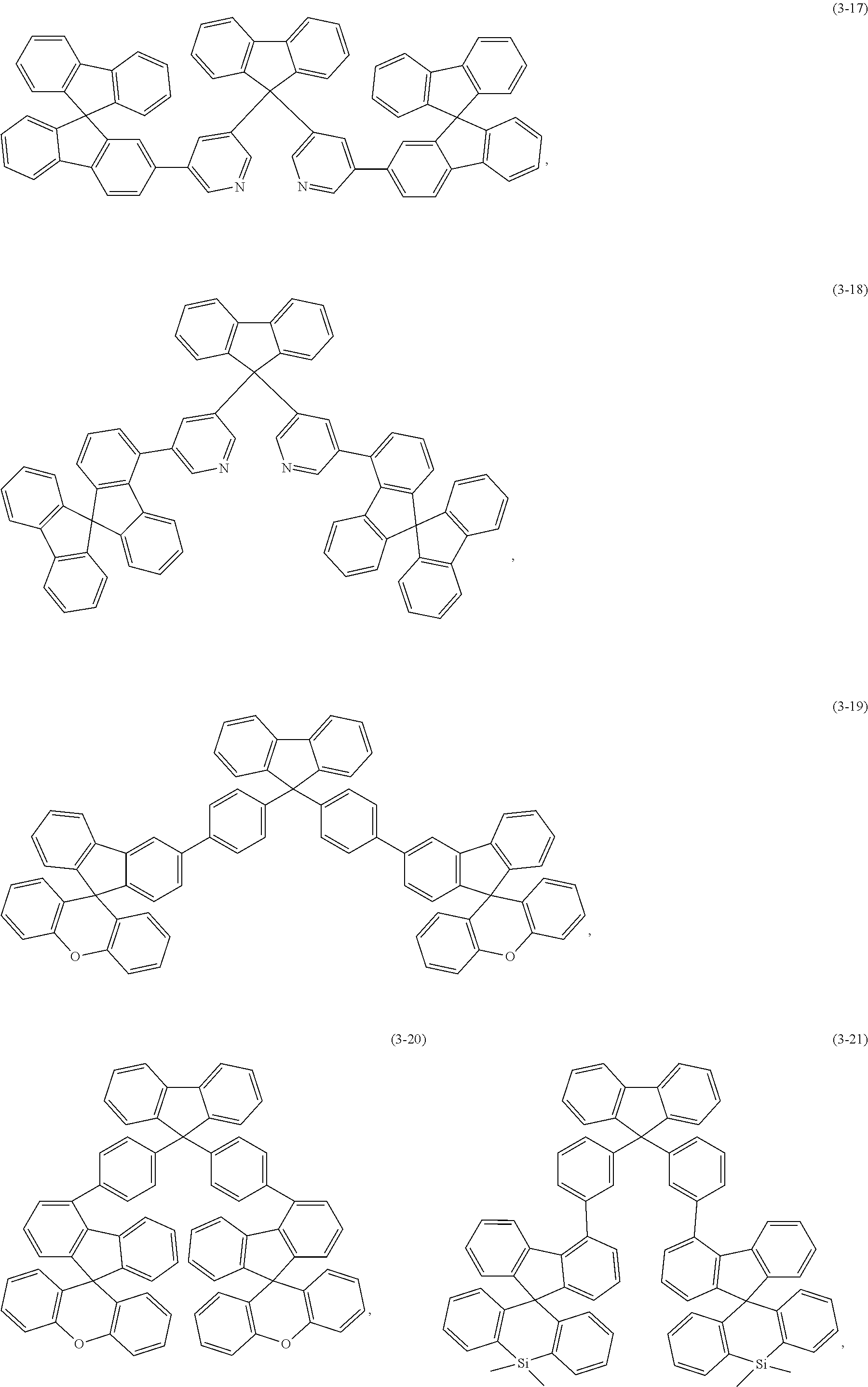

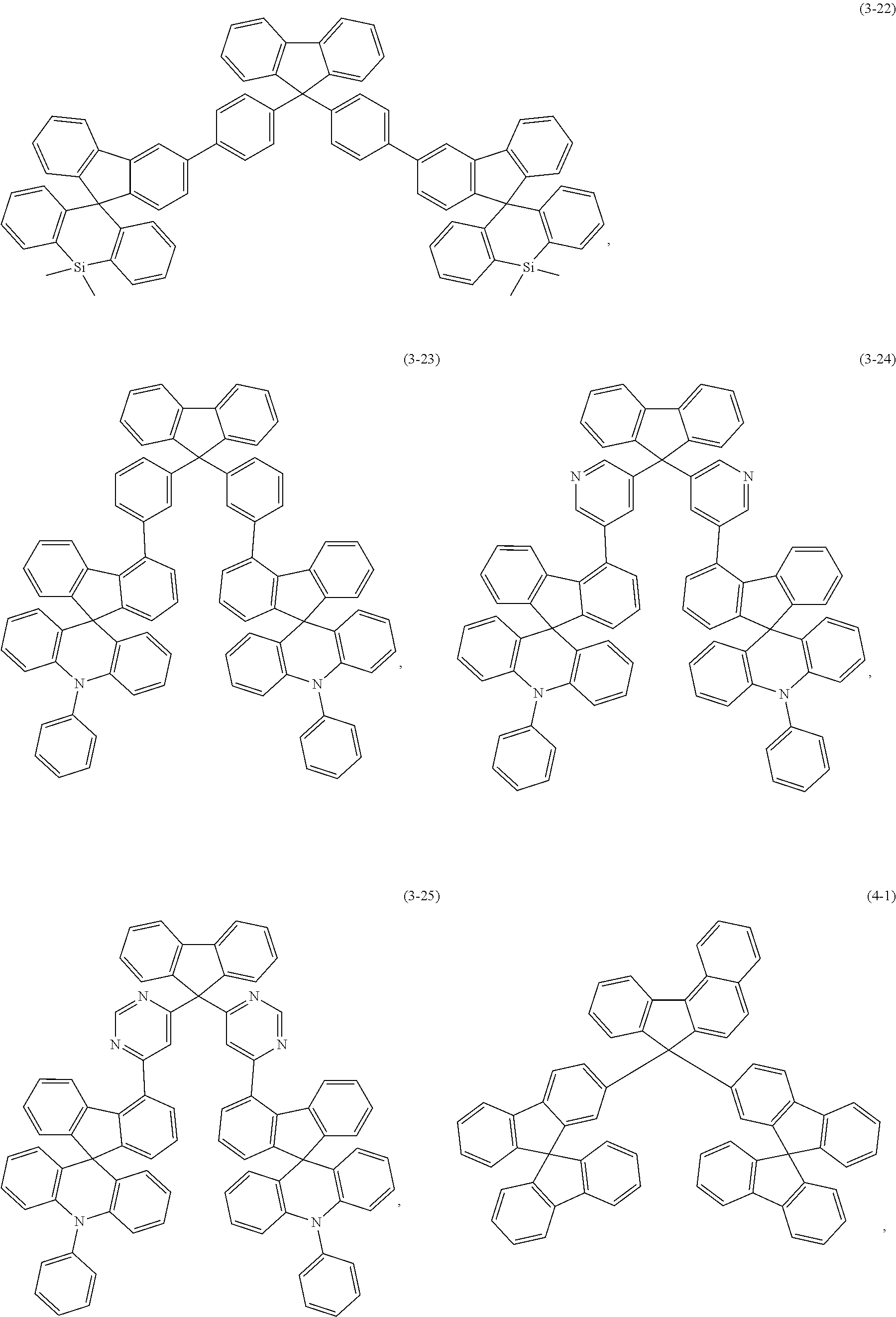

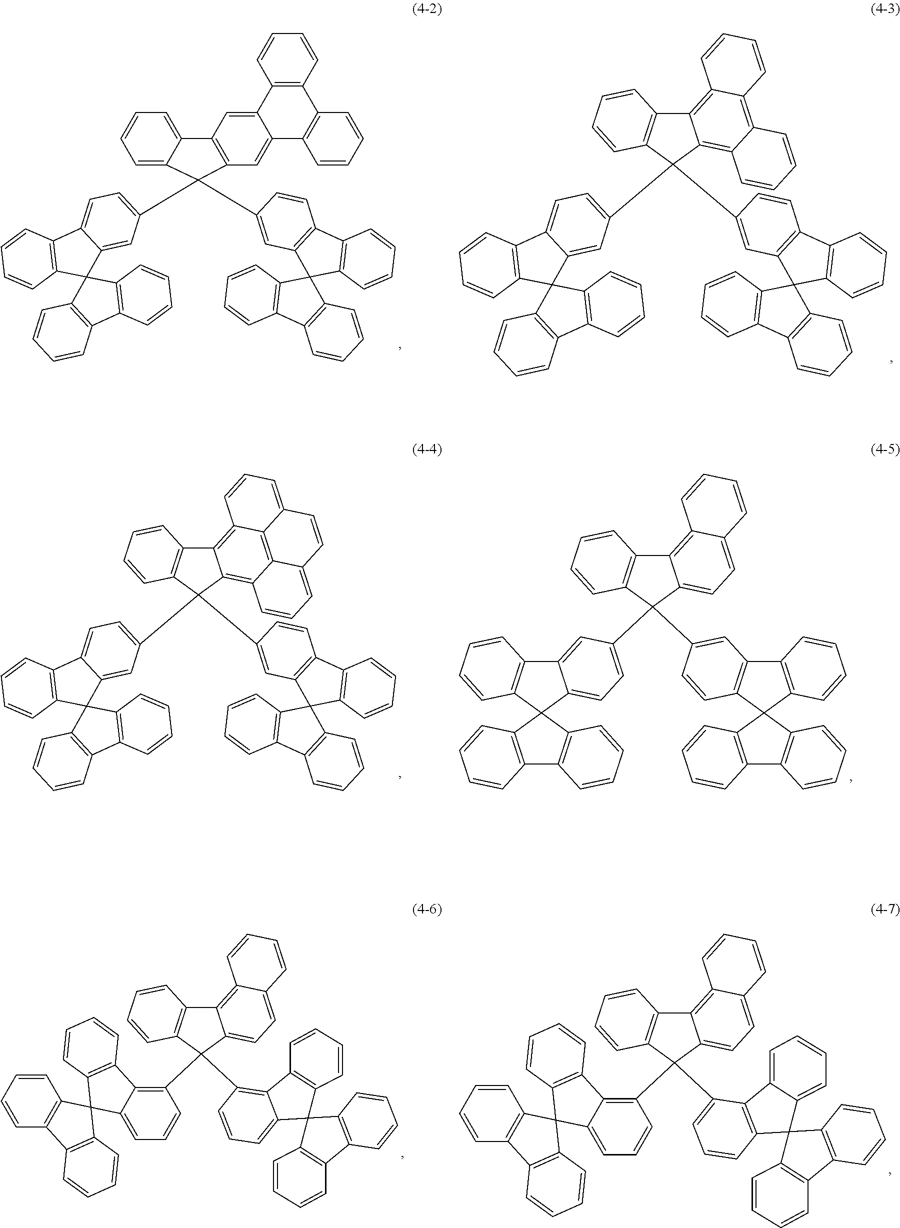

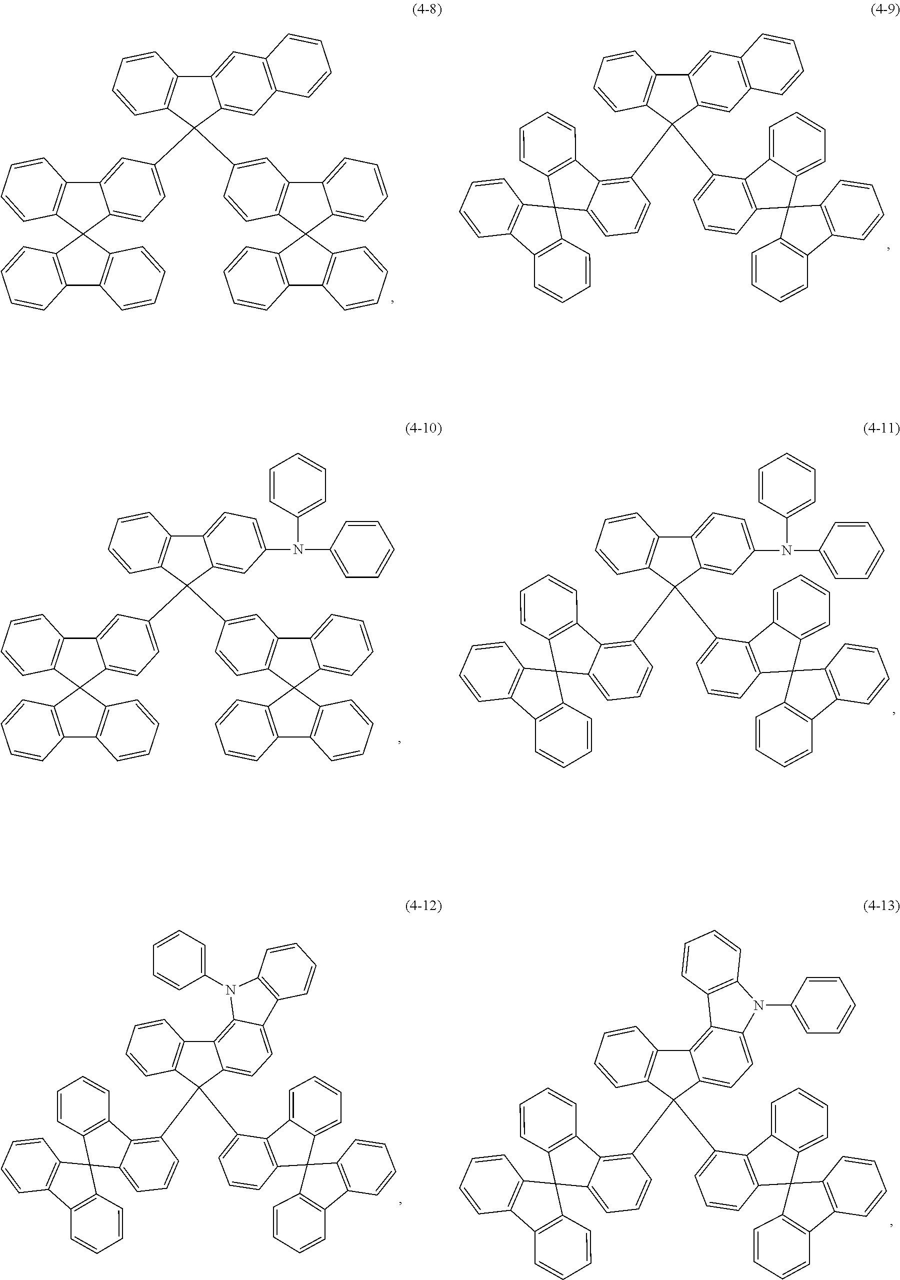

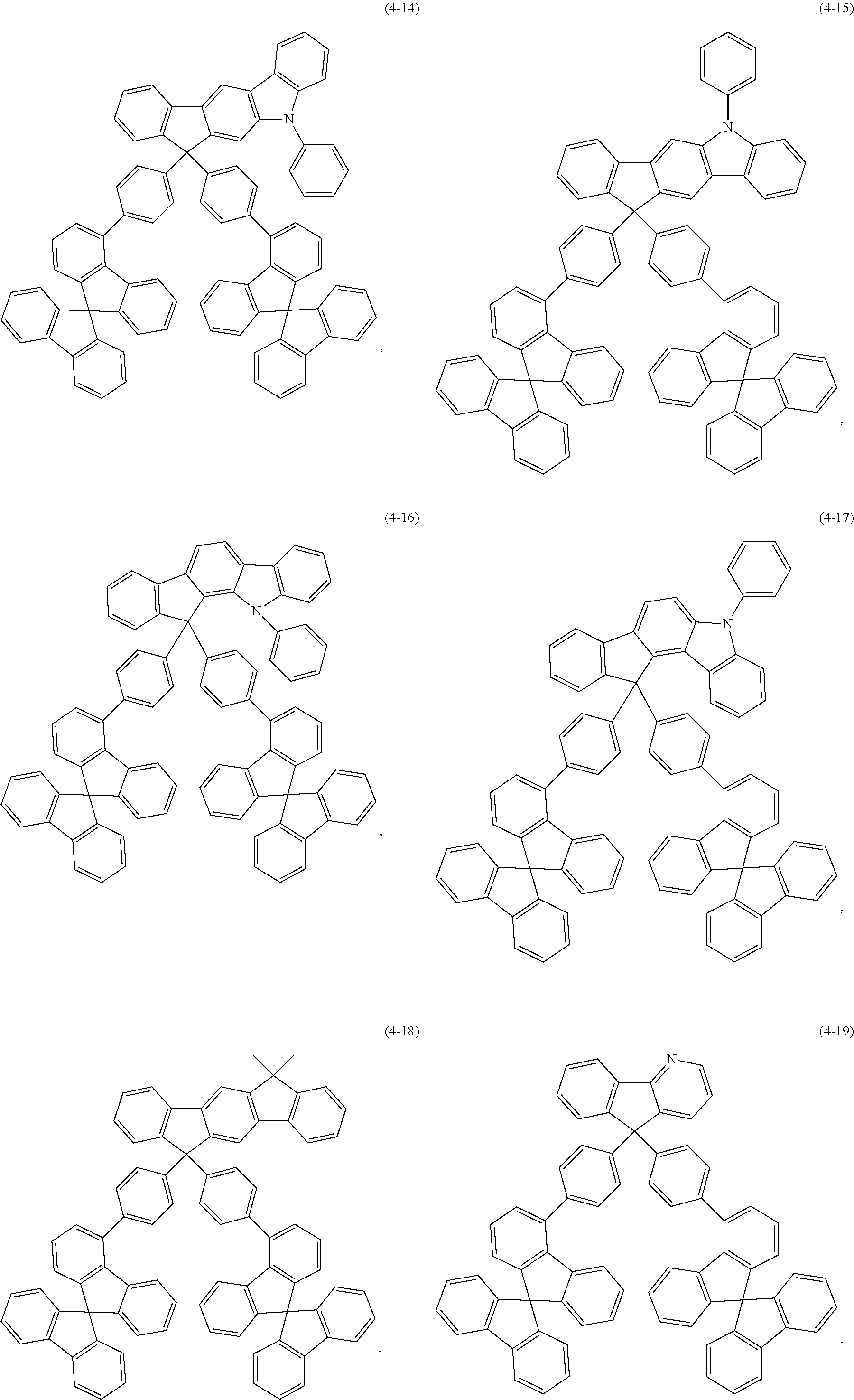

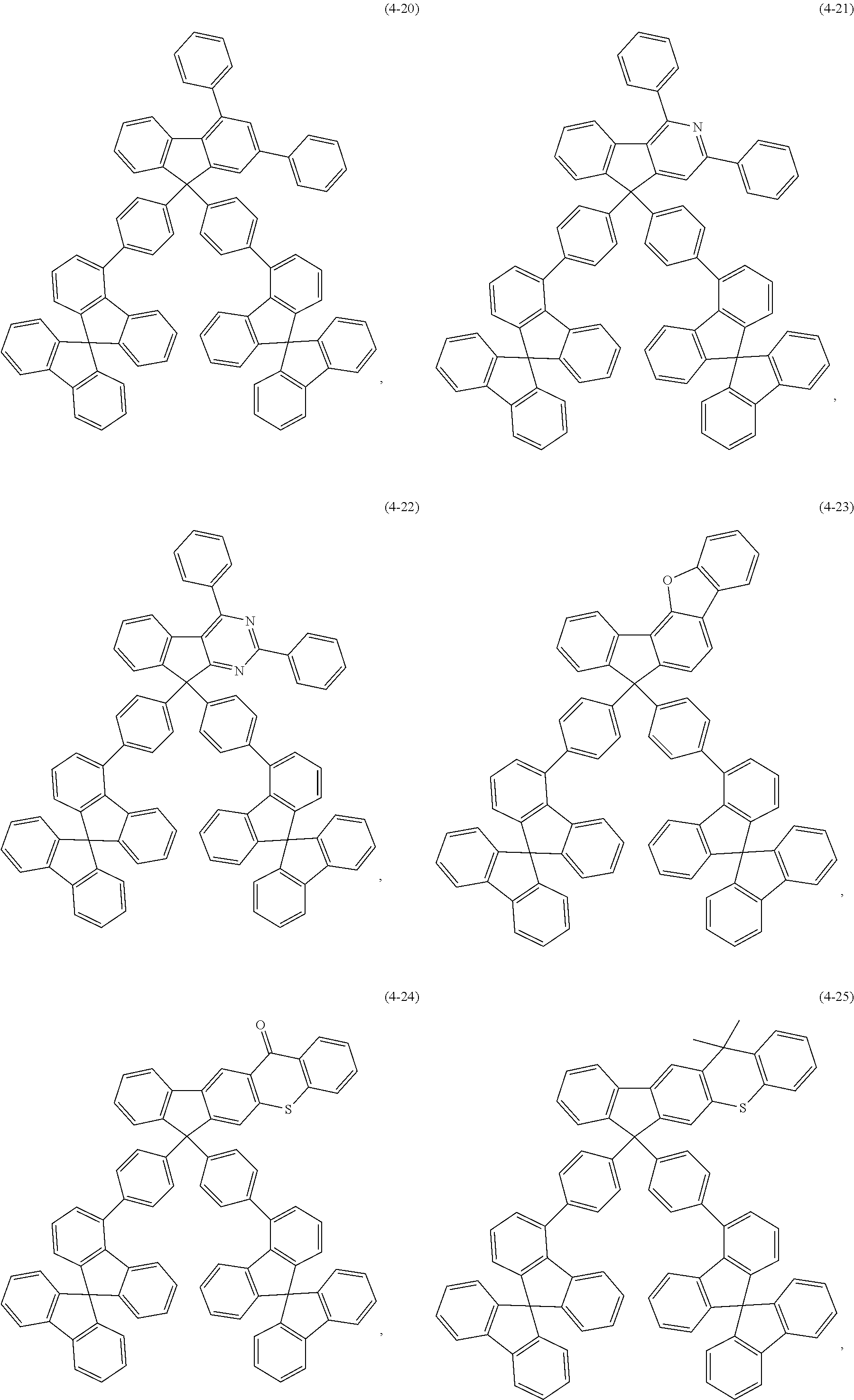

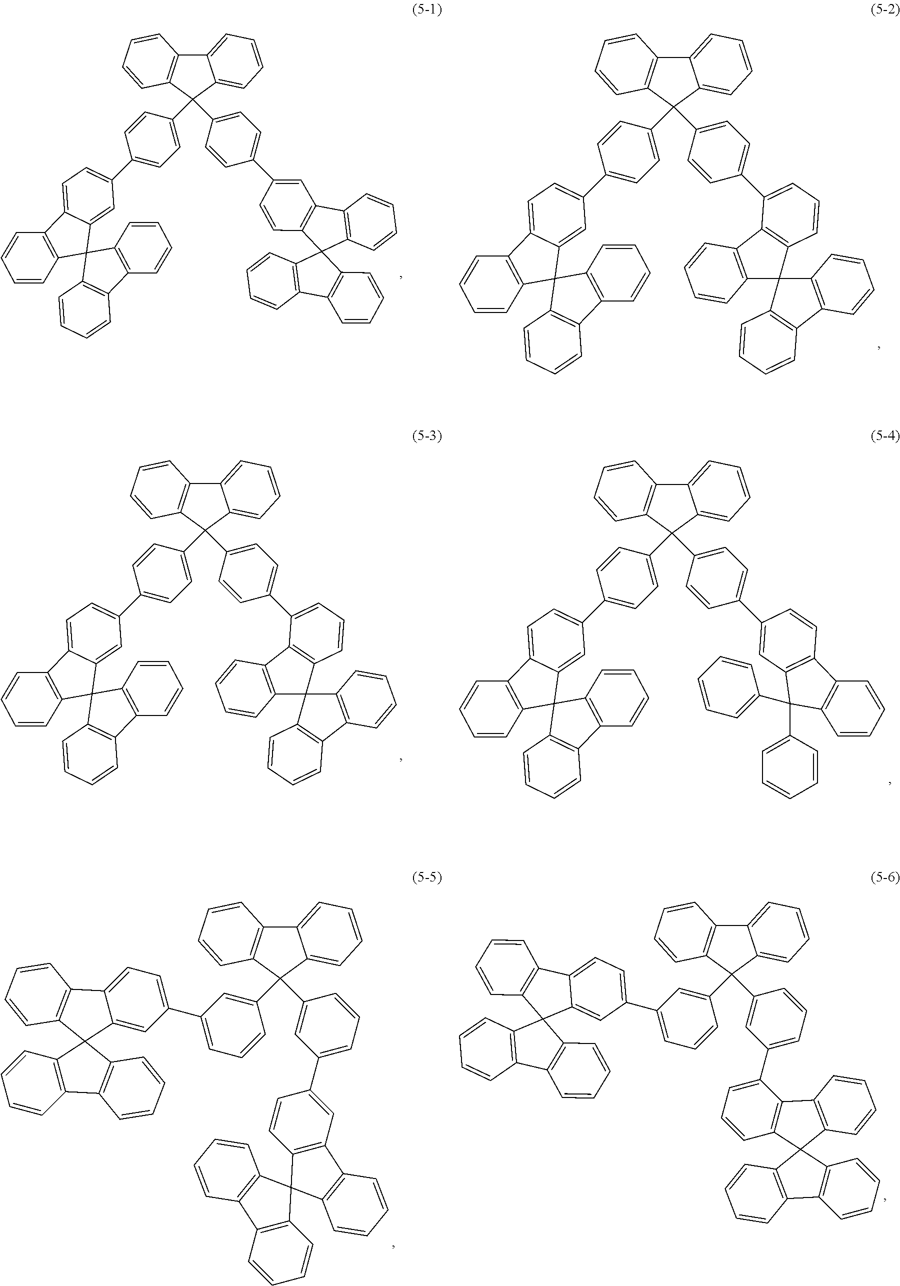

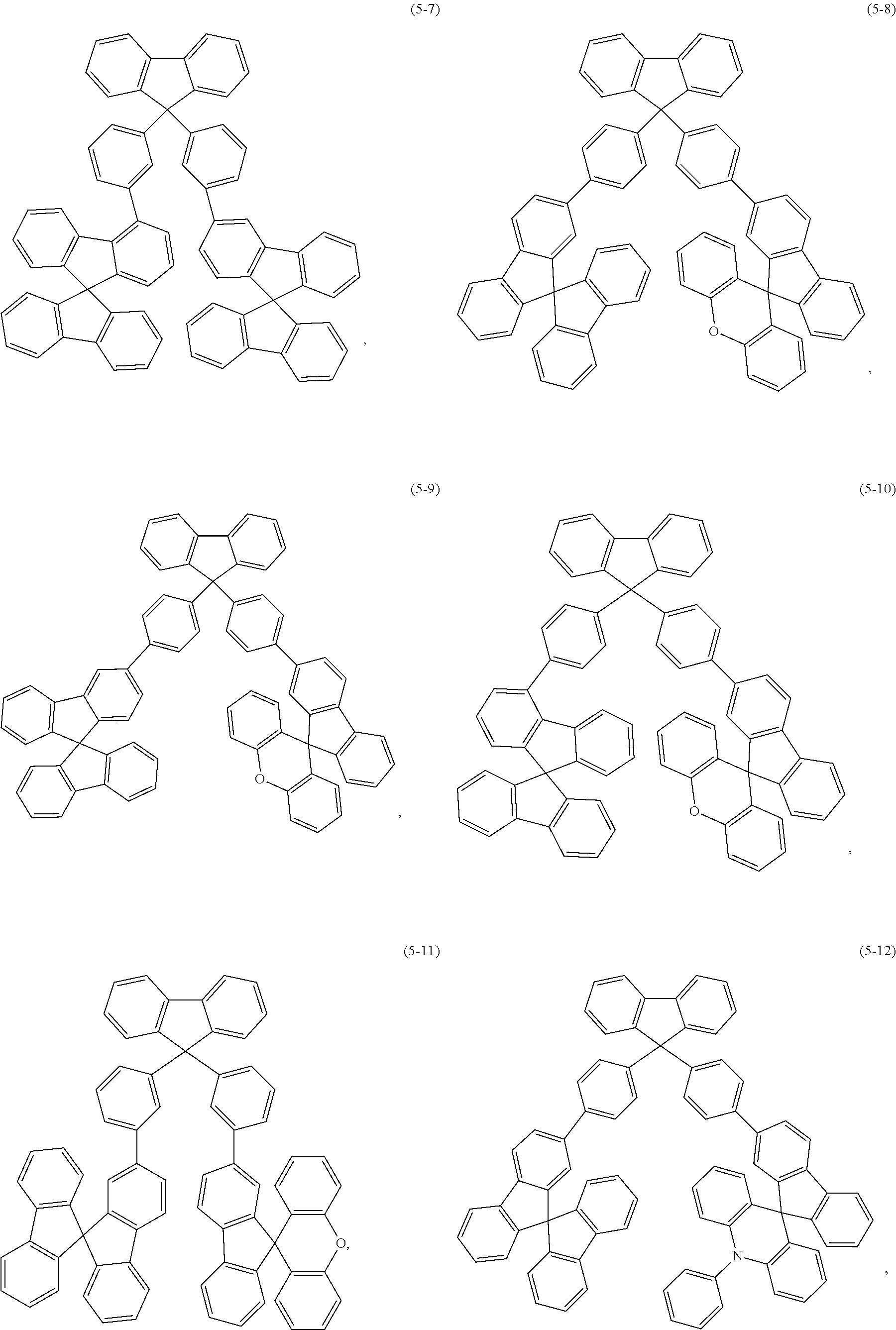

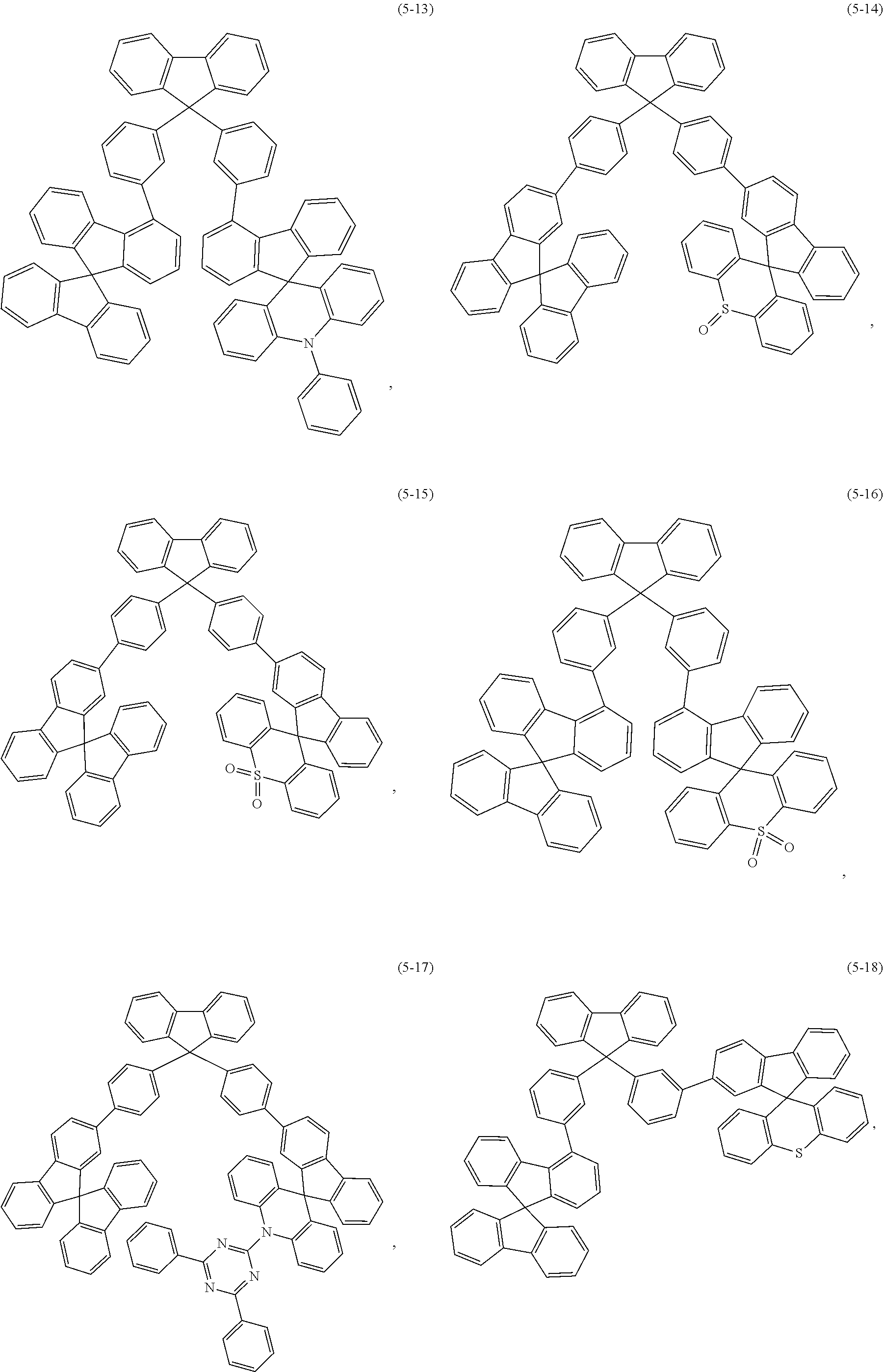

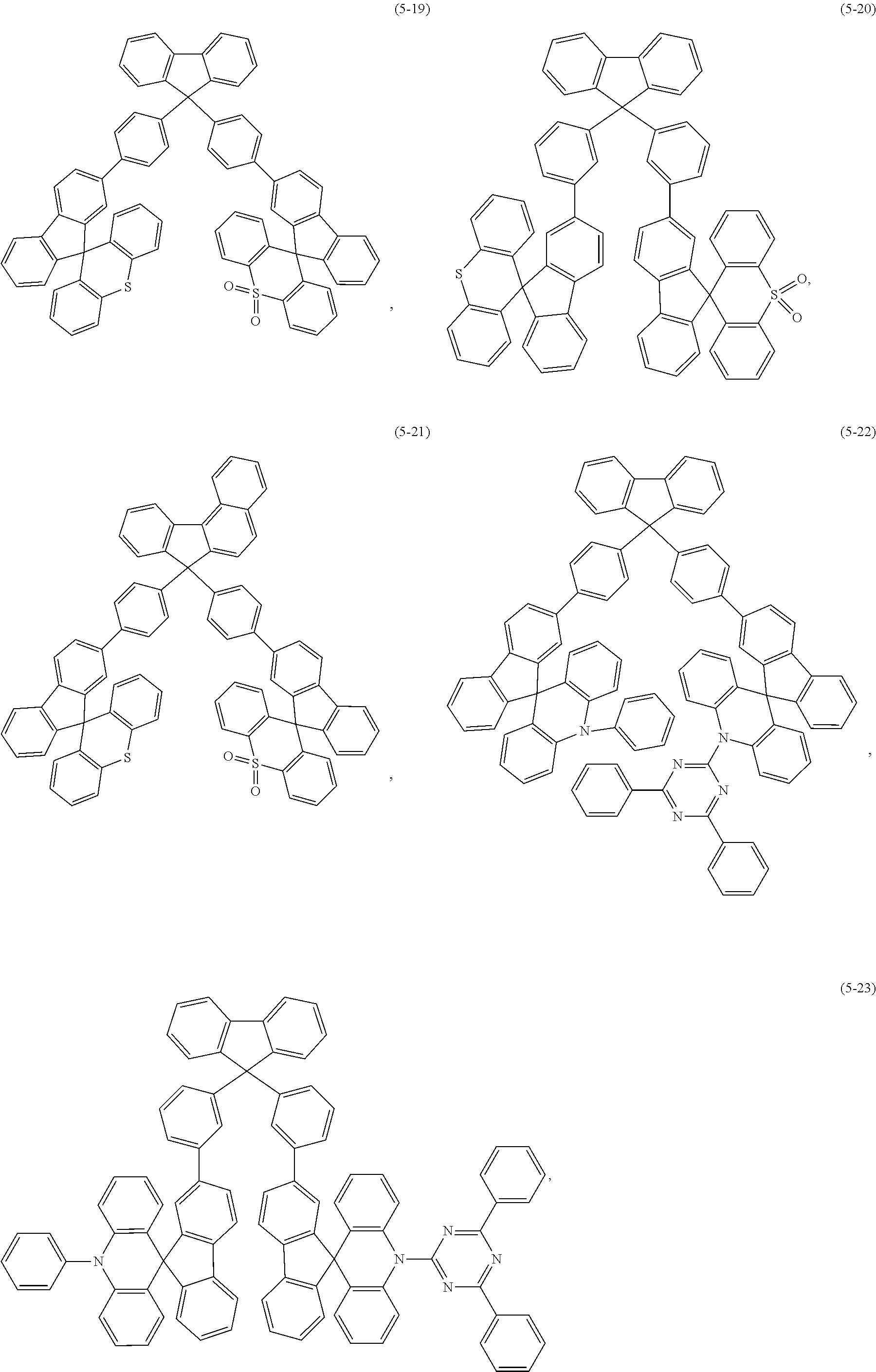

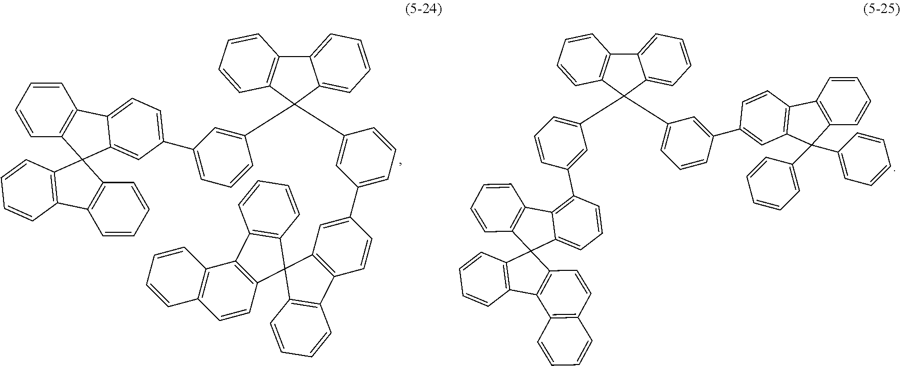

32. A formulation comprising at least one spirocyclic derivative according to claim 22 and at least one organic solvent.

33. The formulation according to claim 32, wherein the organic solvent is selected from aliphatic chain/ring substituted aromatic solvents, or aromatic ketone solvents, or aromatic ether solvents.

34. The formulation according to claim 32, wherein the organic solvent is selected from aliphatic chain/ring substituted aromatic solvents, or aromatic ketone solvents, or aromatic ether solvents.

35. The formulation according to claim 32, wherein the organic solvent is one selected from the group consisting of p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene, dibenzylether, and the like; solvents based on ketones: 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-demayone, 2,5-hexanedione, phorone, di-n-amyl ketone; aromatic ether solvents: 3-phenoxytoluene, butoxybenzene, benzylbutylbenzene, p-anisaldehyde dimethyl acetal, tetrahydro-2-phenoxy-2H-pyran, 1,2-dimethoxy 4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents: alkyl octoate, alkyl sebacate, alkyl stearate, alkyl benzoate, alkyl phenylacetate, alkyl cinnamate, alkyl oxalate, alkyl maleate, alkyl lactone and alkyl oleate.

36. The formulation according to claim 32, wherein the organic solvent is one selected from the group consisting of 2-nonanone, 3-nonanone, 5-nonanone, 2-demayone, 2,5-hexanedione, 2,6,8-trimethyl-4-demayone, phorone, di-n-pentyl ketone, and the like; or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethyl ether alcohol ethyl methyl ether, triethylene glycol butyl methyl ether, tripropylene glycol dimethyl ether, and tetraethylene glycol dimethyl ether.

37. An organic electronic device comprises the spirocyclic derivative according to claim 22.

38. The organic electronic device according to claim 37, wherein the organic electronic device is one selected from an organic light-emitting diode, an organic photovoltaic, an organic light emitting electrochemical cell, an organic field effect transistor, an organic light emitting field effector, an organic laser, an organic spin electron device, an organic sensor, and an organic plasmon emitting diode.

39. The organic electronic device according to claim 37 is an electroluminescence device comprising a substrate, an anode, a light-emitting layer, and a cathode, wherein the light-emitting layer comprises the spirocyclic derivative having the following general formula (I) 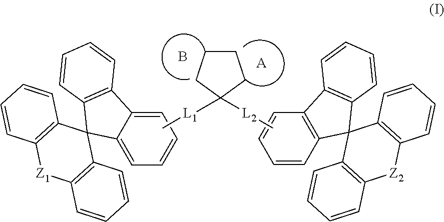
wherein, L₁ or L₂ is a single bond, an aromatic group containing 6 to 40 carbon atoms, or a heteroaromatic group containing 3 to 40 carbon atoms.
A or B is an aromatic group containing 6 to 20 carbon atoms or a heteroaromatic group containing 3 to 20 carbon atoms.
Z₁ or Z₂ is selected from a single bond, N(R), B(R), C(R)₂, Si(R)₂, O, S, C═N(R), C═C(R)₂, P(R), P(═O)R, S═O, or SO₂, or absent,
the hydrogen atoms on L₁, L₂, A, B and the spirocyclic derivative can be substituted by R;
R is an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms, and one or more positions of R may be substituted by H, D, F, CN, alkyl, aralkyl, alkenyl, alkynyl, nitrile group, amino, nitro, acyl, alkoxy, carbonyl, sulfonyl, cycloalkyl, or hydroxy.

40. The organic electronic device according to claim 39, wherein the light-emitting layer further comprises a light-emitting material selected from a fluorescent light emitter, a phosphorescent light emitter, a TADF material or a light-emitting quantum dot.

41. The organic electronic device according to claim 37, further comprising a hole-transport layer, wherein
the hole-transport layer comprises the spirocyclic derivative having the following general formula (I) 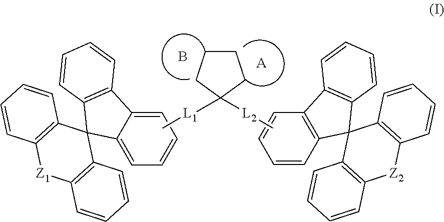
wherein, L₁ or L₂ is a single bond, an aromatic group containing 6 to 40 carbon atoms, or a heteroaromatic group containing 3 to 40 carbon atoms.
A or B is an aromatic group containing 6 to 20 carbon atoms or a heteroaromatic group containing 3 to 20 carbon atoms.
Z₁ or Z₂ is selected from a single bond, N(R), B(R), C(R)₂, Si(R)₂, O, S, C═N(R), C═C(R)₂, P(R), P(═O)R, S═O, or SO₂, or absent,
the hydrogen atoms on L₁, L₂, A, B and the spirocyclic derivative can be substituted by R;
R is an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 atoms, and one or more positions of R may be substituted by H, D, F, CN, alkyl, aralkyl, alkenyl, alkynyl, nitrile group, amino, nitro, acyl, alkoxy, carbonyl, sulfonyl, cycloalkyl, or hydroxy.
